(19) United States
(12) Patent Application Publication
(10) Pub. No.: US 2025/0186496 A1
(43) Pub. Date: Jun. 12, 2025

(54) METHODS AND COMPOSITIONS FOR CONTROLLING T CELL ACTIVATION

(71) Applicant: The General Hospital Corporation, Boston, MA (US)

(72) Inventors: Marcela V. Maus, Lexington, MA (US); Michael Charles Kann, Somerville, MA (US); Max Jan, Boston, MA (US)

(73) Assignee: The General Hospital Corporation, Boston, MA (US)

(21) Appl. No.: 18/844,805

(22) PCT Filed: Mar. 6, 2023
(86) PCT No.: PCT/US2023/063775

§ 371 Date: Sep. 6, 2024

Related U.S. Application Data
(60) Provisional application No. 63/317,292, filed on Mar. 7, 2022.

(51) Int. Cl. A61K 35/17; A61K 31/454; A61K 40/11; A61K 40/31; A61K 40/42; A61P 35/00; C07K 14/54; C12N 15/86

(52) U.S. Cl. CPC A61K 35/17; A61K 31/454; A61K 40/11; A61K 40/31; A61K 40/4211; A61K 40/4234; A61P 35/00; C07K 14/5418; C12N 15/86; A61K 2239/13; A61K 2239/17; A61K 2239/21; C07K 2319/81; C07K 2319/95; C12N 2740/15043; C12N 2750/14143; C12N 2830/002

(57) ABSTRACT

The disclosure is directed, in part, to novel drug-responsive T cell factors providing temporal and/or spatial control over T cell (e.g., CAR T cell) activation and/or proliferation, T cells comprising the T cell factors, nucleic acid encoding the T cell factors, and methods for using and producing the same.

### RELATED APPLICATION

This application claims the benefit under 35 U.S.C. § 119(e) to U.S. Provisional Application No. 63/317,292, filed Mar. 7, 2022, entitled “Method and Compositions for Controlling T Cell Activation”, the entire contents of which are incorporated herein by reference.

### FEDERALLY SPONSORED RESEARCH

This invention was made with Government support under R01CA252940, awarded by the National Institute of Health, and K08CA255932, awarded by the National Institute of Health. The Government has certain rights in the invention.

### BACKGROUND

In recent years, genetically modified CAR T-cells have made incredible clinical progress in the treatment of hematologic malignancies. Cytokines play an important role in CAR-T cell activation. For example, IL-7 is a non-redundant cytokine that is required in early T-cell development and T-cell homeostasis. It is produced by the non-hematopoietic stromal cells and mediates co-stimulatory proliferative and anti-apoptotic signaling through the activation of phosphoinositide 3-kinase (PI3K) and the Janus kinase (JAK)-signal transducer and activator of transcription (STAT) pathway; specifically, STAT5. IL-7 is important for the homeostatic expansion of naïve T-cells as well as the survival and proliferation of memory T-cells. However, constitutive expression or over-expression of IL-7 can lead to CAR-T toxicity.

### SUMMARY

The disclosure is directed, in part, to compositions and methods for controlling the activity and/or proliferation of a T cell using a drug-responsive T cell proliferation and/or homeostasis factor (T cell factor). Expression of a T cell factor (e.g., comprising a cytokine domain) in a CAR-T cell can, e.g., promote T cell activity or maintain T cell homeostasis in vivo (e.g. in a human subject). In some embodiments, a T cell factor is membrane-bound and comprises a cytokine domain. Without wishing to be bound by theory, a membrane-bound T cell factor comprising a cytokine domain may increase pro-proliferative, anti-apoptotic, and/or pro-T cell activation signaling to the T cell on which it is situated and/or to T cells to which the T cell factor binds. T cell factors of the disclosure provide spatio-temporal control over promotion of T cell activity and/or maintenance of T cell homeostasis. For example, in some embodiments T-cells (e.g., CAR T cells) of the disclosure are engineered to deliver a spatially and temporally controllable cytokine domain, e.g., an IL-7 protein or portion thereof, by using a membrane-bound T cell factor comprising a degron domain. In some embodiments, a T cell factor comprising a degron domain is degraded in the presence of a drug, e.g., a thalidomide analog. In some embodiments, the thalidomide analog is lenalidomide. In some embodiments, the degron comprises is a zinc finger degron. In some embodiments, the degron domain is a cereblon (CRBN) polypeptide substrate domain.

In some aspects, the present application discloses a drug-responsive T cell factor comprising: a cytokine domain; and a degron domain. In some embodiments, the cytokine domain comprises a cytokine or portion thereof that promotes proliferation of a T cell. In some embodiments, the cytokine domain comprises a cytokine or portion thereof that promotes homeostasis of a T cell. In some embodiments, the cytokine domain comprises a cytokine or portion thereof that acts as an anti-apoptotic signal for a T cell. In some embodiments, the cytokine domain comprises a cytokine or portion thereof that promotes homeostatic expansion of a T cell. In some embodiments, the cytokine domain comprises a cytokine or portion thereof that promotes Th1, Th2, or Th17 differentiation. In some embodiments, the cytokine domain comprises a cytokine or portion thereof that activates phosphoinositide 3-kinase (PI3K) signaling and/or Janus kinase (JAK)-signal transducer and activator of transcription (STAT) pathway signaling. In some embodiments, the cytokine domain comprises a cytokine or portion thereof that activates STAT5.

In some embodiments, the cytokine domain comprises an IL-2 family cytokine or a portion thereof. In some embodiments, the cytokine domain comprises IL-2, IL-4, IL-7, IL-9, IL-15, IL-21, or a portion of any thereof. In some embodiments, the cytokine domain comprises IL-7 or a portion thereof. In some embodiments, the cytokine or portion thereof is a human cytokine or a portion of a human cytokine. In some embodiments, the cytokine domain comprises an amino acid sequence of any one of SEQ ID NOs: 2-5, or a variant thereof. In some embodiments, the T cell factor further comprises a transmembrane or hinge domain. In some embodiments, the transmembrane or hinge domain is situated between the cytokine domain and the CRBN polypeptide substrate domain.

In some embodiments, the transmembrane or hinge domain comprises a CD80 transmembrane/hinge domain. In some embodiments, the transmembrane or hinge domain comprises an amino acid sequence of any one of SEQ ID NOs: 47-48, or a variant thereof.

In some embodiments, the degron domain comprises a cereblon (CRBN) polypeptide substrate domain capable of binding CRBN in response to the drug. In some embodiments, the CRBN polypeptide substrate domain binding to CRBN promotes degradation of the T cell factor.

In some embodiments, degradation is mediated by the ubiquitin-pathway. In some embodiments, the T cell factor further comprises the drug. In some embodiments, the drug is a small molecule drug. In some embodiments, the drug is an FDA-approved drug. In some embodiments, the drug can be administered to a human subject in a clinical setting. In some embodiments, the drug is a thalidomide analog. In some embodiments, the drug is an immunomodulatory imide drug (IMiD). In some embodiments, the drug is selected from the group consisting of thalidomide, lenalidomide and pomalidomide.

In some embodiments, the CRBN polypeptide substrate domain is selected from the group consisting of IKZF1, IKZF3, CK1α, ZFP91, GSPT1, MEIS2, GSS, E4F1, ZN276, ZN517, ZN582, ZN653, ZN654, ZN692, ZN787, ZN827, or a fragment thereof that is capable of drug-inducible binding a CRBN polypeptide. In some embodiments, the CRBN polypeptide domain comprises IKZF3 or a fragment thereof that is capable of drug-inducible binding a CRBN polypeptide. In some embodiments, the CRBN polypeptide domain comprises amino acids 130-145, amino acids 169-189, or amino acids 130-189 of IKZF3 (SEQ ID NO: 21). In some embodiments, the CRBN polypeptide substrate domain comprises a hybrid fusion polypeptide comprised of ten or more residues of a non-IKZF3 C2H2 zinc finger degron sequence flanked by an N-terminal IKZF3 degron sequence and a C-terminal IKZF3 degron sequence. In some embodiments, the N-terminal IKZF3 degron sequence comprises or is amino acids 130-145 (SEQ ID NO: 23) of IKZF3 and/or wherein the C-terminal IKZF3 degron sequence comprises or is amino acids 169-189 (SEQ ID NO: 25) of IKZF3. In some embodiments, the non-IKZF3 C2H2 zinc finger degron sequence is a ZFP91 sequence. In some embodiments, the CRBN polypeptide substrate domain is SEQ ID NO: 22. In some embodiments, the CRBN polypeptide substrate domain is SEQ ID NO: 27. In some embodiments, the T cell factor comprises an amino acid sequence of SEQ ID NO: 29 or a variant thereof.

In some aspects, the present application discloses a nucleic acid molecule comprising a sequence encoding the T cell factor described herein. In some embodiments, the nucleic acid molecule comprises a first polynucleotide encoding the T cell factor and a second polynucleotide encoding a chimeric antigen receptor (CAR). In some embodiments, the CAR comprises an extracellular antigen-binding domain, a transmembrane domain (TMD), a co-stimulatory domain, and a signaling domain. In some embodiments, the nucleic acid molecule is a vector. In some embodiments, the vector is a plasmid or a viral vector. In some embodiments, the viral vector is a lentiviral or adeno-associated viral vector. In some aspects, the present application discloses a mammalian cell comprising the T cell factor described herein or the nucleic acid molecule described herein.

In some embodiments, the mammalian cell overexpresses a CRBN polypeptide. In some embodiments, the overexpressed CRBN polypeptide is targeted to the plasma membrane with a targeting sequence derived from LAT, PAG, LCK, FYN, LAX, CD2, CD3, CD4, CDS, CD7, CD8a, PDl, SRC, or LYN. In some embodiments, the local concentration of the ubiquitin ligase CRL4^{CRBN} is increased at the plasma membrane, as compared to an appropriate control. In some embodiments, the mammalian cell is a T cell. In some embodiments, the cell is selected from the group consisting of a B cell, plasma cell, NK cell, NKT cell, innate lymphoid cell, macrophage, dendritic cell, monocyte, neutrophil, basophil, eosinophil, mast cell, hematopoietic progenitor cell, hematopoietic stem cell, other adult stem cell such as neural, cornea, muscle, skin, small intestine, colon, bone, mesenchyme, embryonic stem cell and an induced pluripotent stem cell.

In some embodiments, the cell is a chimeric antigen receptor (CAR) T cell. In some embodiments, the cell comprises a CAR comprising: an extracellular antigen-binding domain, a transmembrane domain (TMD), a co-stimulatory domain, and a signaling domain. In some embodiments, the cell comprises a first polynucleotide encoding the T cell factor and a second polynucleotide encoding the CAR. In some embodiments, the first polynucleotide and second polynucleotide are on the same nucleic acid molecule. In some embodiments, the first polynucleotide and second polynucleotide are on different nucleic acid molecules. In some embodiments, the CAR and the T cell factor are produced in the form of a single polypeptide, which is cleaved to generate separate CAR and therapeutic agent molecules. In some embodiments, the single polypeptide comprises a cleavable moiety between the CAR and the T cell factor. In some embodiments, the cleavable moiety comprises: a 2A peptide, a 2A ribosomal skip sequence, or an IRES. In some embodiments, the 2A peptide comprises P2A or T2A. In some embodiments, the CAR and the T cell factor are each constitutively expressed. In some embodiments, expression of the CAR and the T cell factor is driven by an elongation factor-1 alpha (EF1α) promoter. In some embodiments, the T cell factor is expressed under the control of an inducible promoter, which is optionally inducible by T cell receptor or CAR signaling. In some embodiments, the inducible promoter comprises the NFAT promoter. In some embodiments, the CAR is expressed under the control of a constitutive promoter and the T cell factor is expressed under the control of an inducible promoter, which is optionally inducible by T cell receptor or CAR signaling.

In some embodiments, the extracellular antigen-binding domain of the CAR comprises an antibody, a single chain antibody, a single domain antibody, or a ligand. In some embodiments, the extracellular antigen-binding domain binds to a tumor-associated antigen. In some embodiments, the tumor-associated antigen is selected from the group consisting of EGFR, EGFRvIII, CD19, CD37, BCMA, CEA, immature laminin receptor, TAG-72, HPV E6 and E7, BING-4, calcium-activated chloride channel 2, cyclin B1, 9D7, Ep-CAM, EphA3, her2/neu, telomerase, mesothelin, SAP-1, Survivin, BAGE family, CAGE family, GAGE family, MAGE family, SAGE family, XAGE family, NY-ESO-1/LAGE-1, PRAME, SSX-2, Melan-A/MART-1, gp100/pmel17, tyrosinase, TRP-1/-2, MC1R, BRCA1/2, CDK4, MART-2, p53, Ras, MUC1, TGF-βRII, IL-15, IL13Ra2, or CSF1R.

In some embodiments, wherein the transmembrane domain of the CAR comprises a CD8 hinge/transmembrane domain, which optionally comprises the sequence of SEQ ID NO: 47, or a variant thereof.

In some embodiments, the signaling domain comprises a CD3ζ domain, a CD3 gamma domain, a CD3 delta domain, a CD3 epsilon domain, a FcR gamma domain, a FcR beta domain, a CDS domain, a CD79a domain, a CD79b domain, a CD66d domain, a CD4 domain, a CD8 domain, a Dap10 domain, and a Dap-12 domain, wherein the signaling domain optionally comprises the sequence of SEQ ID NO: 50, or a variant thereof.

In some embodiments, the co-stimulatory domain comprises a CD28 co-stimulatory domain or a 4-1BB co-stimulatory domain, wherein the co-stimulatory domain optionally comprises the sequence of SEQ ID NO: 49 or a variant thereof.

In some aspects, the present disclosure relates to a pharmaceutical composition comprising the mammalian cell described herein or nucleic acid described herein.

In some aspects the present disclosure relates to a method for treating a subject with a chimeric antigen receptor (CAR) cellular therapy, the method comprising administering to the subject a drug-responsive CAR T cell as described herein. In some embodiments, the method further comprises administering the drug. In some embodiments, the method further comprises identifying a CAR cellular therapy side effect in the subject. In some embodiments, the method further comprises administering the drug after the CAR cellular therapy side effect is identified in the subject. In some embodiments, the drug is a small molecule drug. In some embodiments, the drug is an FDA-approved drug. In some embodiments, the drug can be administered to a human subject in a clinical setting. In some embodiments, the drug is a thalidomide analog. In some embodiments, the drug is an immunomodulatory imide drug (IMiD). In some embodiments, the drug is selected from the group consisting of thalidomide, lenalidomide and pomalidomide. In some embodiments, the subject has or is at risk of developing cancer. In some embodiments, the cancer is glioblastoma, glioma, leukemia, lymphoma, multiple myeloma, or a solid tumor. In some embodiments, the leukemia is acute myeloid leukemia (AML), chronic myeloid leukemia (CML), acute lymphocytic leukemia (ALL), or chronic lymphocytic leukemia (CLL); wherein the lymphoma is diffuse large B-cell lymphoma (DLBCL), follicular lymphoma, chronic lymphocytic leukemia (CLL), small lymphocytic lymphoma (SLL), mantle cell lymphoma (MCL), marginal zone lymphomas, Burkitt lymphoma, hairy cell leukemia (HCL), and T cell lymphoma (e.g., peripheral T cell lymphoma (PTCL), including cutaneous T cell lymphoma (CTCL) and anaplastic large cell lymphoma (ALCL)); or wherein the solid tumor is adrenocortical tumor, alveolar soft part sarcoma, carcinoma, chondrosarcoma, colorectal carcinoma, desmoid tumors, desmoplastic small round cell tumor, endocrine tumors, endodermal sinus tumor, epithelioid hemangioendothelioma, Ewing sarcoma, germ cell tumors (solid tumor), giant cell tumor of bone and soft tissue, hepatoblastoma, hepatocellular carcinoma, melanoma, nephroma, neuroblastoma, non-rhabdomyosarcoma soft tissue sarcoma (NRSTS), osteosarcoma, paraspinal sarcoma, renal cell carcinoma, retinoblastoma, rhabdomyosarcoma, synovial sarcoma, or Wilms tumor.

In some aspects, the present application discloses a method of producing a drug-responsive chimeric antigen receptor (CAR) T cell, comprising: contacting a CAR T cell with a nucleic acid molecule described herein, thereby producing a drug-responsive CAR T cell. In some embodiments, contacting comprises transfection, transduction, and/or electroporation of the nucleic acid molecule. In some embodiments, the nucleic acid molecule is integrated into the genome of the CAR T cell. In some embodiments, the T cell factor encoded by the nucleic acid molecule is expressed in the drug-responsive CAR T cell. In some embodiments, the method, further comprises providing the CAR T cell. In some embodiments, providing the CAR T cell comprises contacting a T cell with a nucleic acid molecule encoding the CAR.

In some embodiments, the nucleic acid molecule encoding the CAR is a vector. In some embodiments, the vector is a plasmid or a viral vector. In some embodiments, the viral vector is a lentiviral or adeno-associated viral vector. In some embodiments, wherein the CAR is encoded upon the same nucleic acid molecule that encodes the T cell factor. In some embodiments, the nucleic acid molecule encoding the CAR and the nucleic acid molecule encoding the T cell factor are different nucleic acid molecules.

In some embodiments, the method further comprises selecting for a drug-responsive CAR T cell. In some embodiments, selecting comprises using cell sorting, e.g., Fluorescence Activated Cell Sorting (FACS). In some embodiments, selecting selects for cells comprising a nucleic acid molecule encoding the T cell factor. In some embodiments, selecting selects for cells comprising a nucleic acid molecule encoding the CAR. In some embodiments, the method further comprises contacting a T cell with a nucleic acid molecule comprising a sequence encoding a CRBN polypeptide. In some embodiments, the sequence encoding a CRBN polypeptide is operably linked to a promoter configured to overexpress the CRBN polypeptide in the T cell. In some embodiments, the CRBN polypeptide encoded by the nucleic acid molecule is targeted to the plasma membrane with a targeting sequence derived from LAT, PAG, LCK, FYN, LAX, CD2, CD3, CD4, CDS, CD7, CD8a, PDl, SRC, or LYN. In some embodiments, the local concentration of the ubiquitin ligase CRL4^{CRBN} is increased at the plasma membrane, as compared to an appropriate control. In some embodiments, the nucleic acid molecule encoding the CRBN polypeptide is a vector. In some embodiments, the vector is a plasmid or a viral vector. In some embodiments, the viral vector is a lentiviral or adeno-associated viral vector. In some embodiments, the CRBN polypeptide is encoded upon the same nucleic acid molecule that encodes the T cell factor. In some embodiments, the CRBN polypeptide is encoded upon the same nucleic acid molecule that encodes the CAR. In some embodiments, the CRBN polypeptide is encoded upon the same nucleic acid molecule that encodes the T cell factor and the CAR. In some embodiments, the nucleic acid molecule encoding the CRBN polypeptide, the nucleic acid encoding the CAR, and the nucleic acid molecule encoding the T cell factor are different nucleic acid molecules. In some embodiments, selecting selects for cells comprising a nucleic acid molecule encoding the CRBN polypeptide.

### BRIEF DESCRIPTION OF THE DRAWINGS

FIGS. 1A-1B show cartoons illustrating an exemplary CAR and T cell factor construct and the CAR and the T cell factor inserted into the membrane. FIG. 1A shows a nucleic acid construct encoding a CD19 CAR with and without an exemplary T cell factor comprising an IL-7 cytokine domain. The 2A elements are self-cleaving peptides that allow the CAR and the T cell factor to physically separate after translation. FIG. 1B show a cartoon of the CAR and the T cell factor inserted into the membrane of a cell (e.g., a T-cell).

FIGS. 2A-2B show graphs of surface expression of exemplary T cell factor mbdIL-7 on primary human T cells (FIG. 2A) and STAT5 signaling (FIG. 2B) over increasing concentrations of an exemplary thalidomide analog, lenalidomide.

FIG. 3 shows a graph of population doublings over time for T cells expressing a CD19 CAR in the presence of exogenous IL-2 and T cells expressing a CD19 CAR and exemplary T cell factor mdbIL-7 in the absence of exogenous IL-2.

FIGS. 4A-4B show graphs of T cell phenotype in T cells expressing a CAR and exemplary T cell factor mbdlIL-7. FIG. 4A shows a graph of expression of markers (CCR7 and CD45RA) on T cells that are indicative of differentiation state (e.g., central memory (CM), Naïve, Effector memory (EffMem), and Effector (Eff)). FIG. 4B shows a graph of the relative proportions of each differentiation state in a population of a CAR19 (CAR-T cells with CD19) expressing T cells without exemplary T cell factor mdbIL7 (top) or with exemplary T cell factor mbdIL7 (bottom).

FIGS. 5A-5B show graphs of percent lysis of CD19-expressing cells in vitro by CAR19 cells with and without exemplary T cell factor mdbIL7. FIG. 5A shows percent lysis over varying ratios of CAR19 cells to CD19-expressing NALM6 cells, and FIG. 5B shows percent lysis over time at a 1:1 ratio of NALM6:CAR19 cells.

FIG. 6 shows mbdIL7 enhances long term proliferation and expansion while maintaining OFF-switch control. CAR19 or CAR19mbdIL7 were exposed weekly at a 1:1 ratio to irradiated CD19+K562s in the presence or absence of 1 μM lenalidomide and expansion was tracked via cell counting and flow cytometry.

FIG. 7 shows graphs of cell type score characterizing T cell phenotype of CAR19 cells with and without exemplary T cell factor mdbIL7.

FIGS. 8A-8B show a graph and heatmap illustrating the relative change in expression of genes between CAR19 cells with and without exemplary T cell factor mbdIL7.

FIGS. 9A-9C show graphs of exemplary cancer CD19 positive cell line (JeKO-1) abundance in mice over time (FIG. 9B) and CD3+ cell abundance over time (FIG. 9C) after administration of CAR19 cells with and without exemplary T cell factor mdbIL7. FIG. 9A shows a schematic of treatment of the mice.

FIG. 10 shows mbdIL7 is quickly and effectively degraded in a reversible fashion. CAR19 mbdIL7 cells were exposed to different doses of lenalidomide (or 1 μM for time course assays) and surface abundance of mbdIL7 was measured via flow cytometry.

FIGS. 11A-11B show mbdIL7 allows for the exclusion of IL-2 in CAR production, resulting in a superior product phenotype. FIG. 11A shows CAR19 mbdIL7 expand at the same rate as control CAR without IL-2. FIG. 12B shows CAR19 mbdIL7 are enriched in Naïve and Central Memory phenotype T cells.

FIG. 12 shows CAR19 mbdIL7 have higher activation potential than CAR19. CAR19 and CAR19 mbdIL7 were co cultured with CD19+/− Nalm6 and K562 and CD69 expression was measured by flow cytometry.

FIGS. 13A-13C show mbdIL7 is cleaved from the T cell surface in a state-dependent manner. FIG. 13A shows cytokine expression in CAR T cells expressing either control CAR19 or CAR19 mbdIL-7 cocultured with CD19+ Nalm6. Similar cytokine profiles were observed between the two CARs, however, sIL-7 was detected in supernatant of the mbdIL7 CAR. FIG. 13B shows the cleavage of IL-7 was effectively controlled by lenalidomide in a dose-dependent manner. FIG. 13C show no appreciable effects of lenalidomide in CAR19 mbdIL7 that were not experiencing antigen exposure.

FIGS. 14A-14C show in vivo control of mdIL7 CAR19 proliferation by pomalidomide (Pom) administration. FIG. 14A shows a schematic of mice being injected with Jurkat cells, which were transduced with a construct encoding for a luciferase-tagged version of mbdIL7 (mbdIL7-Luc) and allowed to engraft for 25 days.

FIG. 14B shows reversible decrease in concentration of mdIL7 CAR19 after administration of pomalidomide. FIG. 14C shows luciferase detection in mice in response to Pom.

### DETAILED DESCRIPTION

The disclosure provides, in part, T cell factors that alter T cell (e.g., CAR T cell) differentiation and/or phenotype, activate T cells, increase T cell persistence, and/or increase T cell expansion in a manner that can be controlled (e.g., spatial and/or temporal control, e.g., in response to a subject's condition) by administration of a drug. The disclosure further provides, in part, cells comprising said T cell factors (e.g., drug-responsive CAR T cells), nucleic acids encoding said T cell factors, methods for producing said cells, and methods for using any thereof (e.g., methods of treating a subject by administering any thereof).

The disclosure is directed, in part, to the recognition that some patients fail to have sustained responses to CAR T cell therapy in part to due limited persistence of the administered CAR T cells. Cytokine treatment (e.g., IL-2 and/or IL-15), e.g., in combination with CAR T therapy, may be used to increase CAR T cell survival and/or increase persistence, but this combination of T cell activation and high levels of systemic cytokines can lead to toxicities. The disclosure is directed, in part, to the discovery that CAR T cell persistence can be enhanced while mitigating potential cytokine toxicities by controlling T cell activation using a drug-responsive T cell factor. In some embodiments, a drug-responsive T cell factor comprises a cytokine domain and a degron domain which, in the presence of a drug, results in the degradation of the T cell factor and an end to the signaling effect of the cytokine domain. In some embodiments, the cytokine domain of a T cell factor comprises IL-7 or a functional portion thereof. In some embodiments, the degron domain of a T cell factor comprises a cereblon (CRBN) polypeptide substrate domain.

### General Definitions

The terms “decrease,” “reduced,” “reduction,” or “inhibit” are all used herein to mean a decrease by a statistically significant amount. In some embodiments, “reduce,” “reduction,” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. Where applicable, a decrease can be preferably down to a level accepted as within the range of normal for an individual without a given disorder. The terms “increased,” “increase,” “enhance,” or “activate” are all used herein to mean an increase by a statically significant amount. In some embodiments, the terms “increased,” “increase,” “enhance,” or “activate” can mean an increase of at least 10% as compared to a reference level, for example, an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

A “disease” is a state of health of an animal, for example, a human, wherein the animal cannot maintain homeostasis, and wherein if the disease is not ameliorated, then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “tumor antigen”, “tumor-associated antigen” and “cancer antigen” are used interchangeably to refer to antigens that are differentially expressed by cancer cells and can thereby be exploited in order to target cancer cells. Cancer antigens are antigens that can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), and fusion proteins resulting from internal deletions or chromosomal translocations. Still other cancer antigens can be encoded by viral genes such as those carried on RNA and DNA tumor viruses. Many tumor antigens have been defined in terms of multiple solid tumors: MAGE 1, 2, & 3, defined by immunity; MART-1/Melan-A, gp100, carcinoembryonic antigen (CEA), human epidermal growth factor receptor (HER2), mucins (i.e., MUC-1), prostate-specific antigen (PSA), and prostatic acid phosphatase (PAP). In addition, viral proteins such as some encoded by hepatitis B (HBV), Epstein-Barr (EBV), and human papilloma (HPV) have been shown to be important in the development of hepatocellular carcinoma, lymphoma, and cervical cancer, respectively. In some embodiments, the tumor-associated antigen is selected from CD19, CD37, CEA, CD79, gp100, EGFR, FR-α, HER2, EpHA2, Glypican-3, EGFR806, PSCA, IL-13Ra2, MUC1, MUC16, MAGE-A1/3/4, LMP1, CD171, Claudin 18.2, DR5, EpCAM, DLL-3, GD2, c-MET, VEGFR2, AFP, Nectin4/FAP, EGFRvIII, CD20, Lewis Y, CD22, BCMA, PSMA, AXL, CD80/86, GPRC5D, or mesothelin.

As used herein, the term “chimeric” refers to the product of the fusion of portions of at least two or more different polynucleotide molecules. In some embodiments, the term “chimeric” refers to a gene expression element produced through the manipulation of known elements or other polynucleotide molecules.

In some embodiments, “activation” can refer to the state of a T cell that has been sufficiently stimulated to induce detectable cellular proliferation. In some embodiments, activation can refer to induced cytokine production. In other embodiments, activation can refer to detectable effector functions.

At a minimum, an “activated T cell” as used herein is a proliferative T cell.

As used herein, the terms “specific binding” and “specifically binds” refer to a physical interaction between two molecules, compounds, cells and/or particles wherein the first entity binds to the second, target, entity with greater specificity and affinity than it binds to a third entity which is a non-target. In some embodiments, specific binding can refer to an affinity of the first entity for the second target, entity, which is at least 10 times, at least 50 times, at least 100 times, at least 500 times, at least 1000 times or more greater than the affinity for the third non-target entity under the same conditions. A reagent specific for a given target is one that exhibits specific binding for that target under the conditions of the assay being utilized. A non-limiting example includes an antibody, or a ligand, which recognizes and binds with a cognate binding partner (for example, a stimulatory and/or costimulatory molecule present on a T cell) protein.

A “stimulatory ligand,” as used herein, refers to a ligand that when present on an antigen presenting cell (APC) (e.g., a macrophage, a dendritic cell, a B-cell, an artificial APC, and the like) can specifically bind with a cognate binding partner (referred to herein as a “stimulatory molecule” or “costimulatory molecule”) on a T cell, thereby mediating a primary response by the T cell, including, but not limited to, proliferation, activation, initiation of an immune response, and the like. Stimulatory ligands are well-known in the art and encompass, inter alia, an MHC Class I molecule loaded with a peptide, an antiCD3 antibody, a superagonist anti-CD28 antibody, and a superagonist anti-CD2 antibody.

A “stimulatory molecule,” as the term is used herein, means a molecule on a T cell that specifically binds with a cognate stimulatory ligand present on an antigen presenting cell. “Co-stimulatory ligand,” as the term is used herein, includes a molecule on an APC that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, 4-1BBL, OX40L, CD7, B7-1 (CD80), B7-2 (CD86), PD-L1, PD-L2, inducible co-stimulatory ligand (ICOS-L), intercellular adhesion molecule (ICAM), CD30L, CD40, CD70, CD83, HLA-G, MICA, MICB, HVEM, lymphotoxin beta receptor, 3/TR6, IL T3, IL T4, HVEM, an agonist or antibody that binds Toll-like receptor and a ligand that specifically binds with B7-H3. A co-stimulatory ligand also can include, but is not limited to, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include but are not limited to an MHC class I molecule, BTLA, a Toll-like receptor, CD27, CD28, 4-1BB, OX40, CD30, CD40, PD-1, ICOS, lymphocyte function associated antigen-1 (LFA-1), CD2, CD7, LIGHT, NKG2C, B7-H3, and CD83.

In some embodiments, the term “engineered” and its grammatical equivalents as used herein can refer to one or more human-designed alterations of a nucleic acid, e.g., the nucleic acid within an organism's genome. In another embodiment, engineered can refer to alterations, additions, and/or deletion of genes. An “engineered cell” can refer to a cell with an added, deleted and/or altered gene.

The term “cell” or “engineered cell” and their grammatical equivalents as used herein can refer to a cell of human or non-human animal origin.

As used herein, the term “operably linked” refers to a first polynucleotide molecule, such as a promoter, connected with a second transcribable polynucleotide molecule, such as a gene of interest, where the polynucleotide molecules are so arranged that the first polynucleotide molecule affects the function of the second polynucleotide molecule. The two polynucleotide molecules may or may not be part of a single contiguous polynucleotide molecule and may or may not be adjacent. For example, a promoter is operably linked to a gene of interest if the promoter regulates or mediates transcription of the gene of interest in a cell.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of ordinary skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as lie, Val, Leu, or Ala for one another), or substitution of one polar residue for another (such as between Lys and Arg; Glu and Asp; or Gin and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g., ligand-mediated receptor activity and specificity of a native or reference polypeptide is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; ile into Leu or into Val; Leu into ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, a polypeptide described herein (or a nucleic acid encoding such a polypeptide) can be a functional fragment of one of the amino acid sequences described herein. As used herein, a “functional fragment” is a fragment or segment of a peptide that retains at least 50% of the wildtype reference polypeptide's activity according to an assay known in the art or described below herein. A functional fragment can comprise conservative substitutions of the sequences disclosed herein.

In some embodiments, a polypeptide described herein can be a variant of a polypeptide or molecule as described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions, or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity of the non-variant polypeptide. A wide variety of PCR-based site-specific mutagenesis approaches are known in the art and can be applied by the ordinarily skilled artisan.

A variant amino acid or DNA sequence can be at least 80%, at least 85%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g., BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide directed site-specific mutagenesis procedures can be employed to provide an altered nucleotide sequence having particular codons altered according to the substitution, deletion, or insertion required. Techniques for making such alterations are well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of a polypeptide also can be substituted, generally with serine, to improve the oxidative stability of the molecule and prevent aberrant crosslinking. Conversely, cysteine bond(s) can be added to a polypeptide to improve its stability or facilitate oligomerization.

The term “polynucleotide” is used herein interchangeably with “nucleic acid molecule” to indicate a polymer of nucleosides. Typically a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically characterizes a specific nucleic acid. In some embodiments, the nucleic acid molecule is a heterologous nucleic acid molecule. As used herein the term, “heterologous nucleic acid molecule” refers to a nucleic acid molecule that does not naturally exist within a given cell.

A polynucleotide sequence presented herein is presented in a 5′ to 3′ direction unless otherwise indicated.

The term “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a non-polypeptide moiety covalently or noncovalently associated therewith is still considered a “polypeptide.” Exemplary modifications include glycosylation and palmitoylation. Polypeptides can be purified from natural sources, produced using recombinant DNA technology or synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

In some embodiments, a nucleic acid encoding a polypeptide as described herein (e.g., a CAR polypeptide) is comprised by a vector. In some of the aspects described herein, a nucleic acid sequence encoding a given polypeptide as described herein, or any module thereof, is operably linked to a vector. The term “vector,” as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example, in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene.

The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′ UTR) or “leader” sequences and 3′ UTR or “trailer” sequences, as well as intervening sequences (intrans) between individual coding segments (exons).

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

By “recombinant vector” is meant a vector that includes a heterologous nucleic acid sequence or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra-chromosomal DNA thereby eliminating potential effects of chromosomal integration.

As used herein, a “signal peptide” or “signal sequence” refers to a peptide at the N-terminus of a newly synthesized protein that serves to direct a nascent protein into the endoplasmic reticulum. In some embodiments, the signal peptide is a CDSCD8 or lgK signal peptide.

As used herein, the terms “treat,” “treatment,” “treating,” or “amelioration” refer to therapeutic treatments, wherein the object is to reverse, alleviate, ameliorate, inhibit, slow down, or stop the progression or severity of a condition associated with a disease or disorder, e.g., a non-solid tumor like mantle cell lymphoma. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side effects of the disease (including palliative treatment).

As used herein, the term “administering,” refers to the placement of a therapeutic or pharmaceutical composition as disclosed herein into a subject by a method or route that results in at least partial delivery of the agent at a desired site. Pharmaceutical compositions comprising agents as disclosed herein can be administered by any appropriate route that results in an effective treatment in the subject.

In some embodiments, the methods described herein relate administering to a subject having or diagnosed as having cancer, a plasma cell disease or disorder, or an autoimmune disease or disorder with a mammalian cell including any of the CAR polypeptides (and optional antibody reagents or cytokines) described herein, or a nucleic acid encoding any of the CAR polypeptides (and optional antibody reagents or cytokines) described herein. The CART cells described herein include mammalian cells including any of the CAR polypeptides (and optional antibody reagents or cytokines) described herein, or a nucleic acid encoding any of the CAR polypeptides (and optional antibody reagents or cytokines) described herein. As used herein, a “condition” refers to a cancer, a plasma cell disease or disorder, or an autoimmune disease or disorder. Subjects having a condition can be identified by a physician using current methods of diagnosing the condition. Symptoms and/or complications of the condition, which characterize these conditions and aid in diagnosis are well known in the art and include but are not limited to, fatigue, persistent infections, and persistent bleeding. Tests that may aid in a diagnosis of, e.g., the condition, but are not limited to, blood screening and bone marrow testing, and are known in the art for a given condition. A family history for a condition, or exposure to risk factors for a condition can also aid in determining if a subject is likely to have the condition or in making a diagnosis of the condition.

The compositions described herein can be administered to a subject having or diagnosed as having a condition. In some embodiments, the methods described herein include administering an effective amount of activated CAR T cells described herein to a subject in order to alleviate a symptom of the condition. As used herein, “alleviating a symptom of the condition” is ameliorating any condition or symptom associated with the condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. In some embodiments, the compositions described herein are administered systemically or locally. In a preferred embodiment, the compositions described herein are administered intravenously. In another embodiment, the compositions described herein are administered at the site of a tumor.

The term “effective amount” as used herein refers to the amount of activated CAR T cells needed to alleviate at least one or more symptom of the disease or disorder, and relates to a sufficient amount of the cell preparation or composition to provide the desired effect. The term “therapeutically effective amount” therefore refers to an amount of activated CART cells that is sufficient to provide a particular anti-condition effect when administered to a typical subject. An effective amount as used herein, in various contexts, would also include an amount sufficient to delay the development of a symptom of the disease, alter the course of a symptom disease (for example but not limited to, slowing the progression of a condition), or reverse a symptom of the condition. Thus, it is not generally practicable to specify an exact “effective amount.” However, for any given case, an appropriate “effective amount” can be determined by one of ordinary skill in the art using only routine experimentation.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of activated CART cells, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for bone marrow testing, among others. The dosage can be determined by a physician and adjusted, as necessary, to suit observed effects of the treatment.

Modes of administration can include, for example intravenous (iv) injection or infusion. The compositions described herein can be administered to a patient transarterially, intratumorally, intranodally, intraperitoneally or intramedullary. the In some embodiments, the compositions of T cells may be injected directly into a tumor, lymph node, or site of infection. In some embodiments, the compositions described herein are administered into a body cavity or body fluid (e.g., ascites, pleural fluid, peritoneal fluid, or cerebrospinal fluid).

In a particular exemplary aspect, subjects may undergo leukapheresis, wherein leukocytes are collected, enriched, or depleted ex vivo to select and/or isolate the cells of interest, e.g., T cells. These T cell isolates can be expanded by contact with an artificial APC, e.g., an aAPC expressing anti-CD28 and anti-CD3 CD Rs, and treated such that one or more CAR constructs of the technology may be introduced, thereby creating a CAR T cell. Subjects in need thereof can subsequently undergo standard treatment with high dose chemotherapy followed by peripheral blood stem cell transplantation.

Following or concurrent with the transplant, subjects can receive an infusion of the expanded CAR T cells. In some embodiment, expanded cells are administered before or following surgery. In some embodiments, lymphodepletion is performed on a subject prior to administering one or more CART cell as described herein. In such embodiments, the lymphodepletion can include administering one or more of melphalan, cytoxan, cyclophosphamide, and fludarabine. The dosage of the above treatments to be administered to a patient will vary with the precise nature of the condition being treated and the recipient of the treatment. The scaling of dosages for human administration can be performed according to art-accepted practices.

In some embodiments, a single treatment regimen is required. In others, administration of one or more subsequent doses or treatment regimens can be performed. For example, after treatment biweekly for three months, treatment can be repeated once per month, for six months or a year or longer. In some embodiments, no additional treatments are administered following the initial treatment.

The dosage of a composition as described herein can be determined by a physician and

adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

The term “statistically significant” or “significantly” refers to statistical significance and generally means a two standard deviation (2SO) or greater difference. Other than in the operating examples, or where otherwise indicated, all numbers expressing quantities of ingredients or reaction conditions used herein should be understood as modified in all instances by the term “about.”

The term “about” when used in connection with percentages can mean±1%.

As used herein, a “subject” means a human or animal. Usually the animal is a vertebrate such as a primate, rodent, domestic animal or game animal. Primates include, for example, chimpanzees, cynomolgus monkeys, spider monkeys, and macaques, e.g., rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient,” and “subject” are used interchangeably herein. Preferably, the subject is a mammal. The mammal can be a human, non-human primate, mouse, rat, dog, cat, horse, or cow, but is not limited to these examples. Mammals other than humans can be advantageously used as subjects that represent animal models of disease, e.g., cancer. A subject can be male or female.

A subject can be one who has been previously diagnosed with or identified as suffering from or having a condition in need of treatment (e.g., a pancreatic cancer, a lung cancer, an ovarian cancer, endometrial cancer, biliary cancer, gastric cancer, or mesothelioma or another type of cancer expressing CD19, CD37, CEA, CD79, gp100, EGFR, FR-α, HER2, EpHA2, Glypican-3, EGFR806, PSCA, IL-13Ra2, MUC1, MUC16, MAGE-A1/3/4, LMP1, CD171, Claudin 18.2, DR5, EpCAM, DLL-3, GD2, c-MET, VEGFR2, AFP, Nectin4/FAP, EGFRvIII, CD20, Lewis Y, CD22, BCMA, PSMA, AXL, CD80/86, GPRC5D, or mesothelin, among others) or one or more complications related to such a condition, and optionally, have already undergone treatment for the condition or the one or more complications related to the condition.

Alternatively, a subject can also be one who has not been previously diagnosed as having such condition or related complications. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition. As used herein, the term “pharmaceutical composition” refers to the active agent in combination with a pharmaceutically acceptable carrier e.g., a carrier commonly used in the pharmaceutical industry.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier in which the active ingredient would not be found to occur in nature.

In one aspect, the technology described herein relates to a pharmaceutical composition including activated CART cells as described herein, and optionally a pharmaceutically acceptable carrier. The active ingredients of the pharmaceutical composition at a minimum include activated CART cells as described herein. In some embodiments, the active ingredients of the pharmaceutical composition consist essentially of activated CART cells as described herein. In some embodiments, the active ingredients of the pharmaceutical composition consist of activated CART cells as described herein. Pharmaceutically acceptable carriers for cell-based therapeutic formulation include saline and aqueous buffer solutions, Ringer's solution, and serum component, such as serum albumin, HDL and LDL. The terms such as “excipient,” “carrier,” “pharmaceutically acceptable carrier”, “pharmaceutically acceptable excipient” or the like are used interchangeably herein.

In some embodiments, the pharmaceutical composition including activated CART cells as described herein can be a parenteral dose form. Since administration of parenteral dosage forms typically bypasses the patient's natural defenses against contaminants, the components apart from the CART cells themselves are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. Any of these can be added to the activated CART cells preparation prior to administration. Suitable vehicles that can be used to provide parenteral dosage forms of activated CART cells as disclosed within are well known to those skilled in the art. Examples include, without limitation: saline solution; glucose solution; aqueous vehicles including but not limited to, sodium chloride injection, Ringer's injection, dextrose injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

“Unit dosage form” as the term is used herein refers to a dosage for suitable one administration. By way of example, a unit dosage form can be an amount of therapeutic disposed in a delivery device, e.g., a syringe or intravenous drip bag. In some embodiments, a unit dosage form is administered in a single administration. In another, embodiment more than one unit dosage form can be administered simultaneously.

In some embodiments, the activated CART cells described herein are administered as a monotherapy, i.e., another treatment for the condition is not concurrently administered to the subject. A pharmaceutical composition including the T cells described herein can generally be administered at a dosage of 10⁴ to 10⁹ cells/kg body weight, in some instances 10⁵ to 10⁶ cells/kg body weight, including all integer values within those ranges. If necessary, T cell compositions can also be administered multiple times at these dosages. The cells can be administered by using infusion techniques that are commonly known in immunotherapy (see, e.g., Rosenberg et al., New Eng. J. Med. 30 319:1676, 1988).

In certain aspects, it may be desired to administer activated CART cells to a subject and then subsequently redraw blood (or have an apheresis performed), activate T cells therefrom as described herein, and reinfuse the patient with these activated and expanded T cells. This process can be carried out multiple times every few weeks. In certain aspects, T cells can be activated from blood draws of from 10 cc to 400 cc. In certain aspects, T cells are activated from blood draws of 20 cc, 30 cc, 40 cc, 50 cc, 60 cc, 70 cc, 80 cc, 90 cc, or 100 cc.

In some embodiments, the immune cells (e.g., T cells) including a CAR, such as a CART with an IL7-degron described herein, can also be used to treat a cancer having heterogeneous antigen expression. For example, the CAR component of the CART with an IL7-degron construct can include an extracellular target binding domain that binds to one antigen expressed by the cancer, while the TEAM (T cell engaging antibody molecule) component of the CART with an IL7-degron construct can bind a second antigen expressed by the cancer in addition to a T cell antigen (e.g., CD3). “Cancer” as used herein can refer to a hyperproliferation of cells whose unique trait, loss of normal cellular control, results in unregulated growth, lack of differentiation, local tissue invasion, and metastasis. Exemplary cancers include, but are not limited to, glioblastoma, prostate cancer, glioma, leukemia, lymphoma, multiple myeloma, or a solid tumor, e.g., lung cancer and pancreatic cancer. Nonlimiting examples of leukemia include acute myeloid leukemia (AML), chronic myeloid leukemia (CML), acute lymphocytic leukemia (ALL), and chronic lymphocytic leukemia (CLL). In some embodiments, the cancer is ALL or CLL. Non-limiting examples of lymphoma include diffuse large B-cell lymphoma (DLBCL), follicular lymphoma, small lymphocytic lymphoma (SLL), mantle cell lymphoma (MCL), marginal zone lymphomas, Burkitt's lymphoma, hairy cell leukemia (HCL), and T cell lymphoma (e.g., peripheral T cell lymphoma (PTCL), including cutaneous T cell lymphoma (CTCL) and anaplastic large cell lymphoma (ALCL)). In some embodiments, the cancer is DLBCL or follicular lymphoma.

Non-limiting examples of solid tumors include adrenocortical tumor, alveolar soft part sarcoma, carcinoma, chondrosarcoma, colorectal carcinoma, desmoid tumors, desmoplastic small round cell tumor, endocrine tumors, endodermal sinus tumor, epithelioid hemangioendothelioma, Ewing sarcoma, germ cell tumors (solid tumor), giant cell tumor of bone and soft tissue, hepatoblastoma, hepatocellular carcinoma, melanoma, nephroma, neuroblastoma, non-rhabdomyosarcoma soft tissue sarcoma (NRSTS), osteosarcoma, paraspinal sarcoma, renal cell carcinoma, retinoblastoma, rhabdomyosarcoma, synovial sarcoma, and Wilms tumor.

Solid tumors can be found in bones, muscles, or organs, and can be sarcomas or carcinomas. It is contemplated that any aspect of the technology described herein can be used to treat all types of cancers, including cancers not listed in the instant application. As used herein, the term “tumor” refers to an abnormal growth of cells or tissues, e.g., of malignant type or benign type.

Cluster of differentiation (CD) molecules are cell surface markers present on leukocytes. As a leukocyte differentiates and matures its CD profile changes. In the case that a leukocytes turns into a cancer cell (i.e., a lymphoma), its CD profile is important in diagnosing the disease. The treatment and prognosis of certain types of cancers is reliant on determining the CD profile of the cancer cell. “COX+”, wherein “X” is a CD marker, indicates the CD marker is present in the cancer cell, while “COX−” indicates the marker is not present. One skilled in the art will be capable of assessing the CD molecules present on a cancer cell using standard techniques, for example, using immunofluorescence to detect commercially available antibodies bound to the CD molecules.

As used herein, an “autoimmune disease or disorder” is characterized by the inability of one's immune system to distinguish between a foreign cell and a healthy cell. This results in one's immune system targeting one's healthy cells for programmed cell death. Non-limiting examples of an autoimmune disease or disorder include inflammatory arthritis, type 1 diabetes mellitus, multiples sclerosis, psoriasis, inflammatory bowel diseases, SLE, and vasculitis, allergic inflammation, such as allergic asthma, atopic dermatitis, and contact hypersensitivity. Other examples of auto-immune-related disease or disorder, but should not be construed to be limited to, include rheumatoid arthritis, multiple sclerosis (MS), systemic lupus erythematosus, Graves' disease (overactive thyroid), Hashimoto's thyroiditis (underactive thyroid), celiac disease, Crohn's disease and ulcerative colitis, Guillain-Barre syndrome, primary biliary sclerosis/cirrhosis, sclerosing cholangitis, autoimmune hepatitis, Raynaud's phenomenon, scleroderma, Sjogren's syndrome, Goodpasture's syndrome, Wegener's granulomatosis, polymyalgia rheumatica, temporal arteritis/giant cell arteritis, chronic fatigue syndrome CFS), psoriasis, autoimmune Addison's Disease, ankylosing spondylitis, acute disseminated encephalomyelitis, antiphospholipid antibody syndrome, aplastic anemia, idiopathic thrombocytopenic purpura, myasthenia gravis, opsoclonus myoclonus syndrome, optic neuritis, Ord's thyroiditis, pemphigus, pernicious anemia, polyarthritis in dogs, Reiter's syndrome, Takayasu's arteritis, warm autoimmune hemolytic anemia, Wegener's granulomatosis and fibromyalgia (FM). In some embodiments, the mammalian cell is obtained for a patient having an immune system disorder that results in abnormally low activity of the immune system, or immune deficiency disorders, which hinders one's ability to fight a foreign agent (e.g., a virus or bacterial cell). A plasma cell is a white blood cell produces from B lymphocytes which function to generate and release antibodies needed to fight infections. As used herein, a “plasma cell disorder or disease” is characterized by abnormal multiplication of a plasma cell. Abnormal plasma cells are capable of “crowding out” healthy plasma cells, which results in a decreased capacity to fight a foreign object, such as a virus or bacterial cell. Non-limiting examples of plasma cell disorders include amyloidosis, Waldenstrom's macroglobulinemia, osteosclerotic myeloma (POEMS syndrome), monoclonal gammopathy of unknown significance (MGUS), and plasma cell myeloma.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### T Cell Factors

The disclosure provides, in part, T cell factors. A T cell factor comprises a polypeptide that alters T cell (e.g., CAR T cell) differentiation and/or phenotype, activates T cells, increases T cell persistence, and/or increases T cell expansion in a manner that can be controlled. A T cell factor comprises a cytokine domain and a degron domain. The cytokine domain is capable of providing a signal to a T cell, e.g., a CAR T cell, that alters T cell (e.g., CAR T cell) differentiation and/or phenotype, activates a T cell, increases T cell persistence, and/or increases T cell expansion. The degron domain is capable of promoting the degradation of the T cell factor, e.g., by binding to a component of the ubiquitin proteasome pathway or promoting ubiquitination of the T cell factor, in an inducible manner (e.g., a drug-responsive manner). In some embodiments, the T cell factor.

In some embodiments, a T cell factor is drug-responsive, meaning that the T cell altering effects of the T cell factor may be modulated by the presence, absence, or level of a drug. In some embodiments, a T cell factor is inhibited by the presence of a drug. In some embodiments, the T cell altering effects of the T cell factor are negatively correlated with the concentration of a drug. In some embodiments, the presence of a drug promotes degradation of a T cell factor. In some embodiments, the level of the T cell factor in a cell or on the surface of a cell is negatively correlated with the concentration of a drug.

In some embodiments, a T cell factor comprises a membrane-bound polypeptide. In some embodiments, the T cell factor comprises a transmembrane or hinge domain that associates and/or stabilizes the T cell factor in a membrane of a cell, e.g., the cell membrane. In some embodiments, the transmembrane or hinge domain connects an extracellular portion of the T cell factor (e.g., a cytokine domain) to an intracellular portion of the T cell factor (e.g., a degron domain). The transmembrane or hinge domain may be any transmembrane or hinge domain described herein (e.g., described herein for use in a CAR). In some embodiments, the transmembrane or hinge domain is a CD80 transmembrane domain or a functional portion thereof. In some embodiments, the transmembrane or hinge domain comprises an amino acid sequence of SEQ ID NO: 1, or a variant thereof (e.g., a sequence having at least 70%, at least 75%, at least 80%, at least 85%, at least 90%, at least 95%, or at least 99% identity thereto).

(SEQ ID NO: 1)
WNTTKQEHFPDNLLPSWATTLISVNGIFVICCLTYCFAPRCRERRRNER
LRRESVRPV

In some embodiments, the T cell factor comprises a polypeptide wherein the transmembrane or hinge domain is situated between the cytokine domain and the degron domain. In some embodiments, the T cell factor comprises a polypeptide comprising, from N-terminal-most to C-terminal-most, a cytokine domain, a transmembrane or hinge domain, and a degron domain. In some embodiments, the T cell factor comprises a polypeptide comprising, from C-terminal-most to N-terminal-most, a cytokine domain, a transmembrane or hinge domain, and a degron domain. In some embodiments, the cytokine domain is extracellular facing or lumen facing. In some embodiments, the degron domain is intracellular facing or cytosol facing.

In some embodiments, a T cell factor comprises one or more linker domains. As used herein, “linker domain” refers to an oligo- or polypeptide region from about 2 to 100 amino acids in length, which links together any of the domains/regions of the T cell factor as described herein. In some embodiments, a T cell factor comprises a linker domain between the cytokine domain and the transmembrane or hinge domain. In some embodiments, a T cell factor comprises a linker domain between the degron domain and the transmembrane or hinge domain. In some embodiments, a T cell factor comprises a linker domain between the cytokine domain and the degron domain. The linker domains and features of the linker domains that may be used in a CAR of the disclosure are also suitable for use in the linker domains of the T cell factors of the disclosure, and are discussed below.

### Cytokine Domains

Cytokine expression patterns influence T cell differentiation and phenotype before and after activation. The disclosure is directed, in part, to the discovery that a T cell (e.g., a CART cell) expressing a T cell factor comprising a cytokine domain can exhibit improved activation and persistence in vitro and in vivo (e.g., in a subject) relative to a T cell lacking expression of the T cell factor. Without wishing to be bound by theory, by supplying a cytokine, e.g., on the surface of a T cell, said cytokine may activate signaling pathways of either or both of the T cell on which it is expressed and other T cells. The generation of canonical T cell phenotypes (e.g., Th1 and Th2) from naïve CD4+ T cells using cytokines or patterns of cytokines, and thus the roles and properties of different cytokines, is known in the art. See, e.g., Barberis et al. Front. Physiol., 2 Aug. 2018|/doi.org/10.3389/fphys.2018.00879, which is hereby incorporated by reference.

In some embodiments, a cytokine domain comprises or is a polypeptide. In some embodiments, the cytokine domain comprises a recombinant polypeptide. In some embodiments, a cytokine domain comprises a cytokine or functional portion thereof. In some embodiments, the cytokine or functional portion thereof is a human cytokine. In some embodiments, the cytokine domain has 1, 2, 3, 4, 5, 6, or all of the following effects (e.g., when present as part of a T cell factor described herein): promotes proliferation of a T cell; promotes homeostasis of a T cell; acts as an anti-apoptotic signal for a T cell; promotes homeostatic expansion of a T cell; promotes Th1, Th2, or Th17 differentiation of a T cell; activates phosphoinositide 3-kinase (PI3K) signaling and/or Janus kinase (JAK)-signal transducer and activator of transcription (STAT) pathway signaling; and activates STAT5.

In some embodiments, a cytokine domain promotes differentiation of a naïve T cell into an effector T cell phenotype. In some embodiments, a cytokine domain promotes differentiation of a naïve T cell into a memory T cell phenotype. In some embodiments, a cytokine domain promotes differentiation of a naïve T cell into a helper T cell phenotype. In some embodiments, a cytokine domain promotes differentiation of a naïve T cell into a Th1 phenotype. In some embodiments, a cytokine domain promotes differentiation of a naïve T cell into a Th2 phenotype. In some embodiments, a cytokine domain promotes differentiation of a naïve T cell into a Th17 phenotype. In some embodiments, a cytokine domain promotes differentiation of a naïve T cell into a plurality of phenotypes, e.g., 2 or 3 of helper, memory, or effector phenotypes. In some embodiments, a cytokine domain promotes differentiation of a naïve T cell into a plurality of phenotypes, e.g., 2 or 3 of Th1, Th2, or Th17 phenotypes. In some embodiments, a cytokine domain promotes differentiation of a naïve T cell without preference for a differentiated phenotype. For example, the presence of the cytokine domain may induce signaling that promotes differentiation of a naïve T cell generally, without increasing differentiation into a particular phenotype relative to the other phenotypes.

In some embodiments, a cytokine domain comprises an IL-2 family cytokine or portion thereof. In some embodiments, the cytokine domain comprises IL-1, IL-2, IL-4, IL-6, IL-7, IL-10, IL-12, IL-9, IL-15, IL-18, IL-21, or a portion of any thereof. In some embodiments, the cytokine domain comprises IL-2, IL-7, IL-12, IL-18, or a portion of any thereof. In some embodiments, the cytokine domain comprises IL-2 or a portion thereof. In some embodiments, the cytokine domain comprises IL-4 or a portion thereof. In some embodiments, the cytokine domain comprises IL-6 or a portion thereof. In some embodiments, the cytokine domain comprises IL-7 or a portion thereof. In some embodiments, the cytokine domain comprises IL-9 or a portion thereof. In some embodiments, the cytokine domain comprises IL-10 or a portion thereof. In some embodiments, the cytokine domain comprises IL-12 or a portion thereof. In some embodiments, the cytokine domain comprises IL-15 or a portion thereof. In some embodiments, the cytokine domain comprises IL-18 or a portion thereof. In some embodiments, the cytokine domain comprises IL-21 or a portion thereof.

In some embodiments, a cytokine domain comprises an interferon (IFN) family cytokine or portion thereof. In some embodiments, the cytokine domain comprises IFNγ or a portion thereof. In some embodiments, a cytokine domain comprises granulocyte colony-stimulating factor (G-CSF) or portion thereof.

In some embodiments, the cytokine domain is selected from the group consisting of any one of IL-7 or a portion thereof, IL-18 or a portion thereof, and IL-12 or a portion thereof. In some embodiments, the cytokine domain comprises an amino acid sequence selected from any one of SEQ ID NOs: 2-5 or a variant thereof (e.g., an amino acid sequence having at least 70%, at least 75%, at least 80%, at least 85%, at least 90%, at least 95%, or at least 99% identity to any thereof, or an amino acid sequence having ≤1, ≤2, ≤3, ≤4, ≤5, ≤6, ≤7, ≤8, ≤9, or ≤10 substitutions relative to any thereof).

In some embodiments, the cytokine domain comprises an amino acid of SEQ ID NO: 3 or a variant thereof (e.g., an amino acid sequence having at least 70%, at least 75%, at least 80%, at least 85%, at least 90%, at least 95%, or at least 99% identity to any thereof; or an amino acid sequence having 1, ≤2≤3≤4≤5≤6≤7≤8≤9, or ≤10 substitutions relative to any thereof). In some embodiments, the cytokine domain comprises SEQ ID NO: 3. In some embodiments, the cytokine domain consists of SEQ ID NO: 3. Exemplary cytokine domain amino acid sequences can be found in Table 1.

TABLE 1
Exemplary Cytokine Domain Sequences

| SEQ | | |
|---|---|---|
| ID | | |
| NO | Description | Sequence |
| 2 | IL-7 full | MFHVSFRYIFGLPPLILVLLPVASSDCDIEGKDGKQYESV |
| | | LMVSIDQLLDSMKEIGSNCLNNEFNFFKRHICDANKEGMF |
| | | LFRAARKLRQFLKMNSTGDFDLHLLKVSEGTTILLNCTGQ |
| | | VKGRKPAALGEAQPTKSLEENKSLKEQKKLNDLCFLKRLL |
| | | QEIKTCWNKILMGTKEH |
| 3 | IL-7 | DCDIEGKDGKQYESVLMVSIDQLLDSMKEIGSNCLNNEFNFFKRHICD |
| | portion | ANKEGMFLFRAARKLRQFLKMNSTGDFDLHLLKVSEGTTILLNCTGQV |
| | | KGRKPAALGEAQPTKSLEENKSLKEQKKLNDLCFLKRLLQEIKTCWNK |
| | | ILMGTKEH |
| 4 | IL-18 | YFGKLESKLSVIRNLNDQVLFIDQGNRPLFEDMTDSDCRDNAPRTIFI |
| | | ISMYKDSQPRGMAVTISVKCEKISTLSCENKIISFKEMNPPDNIKDTK |
| | | SDIIFFQRSVPGHDNKMQFESSSYEGYFLACEKERDLFKLILKKEDEL |
| | | GDRSIMFTVQNED |
| 5 | IL-12 (p40, | MCHQQLVISWFSLVFLASPLVAIWELKKDVYVVELDWYPDAPGEMVVL |
| | (G4S)3, | TCDTPEEDGITWTLDQSSEVLGSGKTLTIQVKEFGDAGQYTCHKGGEV |
| | p35) | LSHSLLLLHKKEDGIWSTDILKDQKEPKNKTFLRCEAKNYSGRFTCWW |
| | | LTTISTDLTFSVKSSRGSSDPQGVTCGAATLSAERVRGDNKEYEYSVE |
| | | CQEDSACPAAEESLPIEVMVDAVHKLKYENYTSSFFIRDIIKPDPPKN |
| | | LQLKPLKNSRQVEVSWEYPDTWSTPHSYFSLTFCVQVQGKSKREKKDR |
| | | VFTDKTSATVICRKNASISVRAQDRYYSSSWSEWASVPCSGGGGSGGG |
| | | GSGGGGSMCPARSLLLVATLVLLDHLSLARNLPVATPDPGMFPCLHHS |
| | | QNLLRAVSNMLQKARQTLEFYPCTSEEIDHEDITKDKTSTVEACLPLE |
| | | LTKNESCLNSRETSFITNGSCLASRKTSFMMALCLSSIYEDLKMYQVE |
| | | FKTMNAKLLMDPKRQIFLDQNMLAVIDELMQALNENSETVPQKSSLEE |
| | | PDFYKTKIKLCILLHAFRIRAVTIDRVMSYLNAS |

### Degron Domains

In some embodiments, a degron domain promotes degradation of the degron and any operably linked molecule (e.g., a polypeptide comprising the degron domain) when a 15 condition is met. In some embodiments, the condition is the presence of a drug. In some embodiments, the condition is the presence of a level of a drug above a reference concentration. In some embodiments, the reference concentration can be achieved in vivo, e.g., in a subject, by administering the drug as described herein, e.g., at a sub-therapeutic dose for the drug.

In some embodiments, a degron domain comprises or is a polypeptide. In some embodiments, a degron domain comprises a C2H2 zinc finger degron motif. In some embodiments, a degron domain comprises a cereblon (CRBN) polypeptide substrate domain. CRBN is an E3 ubiquitin ligase complex comprising damaged DNA binding protein 1 (DDB1), Cullin-4A (CUL4) and regulator of cullins 1 (ROC1). CRBN binds to and ubiquitinates CRBN polypeptide substrates, which in turn are recognized and degraded by the proteasome. CRBN polypeptide substrates comprise recurrent Cys2-His2 (C2H2) zinc finger motifs. Thalidomide analogs, e.g., thalidomide, lenalidomide, pomalidomide and analogs thereof (e.g., immunomodulatory imide drugs (ImiDs)), are known to bind to C2H2 zinc finger-containing proteins and induce degradation of those proteins. Previous work has shown that C2H2 zinc finger containing proteins can be engineered to create drug-responsive CRBN polypeptide substrate domains that bind thalidomide analogs. As used herein, a CRBN polypeptide substrate domain refers to a polypeptide comprising at least one C2H2 zinc finger motif and that is drug-inducible a substrate for CRBN. In some embodiments, a CRBN polypeptide substrate domain is a recombinant polypeptide (e.g., is a non-naturally occurring fragment of a naturally occurring CRBN polypeptide substrate). In some embodiments, a CRBN polypeptide substrate domain is a degron domain of a T cell factor and is heterologous to another domain of the T cell factor, to the cell in which the T cell factor is comprised, or both.

Without wishing to be bound by theory, when bound to an thalidomide analog, a CRBN polypeptide substrate domain is a substrate for CRBN; when not bound to an thalidomide analog a CRBN polypeptide substrate domain is not a substrate for CRBN or is a poor substrate for CRBN. Without wishing to be bound by theory, a thalidomide analog is thought to mediate formation of a ternary CRBN:thalidomide analog:CRBN polypeptide substrate domain complex, which results in ubiquitination of the CRBN polypeptide substrate domain. The disclosure is directed, in part, to the idea of controlling ubiquitination (e.g., by CRBN) of a T cell factor comprising a CRBN polypeptide substrate domain by contacting the T cell factor with a thalidomide analog.

In some aspects, the degron domain comprises a CRBN polypeptide substrate domain. CRBN polypeptide substrates and C2H2 zinc finger containing-polypeptides are well known in the art (e.g., as described in PCT patent publication WO2019089592, which is hereby incorporated by reference in its entirety). In some embodiments, the CRBN polypeptide substrate domain comprises a naturally occurring CRBN polypeptide substrate or portion thereof capable of inducing degradation of a polypeptide (e.g., a T cell factor) in which it is comprised. In some embodiments, the CRBN polypeptide substrate domain comprises a CRBN polypeptide substrate selected from the group consisting of IKZF1, IKZF3, CK1α, ZFP91, GSPT1, MEIS2, GSS, E4F1, ZN276, ZN517, ZN582, ZN653, ZN654, ZN692, ZN787, ZN827, or a fragment thereof that is capable of drug-inducibly binding CRBN (e.g., SEQ ID NOs: 6-22).

As used herein, “drug-inducible” refers to a condition or event (a) the occurrence of which is dependent upon the presence of a drug (e.g., a thalidomide analog); in other words, the condition or event does not occur in the absence of the drug, or (b) the occurrence of which (e.g., frequency of occurrence) is increased by the presence of a drug (e.g., a thalidomide analog). For example, in some embodiments, binding of CRBN to a CRBN polypeptide substrate domain is drug-inducible. In some embodiments, ubiquitination activity of CRBN on a CRBN polypeptide substrate domain is drug-inducible. As used herein, “drug-responsive” is a characteristic of a composition (e.g., a polypeptide (e.g., a T cell factor) or a cell (e.g., a mammalian cell, e.g., a T cell, e.g., a CAR T cell)) which is altered in response to the presence of a drug. In some embodiments, a drug-responsive composition acts or participates in a process or is subject to an action or process in response to the presence of a drug. In some embodiments, a drug-responsive composition has one or more altered characteristics in response to the presence of a drug. For example, a T cell factor that is drug-responsive may comprise a degron domain and be subject to ubiquitination and degradation in the presence of a drug. As a further example, a mammalian cell (e.g., a CAR T cell) that is drug-responsive may be less active, less proliferative, and/or persist for a shorter time in a subject in the presence of the drug.

In some embodiments, the CRBN polypeptide substrate domain comprises a CRBN polypeptide substrate selected from the group consisting of IKF1, IKZF3, and CK1-alpha. In some embodiments, the CRBN polypeptide substrate domain comprises IKZF3 or a fragment thereof. In some embodiments, the IKZF3 or fragments thereof are capable of drug-inducible binding CRBN. In some embodiments, the CRBN polypeptide substrate domain comprises amino acids 130-145, amino acids 169-189, or amino acids 130-189 of IKZF3.

In some embodiments, the CRBN polypeptide substrate domain is a hybrid fusion polypeptide comprising a fragment of a first CRBN polypeptide substrate and a fragment of a second CRBN polypeptide substrate (e.g., a chimera of IKZF3 and CK1-alpha). In some embodiments, the hybrid fusion polypeptide comprising the N-terminal beta hairpin of a first CRBN polypeptide substrate and the c-terminal alpha-helix of a second CRBN polypeptide substrate. In some embodiments, the hybrid fusion polypeptide is 18, 19, 20, 21, 22, 23, 24, 25, 26, 27, 28, 29, 30, 31, 32, 33, 34, 35, 36, 37, 38, 39 or 40 amino acids in length. In some embodiments, the hybrid fusion polypeptide comprises the N-terminal portion of any one of ZN653, ZN827, ZFP91, ZN276, IKZF3 and the C-terminal portion of any one of ZN787, ZN517, IKZF3, ZN654, PATZ1, E4F1, and ZKSC5, as described in Jan et al., Sci Transl Med. 2021 Jan. 6; 13(575): eabb6295. doi: 10.1126/scitranslmed.abb6295, which is hereby incorporated by reference in its entirety. In some embodiments, the hybrid fusion polypeptide is a super-degron as described in Jan et al., Sci Transl Med. 2021 Jan. 6; 13(575): eabb6295. doi: 10.1126/scitranslmed.abb6295.

In some embodiments, the CRBN polypeptide substrate domain comprises a hybrid fusion polypeptide comprised of ten or more residues of a non-IKZF3 C2H2 zinc finger degron sequence flanked by an N-terminal IKZF3 degron sequence and a C-terminal IKZF3 degron sequence.

In some embodiments, the N-terminal IKZF3 degron sequence comprises or is amino acids 130-145 (SEQ ID NO: 23) of IKZF3 and/or the C-terminal IKZF3 degron sequence comprises or is amino acids 169-189 (SEQ ID NO: 25) of IKZF3.

In some embodiments, the CRBN polypeptide substrate domain is SEQ ID NO: 27.

In some embodiments, the non-IKZF3 C2H2 zinc finger degron sequence is a ZFP91 sequence, e.g., amino acids 400-422 of ZFP91.

Any drug that induces binding and/or activity of CRBN on the CRBN polypeptide substrate domain is suitable for use in the methods and compositions described herein. In some embodiments, the drug is a thalidomide analog. In some embodiments, the drug is a immunomodulatory imide drug (IMiD). In some embodiments, the drug is a small molecule drug. In some embodiments, the drug is an FDA-approved drug. In some embodiments, the drug is selected from the group consisting of thalidomide, lenalidomide, avadomide, iberdomide, and pomalidomide. In some embodiments, the drug is thalidomide. In some embodiments, the drug is lenalidomide. In some embodiments, the drug is pomalidomide. In some embodiments, the drug can be administered to a human subject in a clinical setting using a method of administration described herein. In some embodiments, the drug is associated with a therapeutic effect, e.g., the treatment of a disease or condition. In some embodiments, the therapeutic effect is not related to a disease or condition that is treatable or treated by a T cell comprising the T cell factor (and optionally a CAR). In some embodiments, the therapeutic effect is related to a disease or condition that is treatable or treated by a T cell comprising the T cell factor (and optionally a CAR). In some embodiments, the drug can induce CRBN binding and/or activity on the CRBN polypeptide substrate domain at a sub-therapeutic dose (i.e., a level that is less than the level produced by a prescribed dosage or recommended/required for the therapeutic effect). For example, a drug, e.g., lenalidomide, may have a therapeutic effect (e.g., treatment of multiple myeloma) and drug-inducible binding of CRBN to CRBN polypeptide substrate domain may be achieved at a dose lower than the dose required or prescribed to treat, e.g., multiple myeloma.

In some embodiments, the degron domain comprises a CRBN polypeptide substrate domain comprising IKZF1, IKZF3, CK1α, ZFP91, GSPT1, MEIS2, GSS, E4F1, ZN276, ZN517, ZN582, ZN653, ZN654, ZN692, ZN787, ZN827, or a fragment thereof that is capable of drug-inducibly binding CRBN. In some embodiments, the degron domain comprises an amino acid sequence selected from any one of SEQ ID NOs: 6-23, 25, 27, 28, or a variant thereof (e.g., an amino acid sequence having at least 70%, at least 75%, at least 80%, at least 85%, at least 90%, at least 95%, or at least 99% identity to any thereof, or an amino acid sequence having ≤1, ≤2, ≤3, ≤4, ≤5, ≤6, ≤7, ≤8, ≤9, or ≤10 substitutions relative to any thereof).

In some embodiments, the degron domain comprises an amino acid of SEQ ID NO: 22 or a variant thereof (e.g., an amino acid sequence having at least 70%, at least 75%, at least 80%, at least 85%, at least 90%, at least 95%, or at least 99% identity to any thereof, or an amino acid sequence having ≤1, ≤2, ≤3, ≤4, ≤5, ≤6, ≤7, ≤8, ≤9, or ≤10 substitutions relative to any thereof). In some embodiments, the degron domain comprises the amino acid sequence of SEQ ID NO: 22. In some embodiments, the degron domain consists of SEQ ID NO: 22.

Exemplary degron domain amino acid sequences can be found in Table 2.

TABLE 2
Exemplary Degron Domain Sequences

| SEQ | | |
|---|---|---|
| ID | | |
| NO | Description | Sequence |
| 6 | IKZF1 | MDADEGQDMSQVSGKESPPVSDTPDEGDEPMPIPEDLSTTSGGQQSSK |
| | | SDRVVASNVKVETQSDEENGRACEMNGEECAEDLRMLDASGEKMNGSH |
| | | RDQGSSALSGVGGIRLPNGKLKCDICGIICIGPNVLMVHKRSHTGERP |
| | | FQCNQCGASFTQKGNLLRHIKLHSGEKPFKCHLCNYACRRRDALTGHL |
| | | RTHSVGKPHKCGYCGRSYKQRSSLEEHKERCHNYLESMGLPGTLYPVI |
| | | KEETNHSEMAEDLCKIGSERSLVLDRLASNVAKRKSSMPQKFLGDKGL |
| | | SDTPYDSSASYEKENEMMKSHVMDQAINNAINYLGAESLRPLVQTPPG |
| | | GSEVVPVISPMYQLHKPLAEGTPRSNHSAQDSAVENLLLLSKAKLVPS |
| | | EREASPSNSCQDSTDTESNNEEQRSGLIYLTNHIAPHARNGLSLKEEH |
| | | RAYDLLRAASENSQDALRVVSTSGEQMKVYKCEHCRVLFLDHVMYTIH |
| | | MGCHGFRDPFECNMCGYHSQDRYEFSSHITRGEHRFHMS |
| 7 | CKla | MASSSGSKAEFIVGGKYKLVRKIGSGSFGDIYLAINITNGEEVAVKLE |
| | | SQKARHPQLLYESKLYKILQGGVGIPHIRWYGQEKDYNVLVMDLLGPS |
| | | LEDLFNFCSRRFTMKTVLMLADQMISRIEYVHTKNFIHRDIKPDNFLM |
| | | GIGRHCNKLFLIDFGLAKKYRDNRTRQHIPYREDKNLTGTARYASINA |
| | | HLGIEQSRRDDMESLGYVLMYFNRTSLPWQGLKAATKKQKYEKISEKK |
| | | MSTPVEVLCKGFPAEFAMYLNYCRGLRFEEAPDYMYLRQLFRILFRTL |
| | | NHQYDYTFDWTMLKQKAAQQAASSSGQGQQAQTPTGKQTDKTKSNMKG |
| | | F |
| 8 | ZFP91 | MPGETEEPRPPEQQDQEGGEAAKAAPEEPQQRPPEAVAAAPAGTTSSR |
| | | VLRGGRDRGRAAAAAAAAAVSRRRKAEYPRRRRSSPSARPPDVPGQQP |
| | | QAAKSPSPVQGKKSPRLLCIEKVTTDKDPKEEKEEEDDSALPQEVSIA |
| | | ASRPSRGWRSSRTSVSRHRDTENTRSSRSKTGSLQLICKSEPNTDQLD |
| | | YDVGEEHQSPGGISSEEEEEEEEEMLISEEEIPFKDDPRDETYKPHLE |
| | | RETPKPRRKSGKVKEEKEKKEIKVEVEVEVKEEENEIREDEEPPRKRG |
| | | RRRJDDKSPRLPKRRKKPPIQYVRCEMEGCGTVLAHPRYLQHHIKYQH |
| | | LLKKKYVCPHPSCGRLFRLQKQLLRHAKHHTDQRDYICEYCARAFKSS |
| | | HNLAVHRMIHTGEKPLQCEICGFTCRQKASLNWHMKKHDADSFYQFSC |
| | | CGKKFEKKDSVVAHKAKSHPEVLIAEALAANAGALITSTDILGTPESL |
| | | TQPSDGQGLPLLPEPLGNSTSGECLLLEAEGMSKSYCSGTERVSLMAD |
| | | GKIFVGSGSSGGTEGLVMNSDILGATTEVLIEDSDSAGP |
| 9 | GSPT1 | MDPGSGGGGGGGGSSSGSSSSDSAPDCWDQADMEAPGPGPCGGGGSLA |
| | | AAAEAQRENLSAAFSRQLNVNAKPFVPNVHAAEFVPSFLRCPAAPPPP |
| | | AGGAANNHGAGSGAGGRAAPVESSQEEQSLCEGSNSAVSMELSEPIEN |
| | | GETEMSPEESWEHKEEISEAEPGGGSLGDGRPPEESAFIEMMEEEEEI |
| | | PKPKSVVAPPGAPKKEHVNVVFIGHVDAGKSTIGGQFYLTGMVDKRTL |
| | | EKYEREAKEKNRETWYLSWALDTNQEERDKGKTVEVGRAYFETEKKHF |
| | | TILDAPGHKSFVPNMIGGASQADLAVLVISARKGEFETGFEKGGQTRE |
| | | HAMLAKTAGVKHLIVLINKMDDPTVNWSNERYEECKEKLVPFLKKVGF |
| | | NPKKDIHFMPCSGLIGANLKEQSDFCPWYIGLPFIPYLDNLPNFNRSV |
| | | DGPIRLPIVDKYKDMGTVVLGKLESGSICKGQQLVMMPNKHNVEVLGI |
| | | LSDDVETDTVAPGENLKIRLKGIEEEEILPGFILCDPNNLCHSGRTED |
| | | AQIVIIEHKSIICPGYNAVLHIHTCIEEVEITALICLVDKKSGEKSKT |
| | | RPRFVKQDQVCIARLRTAGTICLETFKDFPQMGRFTLRDEGKTIAIGK |
| | | VLKLVPEKD |
| 10 | MEIS2 | MAQRYDELPHYGGMDGVGVPASMYGDPHAPRPIPPVHHLNHGPPLHAT |
| | | QHYGAHAPHPNVMPASMGSAVDALKRDKDAIYGHPLFPLLALVFEKCE |
| | | LATCTPREPGVAGGDVCSSDSFEDIAVFAKQVRAEKPLESSPELDM.M |
| | | IQAIQVLRFHLLELEKVHELCDFCHRYISCLKGKMPIDLVIDERDGSS |
| | | KSDHEELSGSSTNLADHNPSSWRDHDDATSTHSAGTPGPSSGGHASQS |
| | | GDNSSEQGDGLDNSVASPGTGDDDDPDKDKKRQKKRGIFPKVATNE |
| | | RAWLFQHLTHPYPSEEQKKQLAQDTGLTILQVNNWFINARRRIVQPMI |
| | | DQSRAGFLLDPSVSQGAAYSPEGQPMGSFVLDGQQHMGIRPAGPMSGM |
| | | GMNMGMDGQWHYM |
| 11 | GSS | MATNWGSLLQDKQQLEELARQAVDRALAEGVLLRTSQEPTSSEWSYAP |
| | | FTLFPSLVPSALLEQAYAVQMDENLLVDAVSQNAAFLEQTLSSTIKQD |
| | | DFTARLFDIHKQVLKEGIAQTVFLGLNRSDYMFQRSADGSPALKQIEI |
| | | NTISASFGGLASRTPAVHRHVLSVLSKTKEAGKILSNNPSKGLALGIA |
| | | KAWELYGSPNALVLLIAQEKERNIFDQRAIENELLARNIHVIRRTFED |
| | | ISE |
| | | KGSLDQDRRLFVDGQEIAVVYFRDGYMPRQYSLQNWEARLLLERSHAA |
| | | KCPDIATQLAGTKKVQQELSRPGMLEMLLPGQPEAVARLRATFAGLYS |
| | | LDVGEEGDQAIAEALAAPSRFVLKPQREGGGNNLYGEEMVQALKQLKD |
| | | SEERASYILMEKIEPEPFENCLLRPGSPARVVQCISELGIFGVYVRQE |
| | | KTLVMNKHVGHLLRTKAIEHADGGVAAGVAVLDNPYPV |
| 12 | E4F1 amino | HECKLCGASFRTKGSLIRHHRRH |
| | acids 220- | |
| | 242 | |
| | sequence | |
| 13 | ZN276 | LQCEVCGFQCRQRASLKYHMTKH |
| | amino | |
| | acids 524- | |
| | 546 | |
| 14 | ZN517 | YRCRACGRACSRLSTLIQHQKVH |
| | amino | |
| | acids 452- | |
| | 474 | |
| 15 | ZN582 | YQCKVCGRAFKRVSHLTVHYRIH |
| | amino | |
| | acids 395- | |
| | 417 | |
| 16 | ZN653 | LQCEICGYQCRQRASLNWHMKKH |
| | amino | |
| | acids 556- | |
| | 578 | |
| 17 | ZN654 | FACVICGRKFRNRGLMQKHLKNH |
| | amino | |
| | acids 25-47 | |
| 18 | ZN692 | LQCEICGFTCRQKASLNWHQRKH |
| | amino | |
| | acids 417- | |
| | 439 | |
| 19 | ZN787 | FVCPRCGRGFSQPKSLARHLRLH |
| | amino | |
| | acids 178- | |
| | 200 | |
| 20 | ZN827 | FQCPICGLVIKRKSYWKRHMVIH |
| | amino | |
| | acids 374- | |
| | 396 | |
| 21 | IKZF3 | MEDIQTNAELKSTQEQSVPAESAAVLNDYSLTKSHEMENVDSGEGPAN |
| | | EDEDIGDDSMKVKDEYSERDENVLKSEPMGNAEEPEIPYSYSREYNEY |
| | | ENIKLERHVVSFDSSRPTSGKMNCDVCGLSCISFNVLMVHKRSHTGER |
| | | PFQCNQCGASFTQKGNLLRHIKLHTGEKPFKCHLCNYACQRRDALTGH |
| | | LRTHSVEKPYKCEFCGRSYKQRSSLEEHKERCRTFLQSTDPGDTASAE |
| | | ARHIKAEMGSERALVLDRLASNVAKRKSSMPQKFIGEKRHCFDVNYNS |
| | | SYMYEKESELIQTRMMDQAINNAISYLGAEALRPLVQTPPAPTSEMVP |
| | | VISSMYPIALTRAEMSNGAPQELEKKSIHLPEKSVPSERGLSPNNSGH |
| | | DSTDTDSNHEERQNHIYQQNHMVLSRARNGMPLLKEVPRSYELLKPPP |
| | | ICPRDSVKVINKEGEVMDVYRCDHCRVLFLDYVMFTIHMGCHGFRDPF |
| | | ECNMCGYRSHDRYEFSSHIARGEHRALLK |
| 22 | IKZF3 | FNVLMVHKRSHTGERPLQCEICGFTCRQKGNLLRHIKLHTGEKPFKCH |
| | amino | LCNYACQRRDAL |
| | acids 130- | |
| | 189 | |
| 23 | KZF3 | FNVLMVHKRSHTGERP |
| | amino | |
| | acids 130- | |
| | 145 | |
| 24 | K0″ form | FNVLMVHRRSHTGERP |
| | of IKZF3 | |
| | amino | |
| | acids 130- | |
| | 145 | |
| 25 | IKZF3 | TGEKPFKCHLCNYACQRRDAL |
| | amino | |
| | acids 169- | |
| | 189 | |
| 26 | (″K0″ form | TGERPFRCHLCNYACQRRDAL |
| | of IKZF3 | |
| 27 | IKZF3 | FNVLMVHKRSHTGERPFQCNQCGASFTQKGNLLRHIKLHTGEKPFKCH |
| | degron | LCNYACQRRDAL |
| | polypeptide | |
| 28 | IKZF3 | GSGSGSGSGGFNVLMVHKRSHTGERPLQCEICGFTCRQKGNLLRHIKL |
| | with linker | HTGEKPFKCHLCNYACQRRDAL |

### Exemplary T Cell Factors

In some embodiments, a T cell factor comprises a degron domain comprising any one of the CRBN polypeptide substrate domain sequences described herein and a cytokine domain described herein. In some embodiments, the CRBN polypeptide substrate domain comprises a CRBN polypeptide substrate selected from the group consisting of IKF1, IKZF3, and CK1-alpha, a functional portion of any thereof, or a hybrid fusion polypeptide comprising a portion of any thereof, and the cytokine domain comprises IL-2, IL-7, IL-15, IL-18 or a functional portion of any thereof. In some embodiments, the CRBN polypeptide substrate domain comprises a CRBN polypeptide substrate selected from the group consisting of IKF1, IKZF3, and CK1-alpha, a functional portion of any thereof, or a hybrid fusion polypeptide comprising a portion of any thereof, and the cytokine domain comprises IL-7 or a functional portion thereof. In some embodiments, the CRBN polypeptide substrate domain comprises a CRBN polypeptide substrate comprising IKZF3, a functional portion thereof, or a hybrid fusion polypeptide comprising a portion of any thereof, and the cytokine domain comprises IL-7 or a functional portion thereof. In some embodiments, the hybrid fusion polypeptide comprises ZFP91 or a portion thereof, e.g., IKZF3 and ZFP91 or portions of either or both thereof. In some embodiments, a T cell factor comprises a transmembrane or hinge domain comprising a CD80 transmembrane domain. In some embodiments, a T cell factor comprises a CRBN polypeptide substrate domain comprising a CRBN polypeptide substrate comprising IKZF3, a functional portion thereof, or a hybrid fusion polypeptide comprising a portion of any thereof, a cytokine domain comprises IL-7 or a functional portion thereof, and a transmembrane or hinge domain comprising a CD80 transmembrane domain.

In some embodiments, a T cell factor comprises a degron domain comprising an amino acid sequence selected from any one of SEQ ID NOs: 6-23, 25, 27, 28 or a variant thereof (e.g., an amino acid sequence having at least 70%, at least 75%, at least 80%, at least 85%, at least 90%, at least 95%, or at least 99% identity to any thereof, or an amino acid sequence having ≤1, ≤2, 3, ≤4, ≤5, ≤6, ≤7, ≤8, ≤9, or ≤10 substitutions relative to any thereof). In some embodiments, a T cell factor comprises a cytokine domain comprising an amino acid sequence selected from any one of SEQ ID NOs: 2-5 or a variant thereof (e.g., an amino acid sequence having at least 70%, at least 75%, at least 80%, at least 85%, at least 90%, at least 95%, or at least 99% identity to any thereof; or an amino acid sequence having ≤1, ≤2, ≤3, ≤4, ≤5, ≤6, ≤7, ≤8, ≤9, or ≤10 substitutions relative to any thereof). In some embodiments, a T cell factor comprises a degron domain comprising an amino acid sequence selected from any one of SEQ ID NOs: 6-23, 25, 27, 28 or a variant thereof (e.g., an amino acid sequence having at least 70%, at least 75%, at least 80%, at least 85%, at least 90%, at least 95%, or at least 99% identity to any thereof, or an amino acid sequence having ≤1, ≤2, ≤3, ≤4, ≤5, ≤6, ≤7, ≤8, ≤9, or ≤10 substitutions relative to any thereof), and a cytokine domain comprising an amino acid sequence selected from any one of SEQ ID NOs: 2-5 or a variant thereof (e.g., an amino acid sequence having at least 70%, at least 75%, at least 80%, at least 85%, at least 90%, at least 95%, or at least 99% identity to any thereof, or an amino acid sequence having ≤1, ≤2, ≤3, ≤4, ≤5, ≤6, ≤7, ≤8, ≤9, or ≤10 substitutions relative to any thereof). In some embodiments, a T cell factor comprises a transmembrane or hinge domain comprising an amino acid sequence selected from any one of SEQ ID NOs: 1, 47-48, or a variant thereof (e.g., an amino acid sequence having at least 70%, at least 75%, at least 80%, at least 85%, at least 90%, at least 95%, or at least 99% identity to any thereof, or an amino acid sequence having ≤1, ≤2, ≤3, ≤4, ≤5, ≤6, ≤7, ≤8, ≤9, or ≤10 substitutions relative to any thereof). In some embodiments, a T cell factor comprises an amino acid sequence selected from of SEQ ID NO: 29 or a variant thereof (e.g., an amino acid sequence having at least 70%, at least 75%, at least 80%, at least 85%, at least 90%, at least 95%, or at least 99% identity to any thereof; or an amino acid sequence having ≤1, ≤2, ≤3, ≤4, ≤5, ≤6, ≤7, ≤8, ≤9, or ≤10 substitutions relative to any thereof).

In some embodiments, any of the exemplary T cell factors described herein are expressed from multicistronic (e.g., bicistronic) vectors, e.g., that also encode a CAR and/or therapeutic agent. In some embodiments, a T cell factor is expressed as part of a polypeptide also comprising a CAR. In some embodiments, the polypeptide comprising the T cell factor and the CAR comprises a cleavable site between the T cell factor and the CAR. In some embodiments, the polypeptide comprising the T cell factor and the CAR comprises an amino acid sequence selected from any one of SEQ ID NOs: 32-37 or a variant thereof (e.g., an amino acid sequence having at least 70%, at least 75%, at least 80%, at least 85%, at least 90%, at least 95%, or at least 99% identity to any thereof; or an amino acid sequence having ≤1, ≤2, ≤3, ≤4, ≤5, ≤6, ≤7, ≤8, ≤9, or ≤10 substitutions relative to any thereof).

TABLE 3
Exemplary T cell factor sequences

| SEQ | | |
|---|---|---|
| ID | | |
| NO | Description | Sequence |
| 29 | IL-7 | MLLLVTSLLLCELPHPAFLLIPEQKLISEEDLDCDIEGKDGKQYESVL |
| | degron | MVSIDQLLDSMKEIGSNCLNNEFNFFKRHICDANKEGMFLFRAARKLR |
| | | QFLKMNSTGDFDLHLLKVSEGTTILLNCTGQVKGRKPAALGEAQPTKS |
| | | LEENKSLKEQKKLNDLCFLKRLLQEIKTCWNKILMGTKEHWNTTKQEH |
| | | FPDNLLPSWAITLISVNGIFVICCLTYCFAPRCRERRRNERLRRESVR |
| | | PVGSGSGSGSGGFNVLMVHKRSHTGERPLQCEICGFTCRQKGNLLRHI |
| | | KLHTGEKPFKCHLCNYACQRRDAL |
| 30 | hGMCSF | MLLLVTSLLLCELPHPAFLLIP |
| | leader | |
| 31 | Myc-tag | EQKLISEEDL |
| 32 | CAR19- | MALPVTALLLPLALLLHAARPEIVMTQSPATLSLSPGERATLSCRASQ |
| | P2A-IL7 | DISKYLNWYQQKPGQAPRLLIYHTSRLHSGIPARFSGSGSGTDYTLTI |
| | degron | SSLQPEDFAVYFCQQGNTLPYTFGQGTKLEIKGGGGSGGGGSGGGGSG |
| | | GGGSQVQLQESGPGLVKPSETLSLTCTVSGVSLPDYGVSWIRQPPGKG |
| | | LEWIGVIWGSETTYYQSSLKSRVTISKDNSKNQVSLKLSSVTAADTAV |
| | | YYCAKHYYYGGSYAMDYWGQGTLVTVSSTTTPAPRPPTPAPTIASQPL |
| | | SLRPEACRPAAGGAVHTRGLDFACDIYIWAPLAGTCGVLLLSLVITLY |
| | | CKRGRKKLLYIFKQPFMRPVQTTQEEDGCSCRFPEEEEGGCELRVKES |
| | | RSADAPAYQQGQNQLYNELNLGRREEYDVLDKRRGRDPEMGGKPRRKN |
| | | PQEGLYNELQKDKMAEAYSEIGMKGERRRGKGHDGLYQGLSTATKDTY |
| | | DALHMQALPPRGSGATNFSLLKQAGDVEENPGPMLLLVTSLLLCELPH |
| | | PAFLLIPEQKLISEEDLDCDIEGKDGKQYESVLMVSIDQLLDSMKEIG |
| | | SNCLNNEFNFFKRHICDANKEGMFLFRAARKLRQFLKMNSTGDFDLHL |
| | | LKVSEGTTILLNCTGQVKGRKPAALGEAQPTKSLEENKSLKEQKKLND |
| | | LCFLKRLLQEIKTCWNKILMGTKEHWNTTKQEHFPDNLLPSWAITLIS |
| | | VNGIFVICCLTYCFAPRCRERRRNERLRRESVRPVGSGSGSGSGGENV |
| | | LMVHKRSHTGERPLQCEICGFTCRQKGNLLRHIKLHTGEKPFKCHLCN |
| | | YACQRRDAL |
| 33 | IL7 | MLLLVTSLLLCELPHPAFLLIPEQKLISEEDLDCDIEGKDGKQYESVL |
| | degron- | MVSIDQLLDSMKEIGSNCLNNEFNFFKRHICDANKEGMELFRAARKLR |
| | P2A- | QFLKMNSTGDFDLHLLKVSEGTTILLNCTGQVKGRKPAALGEAQPTKS |
| | CAR19 | LEENKSLKEQKKLNDLCFLKRLLQEIKTCWNKILMGTKEHWNTTKQEH |
| | | FPDNLLPSWAITLISVNGIFVICCLTYCFAPRCRERRRNERLRRESVR |
| | | PVGSGSGSGSGGFNVLMVHKRSHTGERPLQCEICGFTCRQKGNLLRHI |
| | | KLHTGEKPFKCHLCNYACQRRDALGSGATNFSLLKQAGDVEENPGPMA |
| | | LPVTALLLPLALLLHAARPEIVMTQSPATLSLSPGERATLSCRASQDI |
| | | SKYLNWYQQKPGQAPRLLIYHTSRLHSGIPARFSGSGSGTDYTLTISS |
| | | LQPEDFAVYFCQQGNTLPYTFGQGTKLEIKGGGGSGGGGSGGGGSGGG |
| | | GSQVQLQESGPGLVKPSETLSLTCTVSGVSLPDYGVSWIRQPPGKGLE |
| | | WIGVIWGSETTYYQSSLKSRVTISKDNSKNQVSLKLSSVTAADTAVYY |
| | | CAKHYYYGGSYAMDYWGQGTLVTVSSTTTPAPRPPTPAPTIASQPLSL |
| | | RPEACRPAAGGAVHTRGLDFACDIYIWAPLAGTCGVLLLSLVITLYCK |
| | | RGRKKLLYIFKQPFMRPVQTTQEEDGCSCRFPEEEEGGCELRVKFSRS |
| | | ADAPAYQQGQNQLYNELNLGRREEYDVLDKRRGRDPEMGGKPRRKNPQ |
| | | EGLYNELQKDKMAEAYSEIGMKGERRRGKGHDGLYQGLSTATKDTYDA |
| | | LHMQALPPR |
| 34 | Myc-IL7 | MLLLVTSLLLCELPHPAFLLIPEQKLISEEDLDCDIEGKDGKQYESVL |
| | degron- | MVSIDQLLDSMKEIGSNCLNNEFNFFKRHICDANKEGMFLFRAARKLR |
| | P2A- | QFLKMNSTGDFDLHLLKVSEGTTILLNCTGQVKGRKPAALGEAQPTKS |
| | CAR19- | LEENKSLKEQKKLNDLCFLKRLLQEIKTCWNKILMGTKEHWNTTKQEH |
| | T2A- | FPDNLLPSWAITLISVNGIFVICCLTYCFAPRCRERRRNERLRRESVR |
| | mCherry | PVGSGSGSGSGGFNVLMVHKRSHTGERPLQCEICGFTCRQKGNLLRHI |
| | | KLHTGEKPFKCHLCNYACQRRDALGSGATNFSLLKQAGDVEENPGPMA |
| | | LPVTALLLPLALLLHAARPEIVMTQSPATLSLSPGERATLSCRASQDI |
| | | SKYLNWYQQKPGQAPRLLIYHTSRLHSGIPARFSGSGSGTDYTLTISS |
| | | LQPEDFAVYFCQQGNTLPYTFGQGTKLEIKGGGGSGGGGSGGGGSGGG |
| | | GSQVQLQESGPGLVKPSETLSLTCTVSGVSLPDYGVSWIRQPPGKGLE |
| | | WIGVIWGSETTYYQSSLKSRVTISKDNSKNQVSLKLSSVTAADTAVYY |
| | | CAKHYYYGGSYAMDYWGQGTLVTVSSTTTPAPRPPTPAPTIASQPLSL |
| | | RPEACRPAAGGAVHTRGLDFACDIYIWAPLAGTCGVLLLSLVITLYCK |
| | | RGRKKLLYIFKQPFMRPVQTTQEEDGCSCRFPEEEEGGCELRVKFSRS |
| | | ADAPAYQQGQNQLYNELNLGRREEYDVLDKRRGRDPEMGGKPRRKNPQ |
| | | EGLYNELQKDKMAEAYSEIGMKGERRRGKGHDGLYQGLSTATKDTYDA |
| | | LHMQALPPRSGGGGEGRGSLLTCGDVEENPGPRMVSKGEEDNMAIIKE |
| | | FMRFKVHMEGSVNGHEFEIEGEGEGRPYEGTQTAKLKVTKGGPLPFAW |
| | | DILSPQFMYGSKAYVKHPADIPDYLKLSFPEGFKWERVMNFEDGGVVT |
| | | VTQDSSLQDGEFIYKVKLRGTNFPSDGPVMQKKTMGWEASSERMYPED |
| | | GALKGEIKQRLKLKDGGHYDAEVKTTYKAKKPVQLPGAYNVNIKLDIT |
| | | SHNEDYTIVEQYERAEGRHSTGGMDELYK |
| 35 | CAR19- | MALPVTALLLPLALLLHAARPEIVMTQSPATLSLSPGERATLSCRASQ |
| | P2A-myc- | DISKYLNWYQQKPGQAPRLLIYHTSRLHSGIPARFSGSGSGTDYTLTI |
| | IL7 | SSLQPEDFAVYFCQQGNTLPYTFGQGTKLEIKGGGGSGGGGSGGGGSG |
| | degron- | GGGSQVQLQESGPGLVKPSETLSLTCTVSGVSLPDYGVSWIRQPPGKG |
| | T2A- | LEWIGVIWGSETTYYQSSLKSRVTISKDNSKNQVSLKLSSVTAADTAV |
| | mCherry | YYCAKHYYYGGSYAMDYWGQGTLVTVSSTTTPAPRPPTPAPTIASQPL |
| | | SLRPEACRPAAGGAVHTRGLDFACDIYIWAPLAGTCGVLLLSLVITLY |
| | | CKRGRKKLLYIFKQPFMRPVQTTQEEDGCSCRFPEEEEGGCELRVKES |
| | | RSADAPAYQQGQNQLYNELNLGRREEYDVLDKRRGRDPEMGGKPRRKN |
| | | PQEGLYNELQKDKMAEAYSEIGMKGERRRGKGHDGLYQGLSTATKDTY |
| | | DALHMQALPPRGSGATNFSLLKQAGDVEENPGPMLLLVTSLLLCELPH |
| | | PAFLLIPEQKLISEEDLDCDIEGKDGKQYESVLMVSIDQLLDSMKEIG |
| | | SNCLNNEFNFFKRHICDANKEGMELFRAARKLRQFLKMNSTGDEDLHL |
| | | LKVSEGTTILLNCTGQVKGRKPAALGEAQPTKSLEENKSLKEQKKLND |
| | | LCFLKRLLQEIKTCWNKILMGTKEHWNTTKQEHFPDNLLPSWAITLIS |
| | | VNGIFVICCLTYCFAPRCRERRRNERLRRESVRPVGSGSGSGSGGENV |
| | | LMVHKRSHTGERPLQCEICGFTCRQKGNLLRHIKLHTGEKPFKCHLCN |
| | | YACQRRDALSGGGGEGRGSLLTCGDVEENPGPRMVSKGEEDNMAIIKE |
| | | FMRFKVHMEGSVNGHEFEIEGEGEGRPYEGTQTAKLKVTKGGPLPFAW |
| | | DILSPQFMYGSKAYVKHPADIPDYLKLSFPEGFKWERVMNFEDGGVVT |
| | | VTQDSSLQDGEFIYKVKLRGTNFPSDGPVMQKKTMGWEASSERMYPED |
| | | GALKGEIKQRLKLKDGGHYDAEVKTTYKAKKPVQLPGAYNVNIKLDIT |
| | | SHNEDYTIVEQYERAEGRHSTGGMDELYK |
| 36 | Mesothelin | MALPVTALLLPLALLLHAARPQVQLQQSGPELEKPGASVKISCKASGY |
| | CAR, P2A, | SFTGYTMNWVKQSHGKSLEWIGLITPYNGASSYNQKFRGKATLTVDKS |
| | myctag- | SSTAYMDLLSLTSEDSAVYFCARGGYDGRGFDYWGQGTTVTVSSGGGG |
| | mbdIL7, | SGGGGSGGGGSDIELTQSPAIMSASPGEKVTMTCSASSSVSYMHWYQQ |
| | T2A, | KSGTSPKRWIYDTSKLASGVPGRFSGSGSGNSYSLTISSVEAEDDATY |
| | mCherry | YCQQWSGYPLTFGAGTKLEITTTPAPRPPTPAPTIASQPLSLRPEACR |
| | | PAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAY |
| | | QQGQNQLYNELNLGRREEYDVLDKRRGRDPEMGGKPRRKNPQEGLYNE |
| | | LQKDKMAEAYSEIGMKGERRRGKGHDGLYQGLSTATKDTYDALHMQAL |
| | | PPRGSGATNFSLLKQAGDVEENPGPMLLLVTSLLLCELPHPAFLLIPE |
| | | QKLISEEDLDCDIEGKDGKQYESVLMVSIDQLLDSMKEIGSNCLNNEF |
| | | NFFKRHICDANKEGMFLFRAARKLRQFLKMNSTGDEDLHLLKVSEGTT |
| | | ILLNCTGQVKGRKPAALGEAQPTKSLEENKSLKEQKKLNDLCFLKRLL |
| | | QEIKTCWNKILMGTKEHWNTTKQEHFPDNLLPSWAITLISVNGIFVIC |
| | | CLTYCFAPRCRERRRNERLRRESVRPVGSGSGSGSGGFNVLMVHKRSH |
| | | TGERPLQCEICGFTCRQKGNLLRHIKLHTGEKPFKCHLCNYACQRRDA |
| | | LSGGGGEGRGSLLTCGDVEENPGPRMVSKGEEDNMAIIKEFMRFKVHM |
| | | EGSVNGHEFEIEGEGEGRPYEGTQTAKLKVTKGGPLPFAWDILSPQFM |
| | | YGSKAYVKHPADIPDYLKLSFPEGFKWERVMNFEDGGVVTVTQDSSLQ |
| | | DGEFIYKVKLRGTNFPSDGPVMQKKTMGWEASSERMYPEDGALKGEIK |
| | | QRLKLKDGGHYDAEVKTTYKAKKPVQLPGAYNVNIKLDITSHNEDYTI |
| | | VEQYERAEGRHSTGGMDELYK |
| 37 | Mesothelin | MALPVTALLLPLALLLHAARPQVQLQQSGPELEKPGASVKISCKASGY |
| | CAR, P2A, | SFTGYTMNWVKQSHGKSLEWIGLITPYNGASSYNQKFRGKATLTVDKS |
| | myctag- | SSTAYMDLLSLTSEDSAVYFCARGGYDGRGFDYWGQGTTVTVSSGGGG |
| | mbdIL18, | SGGGGSGGGGSDIELTQSPAIMSASPGEKVTMTCSASSSVSYMHWYQQ |
| | T2A, | KSGTSPKRWIYDTSKLASGVPGRFSGSGSGNSYSLTISSVEAEDDATY |
| | mCherry | YCQQWSGYPLTFGAGTKLEITTTPAPRPPTPAPTIASQPLSLRPEACR |
| | | PAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAY |
| | | QQGQNQLYNELNLGRREEYDVLDKRRGRDPEMGGKPRRKNPQEGLYNE |
| | | LQKDKMAEAYSEIGMKGERRRGKGHDGLYQGLSTATKDTYDALHMQAL |
| | | PPRGSGATNFSLLKQAGDVEENPGPMLLLVTSLLLCELPHPAFLLIPE |
| | | QKLISEEDLYFGKLESKLSVIRNLNDQVLFIDQGNRPLFEDMTDSDCR |
| | | DNAPRTIFIISMYKDSQPRGMAVTISVKCEKISTLSCENKIISFKEMN |
| | | PPDNIKDTKSDIIFFQRSVPGHDNKMQFESSSYEGYFLACEKERDLFK |
| | | LILKKEDELGDRSIMFTVQNEDWNTTKQEHFPDNLLPSWAITLISVNG |
| | | IFVICCLTYCFAPRCRERRRNERLRRESVRPVGSGSGSGSGGFNVLMV |
| | | HKRSHTGERPLQCEICGFTCRQKGNLLRHIKLHTGEKPFKCHLCNYAC |
| | | QRRDALSGGGGEGRGSLLTCGDVEENPGPRMVSKGEEDNMAIIKEFMR |
| | | FKVHMEGSVNGHEFEIEGEGEGRPYEGTQTAKLKVTKGGPLPFAWDIL |
| | | SPQFMYGSKAYVKHPADIPDYLKLSFPEGFKWERVMNFEDGGVVTVTQ |
| | | DSSLQDGEFIYKVKLRGTNFPSDGPVMQKKTMGWEASSERMYPEDGAL |
| | | KGEIKQRLKLKDGGHYDAEVKTTYKAKKPVQLPGAYNVNIKLDITSHN |
| | | EDYTIVEQYERAEGRHSTGGMDELYK |

### Mammalian Cells

One aspect of the technology described herein relates to a mammalian cell including any T cell factor described herein. In some embodiments, the mammalian cell comprises a CAR polypeptide (e.g., described herein or known in the art). In some embodiments CAR T cells comprising T cell factors of the disclosure have similar in vitro effects to traditional CAR T against models of leukemia and lymphoma, but have distinct transcriptional profiles associated with lower levels of exhaustion. In some embodiments, CAR T cells comprising a T cell factor of the disclosure have enhanced (e.g., increased) anti-tumor activity compared to CAR T cells not comprising a T cell factor. In some embodiments, CAR T cells comprising a T cell factor have increased expansion and/or persistence in the blood, bone marrow, and/or spleen compared to CAR T cells not comprising a T cell factor. Without wishing to be bound by theory, increases in CAR T cell effectiveness and expansion have been associated with CAR T toxic effects in patients that vitiate therapeutic gains. In some embodiments, the inclusion of a degron domain in the T cell factor allows for spatial and temporal control of pro-persistence cytokine signaling, leading to a more effective CAR T treatment. CAR T cells of the disclosure may provide controllable CAR T therapy for patients that are currently not responding to conventional treatments (e.g., due to insufficient persistence or expansion of conventional CAR T therapies).

In some embodiments, the mammalian cell comprises a CAR and a T cell factor comprising a cytokine domain comprising IL-7 or a functional portion thereof, and a degron domain comprising a CRBN polypeptide substrate domain (e.g., comprising IKZF3, a functional portion thereof, or a hybrid fusion polypeptide comprising a functional portion thereof (e.g., and ZFP91 or a functional portion thereof) (and optionally a transmembrane domain comprising a CD80 transmembrane domain). In some embodiments, such exemplary mammalian cells are referred to herein as membrane-bound degradable IL-7 (mbdIL7) CAR T cells.

In some embodiments, the mammalian cell comprises another therapeutic agent (e.g., an antibody reagent (e.g., a scFv, a camelid antibody, or a TEAM) or a cytokine)).

In some embodiments, the mammalian cell comprises a nucleic acid molecule comprising a sequence encoding a T cell factor described herein. In some embodiments, the mammalian cell comprises a nucleic acid molecule comprising a sequence encoding any of the CAR polypeptides described herein or known in the art (optionally together with another therapeutic agent (e.g., an antibody reagent (e.g., a scFv, a camelid antibody, or a TEAM) or a cytokine)). In some embodiments, the mammalian cell includes an antibody, antibody reagent, antigen-binding portion thereof, any of the CARs described herein, or a cytokine, or a nucleic acid encoding such an antibody, antibody reagent, antigen-binding portion thereof, any of the CARs described herein, or a cytokine. The mammalian cell or tissue can be of human, primate, hamster, rabbit, rodent, cow, pig, sheep, horse, goat, dog or cat origin, but any other mammalian cell may be used. In a preferred embodiment of any aspect, the mammalian cell is human.

In some embodiments of any aspect, the mammalian cell is an immune cell. As used herein, “immune cell” refers to a cell that plays a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes. In some embodiments, the immune cell is a T cell; a NK cell; a NKT cell; lymphocytes, such as B cells and T cells; and myeloid cells, such as monocytes, macrophages, eosinophils, mast cells, basophils, and granulocytes. In some embodiments, the immune cell is a T cell.

In some embodiments, the immune cell is obtained from an individual having or diagnosed as having cancer, a plasma cell disorder, or autoimmune disease.

In some embodiments, a mammalian cell, e.g., a T cell, can be engineered to include any of the T cell factors described herein. In some embodiments, a mammalian cell, e.g., a T cell, can be engineered to include any of the CAR polypeptides described herein or known in the art, and optionally a therapeutic agent, as described herein. In some embodiments, a mammalian cell, e.g., a T cell, can be engineered to include a nucleic acid encoding any of the T cell factors, CAR polypeptides, and/or therapeutic agents described herein. T cells can be obtained from a subject using standard techniques known in the field. For example, T cells can be isolated from peripheral blood taken from a donor or patient. T cells can be isolated from a mammal. Preferably, T cells are isolated from a human.

In some embodiments, the disclosure provides constructs that each include separate coding sequences for multiple proteins to be expressed in a mammalian cell (e.g., CAR T cell) of the disclosure. These separate coding sequences can be separated from one another by a cleavable linker sequence as described herein. For example, sequences encoding viral 2A proteins (e.g., T2A and P2A) can be placed between the separate genes and, when transcribed, can direct cleavage of the generated polyprotein. As noted above, constructs and vectors of the disclosure can include any of a number of different combinations of sequences. For example, a construct or vector of the disclosure can include sequences encoding a T cell factor as described herein and a CAR as described herein or known in the art, optionally in combination with a therapeutic agent (e.g., an antibody reagent (e.g., a single chain antibody, a single domain antibody (e.g., a camelid), or a bispecific antibody (e.g., a TEAM)) or a cytokine) as described herein.

Efficient expression of proteins in CAR T cells as described herein can be assessed using standard assays that detect the mRNA, DNA, or gene product of the nucleic acid encoding the proteins. For example, RT-PCR, FAGS, northern blotting, western blotting, ELISA, or immunohistochemistry can be used. The proteins described herein can be constitutively expressed or inducibly expressed. In some embodiments, the proteins are encoded by a recombinant nucleic acid sequence. For example, the disclosure provides a vector that includes a first polynucleotide sequence encoding a T cell factor and a second polynucleotide sequence encoding a CAR, wherein the CAR includes an extracellular domain including an antigen-binding sequence that binds to, e.g., a tumor antigen or a Treg-associated antigen. Optionally, a third polynucleotide sequence encoding a therapeutic agent (e.g., an antibody reagent (e.g., a single chain antibody, a single domain antibody (e.g., a camelid), or a bispecific antibody (e.g., a TEAM)) or a cytokine) may be included in said exemplary vector.

In some embodiments, the first polynucleotide sequence and the second polynucleotide sequence are each operably linked to a promoter, and optionally the third polynucleotide is also operably linked to the promoter. In some embodiments, the first polynucleotide sequence is operably linked to a first promoter and the second polynucleotide sequence is operably linked to a second promoter. In some such embodiments, the third polynucleotide is operably linked to the first promoter, the second promoter, or a third promoter. Promoters can be a constitutively expressed promoter (e.g., an EF1a promoter) or an inducibly expressed promoter (e.g., a NFAT promoter). In some embodiments, a promoter is induced by CAR activity or T cell receptor (TCR) activity.

In some embodiments, expression of the T cell factor and CAR (and optionally the therapeutic agent) are driven by the same promoter, e.g., a constitutively expressed promoter (e.g., an EF1a promoter). In other embodiments, expression of the T cell factor and CAR (and optionally the therapeutic agent) are driven by different promoters. For instance, expression of the CAR can be driven by a constitutively expressed promoter (e.g., an EF1a promoter) while expression of the T cell factor can be driven by an inducibly expressed promoter (e.g., a NFAT promoter). The polynucleotide sequence encoding the CAR can be located upstream of the polynucleotide sequence encoding the T cell factor, or the polynucleotide sequence encoding the T cell factor can be located upstream of the polynucleotide sequence encoding the CAR.

In some embodiments, the polynucleotides can include the expression of a suicide gene.

This can be done to facilitate external, drug-mediated control of administered cells. For example, by use of a suicide gene, modified cells can be depleted from the patient in case of, e.g., an adverse event. In some embodiments, the FK506 binding domain is fused to the caspase9 pro-apoptotic molecule. T cells engineered in this manner are rendered sensitive to the immunosuppressive drug tacrolimus. Other examples of suicide genes are thymidine kinase (TK), CD20, thymidylate kinase, truncated prostate-specific membrane antigen (PSMA), truncated low affinity nerve growth factor receptor (LNGFR), truncated co 19, and modified Fas, which can be triggered for conditional ablation by the administration of specific molecules (e.g., ganciclovir to TK+ ceils) or antibodies or antibody-drug conjugates.

Constructs including sequences encoding proteins for expression in the mammalian cells (e.g., CAR T cells) can be included within vectors. In various examples, the vectors are retroviral vectors. Retroviruses, such as lentiviruses, provide a convenient platform for delivery of nucleic acid sequences encoding a gene, or chimeric gene of interest. A selected nucleic acid sequence can be inserted into a vector and packaged in retroviral particles using techniques known in the art. The recombinant virus can then be isolated and delivered to cells, e.g., in vitro or ex vivo. Retroviral systems are well known in the art and are described in, for example, U.S. Pat. No. 5,219,740; Kurth and Bannert (2010) “Retroviruses: Molecular Biology, Genomics and Pathogenesis” Galster Academic Press (ISBN:978-1-90455-55-4); and Hu and Pathak Pharmacological Reviews 2000 52:493-512; which are incorporated by reference herein in their entirety. Lentiviral system for efficient DNA delivery can be purchased from OriGene; Rockville, MD. In various embodiments, the protein is expressed in the T cell by transfection or electroporation of an expression vector including nucleic acid encoding the protein using vectors and methods that are known in the art. In some embodiments, the vector is a viral vector or a non-viral vector.

In some embodiments, the viral vector is a retroviral vector (e.g., a lentiviral vector), an adenovirus vector, or an adeno-associated virus vector.

### T Cell Factor, CAR, and Therapeutic Agent Expression

In some embodiments of any aspect, any of the T cell factors and/or CAR polypeptides (optionally together with an antibody reagent as described herein or a cytokine) described herein are expressed from one or more lentiviral vectors. The lentiviral vector(s) is used to express the T cell factor and CAR polypeptide (and optionally also the antibody reagent or cytokine) in a cell using infection standard techniques.

Retroviruses, such as lentiviruses, provide a convenient platform for delivery of nucleic acid sequences encoding a gene or chimeric gene of interest. A selected nucleic acid sequence can be inserted into a vector and packaged in retroviral particles using techniques known in the art. The recombinant virus can then be isolated and delivered to cells, e.g., in vitro or ex vivo. Retroviral systems are well known in the art and are described in, for example, U.S. Pat. No. 5,219,740; Kurth and Bannert (2010) “Retroviruses: Molecular Biology, Genomics and Pathogenesis” Galster Academic Press (ISBN:978-1-90455-55-4); and Hu et al., Pharmacological Reviews 52:493-512, 2000; which are each incorporated by reference herein in their entirety. Lentiviral system for efficient DNA delivery can be purchased from OriGene; Rockville, MD. In some embodiments, the T cell factor and/or CAR polypeptide (and optionally the antibody reagent or cytokine) is expressed in a mammalian cell via transfection or electroporation of an expression vector including a nucleic acid encoding the T cell factor and/or CAR.

Transfection or electroporation methods are known in the art.

Efficient expression of the T cell factor and/or CAR polypeptide (and optionally the antibody reagent or cytokine) can be assessed using standard assays that detect the mRNA, DNA, or gene product of the nucleic acid encoding the T cell factor and/or CAR (and optional antibody reagent or cytokine), such as RT-PCR, FAGS, northern blotting, western blotting, ELISA, or immunohistochemistry.

In some embodiments, the T cell factor and/or CAR polypeptide (and optional antibody reagent or cytokine) described herein is constitutively expressed. In other embodiments, one of the T cell factor and CAR polypeptide is constitutively expressed and other is inducibly expressed. In some embodiments, the T cell factor and/or CAR polypeptide (and optional antibody reagent or cytokine) is encoded by a recombinant nucleic acid sequence.

### Methods of Producing a Drug-Responsive T Cell

One aspect of the technology described herein relates to a method of modifying a T cell to produce a drug-responsive T cell, the method including: engineering a T cell to include any of the T cell factors described herein. In some embodiments, the T cell to be modified comprises a CAR polypeptides (and optional antibody reagents or cytokines) described herein or known in the art on the T cell surface. Such methods provide drug-responsive T cells (e.g., drug-responsive CAR T cells) that can, in response to administration of a (e.g., sub-therapeutic) dose of a drug, have their properties (e.g., persistence and/or expansion tendencies, activation state, or phenotypic properties) altered. Many CAR T cell therapies are known in the art, both in active clinical use and in active development. Methods of modifying a T cell to produce a drug-responsive T cell comprising a T cell factor described herein enable the enhancement of any CAR T cell known in the art with drug-responsive properties, thus allowing control of persistence and expansion in diverse CAR T therapies targeting diverse diseases and conditions.

In some embodiments, engineering a T cell to include a T cell factor described herein comprises contacting a T cell (e.g., a CAR T cell) with a nucleic acid molecule described herein (e.g., encoding a T cell factor). Contacting may comprise any suitable method for transferring a nucleic acid known in the art, e.g., transfection, transduction, electroporation, etc. In some embodiments, the nucleic acid molecule is integrated into the genome of the T cell. In some embodiments, the T cell contacted is a CAR T cell. In some embodiments, the T cell contacted is not a CAR T cell. In some embodiments, the T cell is contacted with a polynucleotide encoding a T cell factor and a polynucleotide encoding a CAR (e.g., on the same or separate nucleic acid molecules). Cells receiving nucleic acid molecules can be selected for by methods known in the art, e.g., cell sorting, e.g., by FACS.

In some embodiments, a cell described herein is engineered to express a CRBN polypeptide. In some embodiments, a cell described herein is engineered to heterologously express CRBN, e.g., from an exogenous copy of the CRBN gene. In some embodiments, a mammalian cell described herein comprises a heterologous polynucleotide comprising a sequence encoding CRBN. Without wishing to be bound by theory, the drug-responsiveness of a mammalian cell described herein may be enhanced by an increased level of CRBN, for example by increasing the rapidity with which a T cell factor comprising a CRBN polypeptide substrate domain is bound, ubiquitinated, and/or degraded. In some embodiments, engineering a cell to express a CRBN polypeptide comprises engineering the cell to over-express CRBN. In some embodiments, a heterologous polynucleotide comprising a sequence encoding CRBN comprises a promoter operably linked to CRBN that over-expresses CRBN, e.g., in a T cell.

### Chimeric Antigen Receptors (CARs)

The disclosure is directed, in part, to mammalian cells comprising T cell factors. In some embodiments, a mammalian cell described herein comprises a chimeric antigen receptor (CAR), e.g., in addition to a T cell factor. The disclosure provides CARs for use in immunotherapy. The following discusses CARs and the various improvements and features that may be included in said CARs.

The terms “chimeric antigen receptor” or “CAR” or “CARs”, as used herein, refer to engineered T cell receptors, which graft a ligand or antigen specificity onto T cells (for example, naïve T cells, central memory T cells, effector memory T cells or combinations thereof). CARs are also known as artificial T-cell receptors, chimeric T-cell receptors or chimeric immunoreceptors.

A CAR places a chimeric extracellular antigen-binding domain that specifically binds a target, e.g., a polypeptide, expressed on the surface of a cell to be targeted for a T cell response onto a construct including a transmembrane domain and intracellular domain(s) of a T cell receptor molecule. In some embodiments, the chimeric extracellular antigen-binding domain includes the antigen-binding domain(s) of an antibody reagent that specifically binds an antigen expressed on a cell to be targeted for a T cell response. In some embodiments, the chimeric extracellular antigen-binding domain includes a ligand that specifically binds an antigen expressed on a cell to be targeted for a T cell response.

As used herein, a “CART cell”, “CAR-T cell”, or “CAR T cell” refers to a T cell that expresses a CAR. When expressed in a T cell, CARs have the ability to redirect T-cell specificity and reactivity toward a selected target in a non-MHC-restricted manner, exploiting the antigen-binding properties of monoclonal antibodies. The non-MHC-restricted antigen recognition gives T-cells expressing CARs the ability to recognize an antigen independent of antigen processing, thus bypassing a major mechanism of tumor escape.

In some embodiments, the CAR polypeptide comprises an amino acid sequence with at least 75%, at least 80%, at least 85%, at least 90%, at least 91%, at least 92%, at least 93%, at least 94%, at least 95%, at least 96%, at least 97%, at least 98%, at least 99% or greater sequence identity of a sequence selected from any one of SEQ ID NOs: 54-56. In some embodiments, the CAR polypeptide comprises an amino acid sequence of any one of SEQ ID NOs: 54-56. In some embodiments, the CAR polypeptide consists of an amino acid sequence of any one of SEQ ID NOs: 54-56. In some embodiments, the CAR excludes a CD8 signal peptide as described herein. As can be determined by those of skill in the art, various functionally similar or equivalent components of these CARs can be swapped or substituted with one another, as well as other similar or functionally equivalent components known in the art or listed herein.

### Extracellular Antigen-Binding Domain

As used herein, the term “extracellular antigen-binding domain” refers to a polypeptide found on the outside of the cell that is sufficient to facilitate binding to a target. The extracellular target binding domain will specifically bind to its binding partner, i.e., the target. As non-limiting examples, the extracellular antigen-binding domain can include an antigen-binding domain of an antibody or antibody reagent, or a ligand, which recognizes and binds with a cognate binding partner protein. In this context, a ligand is a molecule that binds specifically to a portion of a protein and/or receptor. The cognate binding partner of a ligand useful in the methods and compositions described herein can generally be found on the surface of a cell. Ligand:cognate partner binding can result in the alteration of the ligand-bearing receptor, or activate a physiological response, for example, the activation of a signaling pathway. In some embodiments, the ligand can be non-native to the genome. Optionally, the ligand has a conserved function across at least two species.

Any cell-surface moiety can be targeted by a CAR. Often, the target will be a cell-surface polypeptide that may be differentially or preferentially expressed on a cell that one wishes to target for a T cell response. Cell surface moieties are further discussed below in “Antibody Reagents”.

### Hinge and Transmembrane Domains

Each CAR as described herein includes a transmembrane domain, e.g., a hinge/transmembrane domain, which joins the extracellular antigen-binding domain to the intracellular signaling domain. The binding domain of the CAR is optionally followed by one or more “hinge domains,” which plays a role in positioning the antigen binding domain away from the effector cell surface to enable proper cell/cell contact, antigen binding and activation. A CAR optionally includes one or more hinge domains between the binding domain and the transmembrane domain™. The hinge domain may be derived either from a natural, synthetic, semi-synthetic, or recombinant source.

The hinge domain can include the amino acid sequence of a naturally occurring immunoglobulin hinge region or an altered immunoglobulin hinge region. Illustrative hinge domains suitable for use in the CARs described herein include the hinge region derived from the extracellular regions of type 1 membrane proteins such as CD8 (e.g., CD8alpha), CD4, CD28, 4-1BB, and CD7, which may be wild-type hinge regions from these molecules or may be altered.

In some embodiments, the hinge region is derived from the hinge region of an immunoglobulin-like protein (e.g., IgA, IgD, IgE, IgG, or IgM), CD28, or CD8. In some embodiments, the hinge domain includes a CD8a hinge region.

As used herein, “transmembrane domain” (TM domain) refers to the portion of the CAR that fuses the extracellular binding portion, optionally via a hinge domain, to the intracellular portion (e.g., the costimulatory domain and intracellular signaling domain) and anchors the CAR to the plasma membrane of the immune effector cell. The transmembrane domain is a generally hydrophobic region of the CAR, which crosses the plasma membrane of a cell. The TM domain can be the transmembrane region or fragment thereof of a transmembrane protein (for example a Type I transmembrane protein or other transmembrane protein), an artificial hydrophobic sequence, or a combination thereof. While specific examples are provided herein and used in the Examples, other transmembrane domains will be apparent to those of skill in the art and can be used in connection with alternate embodiments of the technology. A selected transmembrane region or fragment thereof would preferably not interfere with the intended function of the CAR.

As used in relation to a transmembrane domain of a protein or polypeptide, “fragment thereof” refers to a portion of a transmembrane domain that is sufficient to anchor or attach a protein to a cell surface.

In some embodiments, the transmembrane domain or fragment thereof of the CAR described herein includes a transmembrane domain selected from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), 4-1BBL, GITR, CD40, BAFFR, HVEM (LIGHTR), SLAMF7, NKp80 (KLRFI), CD160, CD19, IL2R beta, IL2R gamma, IL7R a, ITGA1, VLA1, CD49a, ITGA4, IA4, CD49D, ITGA6, VLA-6, CD49f, ITGAD, CD1 Id, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

As used herein, a “hinge/transmembrane domain” refers to a domain including both a hinge domain and a transmembrane domain. For example, a hinge/transmembrane domain can be derived from the hinge/transmembrane domain of CD8, CD28, CD7, or 4-1BB. In some embodiments, the hinge/transmembrane domain of a CAR or fragment thereof is derived from or includes the hinge/transmembrane domain of CD8 (e.g., SEQ ID NO: 47, or variants thereof). CD8 is an antigen preferentially found on the cell surface of cytotoxic T lymphocytes. CD8 mediates cell-cell interactions within the immune system, and acts as a T cell co-receptor. CD8 consists of an alpha (CD8alpha or CD8a) and beta (CD813 or CD8b) chain. CD8a sequences are known for a number of species, e.g., human CD8a, (NCBI Gene ID: 925) polypeptide (e.g., NCBI Ref Seq NP 001139345.1) and mRNA (e.g., NCBI Ref Seq NM_000002.12). CD8 can refer to human CD8, including naturally occurring variants, molecules, and alleles thereof. In some embodiments of any of the aspects, e.g., in veterinary applications, CD8 can refer to the CD8 of, e.g., dog, cat, cow, horse, pig, and the like.

Homologs and/or orthologs of human CD8 are readily identified for such species by one of skill in the art, e.g., using the NCBI ortholog search function or searching available sequence data for a given species for sequence similar to a reference CD8 sequence.

In some embodiments, the hinge and transmembrane sequence corresponds to the amino acid sequence of SEQ ID NO: 1, 47, or 48; or includes a sequence with at least 75%, at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 1, 47, or 48 or an amino acid sequence having ≤1, ≤2, ≤3, ≤4, ≤5, ≤6, ≤7, ≤8, ≤9, or ≤10 substitutions relative to any thereof.

### Co-Stimulatory Domains

Each CAR described herein optionally includes the intracellular domain of one or more co-stimulatory molecule or co-stimulatory domain. As used herein, the term “co-stimulatory domain” refers to an intracellular signaling domain of a co-stimulatory molecule. Co-stimulatory molecules are cell surface molecules other than antigen receptors or Fe receptors that provide a second signal required for efficient activation and function of T lymphocytes upon binding to antigen. The co-stimulatory domain can be, for example, the co-stimulatory domain of 4-1BB, CD27, CD28, or OX40. In one example, a 4-1BB intracellular domain (ICD) can be used (see, e.g., below and SEQ ID NO: 49, or variants thereof). Additional illustrative examples of such co-stimulatory molecules include CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70. In some embodiments, the intracellular domain is the intracellular domain of 4-1 BB. 4-1 BB (CD137; TNFRS9) is an activation induced costimulatory molecule, and is an important regulator of immune responses.

4-1BB is a membrane receptor protein, also known as CD137, which is a member of the tumor necrosis factor (TNF) receptor superfamily. 4-1BB is expressed on activated T lymphocytes. 4-1BB sequences are known for a number of species, e.g., human 4-1 BB, also known as TNFRSF9 (NCBI Gene 25 ID: 3604) and mRNA (NCBI Reference Sequence: NM_001561.5). 4-1BB can refer to human 4-1BB, including naturally occurring variants, molecules, and alleles thereof. In some embodiments of any of the aspects, e.g., in veterinary applications, 4-1BB can refer to the 4-1BB of, e.g., dog, cat, cow, horse, pig, and the like. Homologs and/or orthologs of human 4-1BB are readily identified for such species by one of skill in the art, e.g., using the NCBI ortholog search function or searching available sequence data for a given species for sequence similar to a reference 4-1 BB sequence.

In some embodiments, the intracellular domain is the intracellular domain of a 4-1 BB. In some embodiments, the 4-1 BB intracellular domain corresponds to an amino acid sequence selected from SEQ ID NO: 49; or includes a sequence selected from SEQ ID NO: 49; or includes at least 75%, at least 80%, at least 85%, 35 at least 90%, at least 95%, at least 96%, at least 97%, at least 98%, at least 99%, or at least 100% sequence identity to a sequence selected from SEQ ID NO: 49 or an amino acid sequence having ≤1, ≤2, ≤3, ≤4, ≤5, ≤6, ≤7, ≤8, ≤9, or ≤10 substitutions relative to SEQ ID NO: 49.

### Intracellular Signaling Domains

The properties of the intracellular signaling domain(s) of the CAR can vary as known in the art and as disclosed herein, but the chimeric target/antigen-binding domains(s) render the receptor sensitive to signaling activation when the chimeric target/antigen binding domain binds the target/antigen on the surface of a targeted cell.

With respect to intracellular signaling domains, so-called “first-generation” CARs include those that solely provide CD3-zeta signals upon antigen binding. So-called “second-generation” CARs include those that provide both co-stimulation (e.g., CD28 or CD137) and activation (CD3-zeta;) domains, and so-called “third-generation” CARs include those that provide multiple costimulatory (e.g., CD28 and CD137) domains and activation domains (e.g., CD3-zeta). In various embodiments, the CAR is selected to have high affinity or avidity for the target/antigen—for example, antibody-derived target or antigen binding domains will generally have higher affinity and/or avidity for the target antigen than would a naturally occurring T cell receptor. This property, combined with the high specificity one can select for an antibody provides highly specific T cell targeting by CART cells.

CARs as described herein include an intracellular signaling domain. An “intracellular signaling domain,” refers to the part of a CAR polypeptide that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited following antigen binding to the extracellular CAR domain. In various examples, the intracellular signaling domain is from CD3-zeta; (see, e.g., below). Additional non-limiting examples of immunoreceptor tyrosine-based activation motif (ITAM)-containing intracellular signaling domains that are of particular use in the technology include those derived from TCR-zeta; FcR-gamma, FcR-beta, CD3-gamma, CD3-theta, CD3-sigma, CD3-eta, CD3-epsilon, CD3-zeta; CD22, CD79a, CD79b, and CD66d.

CD3 is a T cell co-receptor that facilitates T lymphocyte activation when simultaneously engaged with the appropriate co-stimulation (e.g., binding of a co-stimulatory molecule). A CD3 complex consists of 4 distinct chains; mammalian CD3 consists of a CD3-gamma chain, a CD3delta chain, and two CD3-epsilon chains.

These chains associate with a molecule known as the T cell receptor (TCR) and the CD3-zeta to generate an activation signal in T lymphocytes. A complete TCR complex includes a TCR, CD3-zeta; and the complete CD3 complex.

In some embodiments of any aspect, a CAR polypeptide described herein includes an

intracellular signaling domain that includes an Immunoreceptor Tyrosine-based Activation Motif or ITAM from CD3-zeta, including variants of CD3-zeta; such as ITAM-mutated CD3-zeta, CD3-eta, or CD3-theta. In some embodiments of any aspect, the ITAM includes three motifs of ITAM of CD3-zeta; (ITAM3). In some embodiments of any aspect, the three motifs of ITAM of CD3-zeta; are not mutated and, therefore, include native or wild-type sequences. In some embodiments, the CD3-zeta; sequence includes the sequence of a CD3-zeta; as set forth in the sequences provided herein, e.g., a CD3-zeta; sequence of SEQ ID NO: 50, or variants thereof.

For example, a CAR polypeptide described herein includes the intracellular signaling domain of CD3-zeta. In some embodiments, the CD3-zeta; intracellular signaling domain corresponds to an amino acid sequence of SEQ ID NO: 50; or includes a sequence selected of SEQ ID NO: 50; or includes a sequence with at least 75%, at least 80%, at least 85%, at least 90%, at least 91%, at least 92%, at least 93%, at least 94%, at least 95%, at least 96%, at least 97%, at least 98%, at least 99%, or at least 100% sequence identity to a sequence of SEQ ID NO: 50 or an amino acid sequence having ≤1, ≤2, ≤3, ≤4, ≤5, ≤6, ≤7, ≤8, ≤9, or ≤10 substitutions relative to SEQ ID NO: 50.

Individual CAR and other construct components as described herein can be used with one another and swapped in and out of various constructs described herein, as can be determined by those of skill in the art. Each of these components can include or consist of any of the corresponding sequences set forth herein, or variants thereof.

A more detailed description of CARs and CART cells can be found in Maus et al., Blood 123:2624-2635, 2014; Reardon et al., Neuro-Oncology 16:1441-1458, 2014; Hoyos et al., Haematologica 97:1622, 2012; Byrd et al., J. Clin. Oncol. 32:3039-3047, 2014; Maher et al., Cancer Res 69:4559-4562, 2009; and Tamada et al., Clin. Cancer Res. 18:6436-6445, 2012; each of which is incorporated by reference herein in its entirety.

### Signal Peptide

In some embodiments, a CAR polypeptide or T cell factor as described herein includes a signal peptide. Signal peptides can be derived from any protein that has an extracellular domain or is secreted. A CAR polypeptide or T cell factor as described herein may include any signal peptides known in the art. In some embodiments, the CAR polypeptide or T cell factor includes a CD8 signal peptide, e.g., a CD8 signal peptide corresponding to the amino acid sequence of SEQ ID NO: 52, or including the amino acid sequence of SEQ ID NO: 52, or including an amino acid sequence having at least 75%, at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 52 or an amino acid sequence having ≤1, ≤2, ≤3, ≤4, ≤5, ≤6, ≤7, ≤8, ≤9, or ≤10 substitutions relative to SEQ ID NO: 52. In some embodiments, the CAR polypeptide or T cell factor includes a IgK signal peptide, e.g., a IgK signal peptide corresponding to the amino acid sequence of SEQ ID NO: 53, or including the amino acid sequence of SEQ ID NO: 53, or including an amino acid sequence having at least 75%, at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 53 or an amino acid sequence having ≤1, ≤2, ≤3, ≤4, ≤5, ≤6, ≤7, ≤8, ≤9, or ≤10 substitutions relative to SEQ ID NO: 53.

In further embodiments, a CAR polypeptide described herein may optionally exclude one of the signal peptides described herein, e.g., a CD8 signal peptide of SEQ ID NO: 52 or an IgK signal peptide of SEQ ID NO: 53.

### Linker Domain

In some embodiments, a CAR or T cell factor further includes a linker domain. As used herein, “linker domain” refers to an oligo- or polypeptide region from about 2 to 100 amino acids in length, which links together any of the domains/regions of the CAR or T cell factor as described herein. In some embodiment, linkers can include or be composed of flexible residues such as glycine and serine so that the adjacent protein domains are free to move relative to one another. Linker sequences useful for the invention can be from 2 to 100 amino acids, 5 to 50 amino acids, 10 to 15 amino acids, 15 to 20 amino acids, or 18 to 20 amino acids in length, and include any suitable linkers known in the art. For instance, linker sequences useful for the invention include, but are not limited to, glycine/serine linkers, e.g., GGGSGGGSGGGS (SEQ ID NO: 38) and Gly4Ser (G4S) linkers such as (G4S)3 (GGGGSGGGGSGGGGS (SEQ ID NO: 39)) and (G4S)4

(GGGGSGGGGSGGGGSGGGGS (SEQ ID NO: 40)); the linker sequence of GSTSGSGKPGSGEGSTKG (SEQ ID NO: 41) as described by Whitlow et al., Protein Eng. 6(8):989-95, 1993, the contents of which are incorporated herein by reference in its entirety; the linker sequence of GGSSRSSSSGGGGSGGGG (SEQ ID NO: 42) as described by Andris-Widhopf et al., Cold Spring Harb. Protoc. 2011 (9), 2011, the contents of which are incorporated herein by reference in its entirety; as well as linker sequences with added functionalities, e.g., an epitope tag or an encoding sequence containing Cre-Lox recombination site as described by Sblattero et al., Nat. Biotechnol. 18(1):75-80, 2000, the contents of which are incorporated herein by reference in its entirety. Longer linkers may be used when it is desirable to ensure that two adjacent domains do not sterically interfere with one another.

Furthermore, linkers may be cleavable or non-cleavable. Examples of cleavable linkers include 2A linkers (e.g., P2A (SEQ ID NO: 43) and T2A (SEQ ID NO: 44), 2A-like linkers or functional equivalents thereof and combinations thereof.

For example, a P2A linker sequence can correspond to the amino acid sequence of SEQ ID NO: 43. In various examples, linkers having sequences as set forth herein, or variants thereof, are used. It is to be understood that the indication of a particular linker in a construct in a particular location does not mean that only that linker can be used there. Rather, different linker sequences (e.g., P2A and T2A) can be swapped with one another (e.g., in the context of the constructs of the present invention), as can be determined by those of skill in the art. In some embodiments, the linker region is T2A derived from Thosea asigna virus. Non-limiting examples of linkers that can be used in this technology include T2A, P2A, E2A, BmCPV2A, and BmlFV2A. Linkers such as these can be used in the context of polyproteins, such as those described below. For example, they can be used to separate a CAR component of a polyprotein from a T cell factor, or a T cell factor or CAR component of a polyprotein from a therapeutic agent (e.g., an antibody, such as a scFv, single domain antibody (e.g., a camelid antibody), or a bispecific antibody (e.g., a TEAM)) component of a polyprotein (see below).

### Reporter Molecule

In some embodiments, a CAR or T cell factor as described herein optionally further includes a reporter molecule, e.g., to permit for non-invasive imaging (e.g., positron-emission tomography PET scan). In a bispecific CAR that includes a reporter molecule, the first extracellular binding domain and the second extracellular binding domain can include different or the same reporter molecule. In a bispecific CART cell, the first CAR and the second CAR can express different or the same reporter molecule. In another embodiment, a CAR as described herein further includes a reporter molecule (for example hygromycin phosphotransferase (hph)) that can be imaged alone or in combination with a substrate or chemical (for example 9-[4-[18F]fluoro-3-(hydroxymethyl)butyl]guanine ([18F]FHBG)). In another embodiment, a CAR as described herein further includes nanoparticles at can be readily imaged using non-invasive techniques (e.g., gold nanoparticles (GNP) functionalized with 64Cu2+). Labeling of CART cells for non-invasive imaging is reviewed, for example in Bhatnagar et al., Integr. Biol. (Camb). 5(1):231-238, 2013, and Keu et al., Sci. Transl. Med. 18; 9(373), 2017, which are incorporated herein by reference in their entireties.

In some embodiments, GFP and mCherry may be used as fluorescent tags for imaging a CAR expressed on a T cell (e.g., a CART cell). It is expected that essentially any fluorescent protein known in the art can be used as a fluorescent tag for this purpose. For clinical applications, the CAR need not include a fluorescent tag or fluorescent protein. In each instance of particular constructs provided herein, therefore, any markers present in the constructs can be removed. The invention includes the constructs with or without the markers. Accordingly, when a specific construct is referenced herein, it can be considered with or without any markers or tags (including, e.g., histidine tags, such as the histidine tag of HHHHHH (SEQ ID NO: 45)) as being included within the invention.

TABLE 4
Exemplary CAR sequences

| SEQ | | |
|---|---|---|
| ID | | |
| NO: | Description | Sequence |
| 46 | Exemplary | EIVMTQSPATLSLSPGERATLSCRASQDISKYLNWYQQKPGQAPRLLI |
| | Anti-CD19 | YHTSRLHSGIPARFSGSGSGTDYTLTISSLQPEDFAVYFCQQGNTLPY |
| | scFv | TFGQGTKLEIKGGGGSGGGGSGGGGSGGGGSQVQLQESGPGLVKPSET |
| | | LSLTCTVSGVSLPDYGVSWIRQPPGKGLEWIGVIWGSETTYYQSSLKS |
| | | RVTISKDNSKNQVSLKLSSVTAADTAVYYCAKHYYYGGSYAMDYWGQG |
| | | TLVTVSS |
| 47 | CD8 | TTTPAPRPPTPAPTIASQPLSLRPEACRPAAGGAVHTRGLDFACDIYI |
| | hinge/TM | WAPLAGTCGVLLLSLVITLYC |
| | domain | |
| 48 | CD28 | TTTPAPRPPTPAPTIASQPLSLRPEACRPAAGGAVHTRGLDFACDIYI |
| | hinge/TM | WAPLAGTCGVLLLSLVITLYC |
| | domain | |
| 49 | 4-1 BB ICD | KRGRKKLLYIFKQPFMRPVQTTQEEDGCSCRFPEEEEGGCEL |
| 50 | CD3-zeta | RVKFSRSADAPAYQQGQNQLYNELNLGRREEYDVLDKRRGRDPEMGGK |
| | ICD | PRRKNPQEGLYNELQKDKMAEAYSEIGMKGERRRGKGHDGLYQGLSTA |
| | | TKDTYDALHMQALPPR |
| 51 | CD28 ICD | RSKRSRLLHSDYMNMTPRRPGPTRKHYQPYAPPRDFAAYRS |
| 52 | CD8 leader | MALPVTALLLPLALLLHAARP |
| 53 | IgK signal | METDTLLLWVLLLWVPGSTGD |
| | sequence | |
| 54 | Exemplary | EIVMTQSPATLSLSPGERATLSCRASQDISKYLNWYQQKPGQAPRLLI |
| | CD19 CAR | YHTSRLHSGIPARFSGSGSGTDYTLTISSLQPEDFAVYFCQQGNTLPY |
| | 1 | TFGQGTKLEIKGGGGSGGGGSGGGGSGGGGSQVQLQESGPGLVKPSET |
| | | LSLTCTVSGVSLPDYGVSWIRQPPGKGLEWIGVIWGSETTYYQSSLKS |
| | | RVTISKDNSKNQVSLKLSSVTAADTAVYYCAKHYYYGGSYAMDYWGQG |
| | | TLVTVSSTTTPAPRPPTPAPTIASQPLSLRPEACRPAAGGAVHTRGLD |
| | | FACDIYIWAPLAGTCGVLLLSLVITLYCKRGRKKLLYIFKQPFMRPVQ |
| | | TTQEEDGCSCRFPEEEEGGCELRVKESRSADAPAYQQGQNQLYNELNL |
| | | GRREEYDVLDKRRGRDPEMGGKPRRKNPQEGLYNELQKDKMAEAYSEI |
| | | GMKGERRRGKGHDGLYQGLSTATKDTYDALHMQALPPR |
| 55 | Exemplary | MALPVTALLLPLALLLHAARPEIVMTQSPATLSLSPGERATLSCRASQ |
| | CD19 CAR | DISKYLNWYQQKPGQAPRLLIYHTSRLHSGIPARFSGSGSGTDYTLTI |
| | 2 | SSLQPEDFAVYFCQQGNTLPYTFGQGTKLEIKGGGGSGGGGSGGGGSG |
| | | GGGSQVQLQESGPGLVKPSETLSLTCTVSGVSLPDYGVSWIRQPPGKG |
| | | LEWIGVIWGSETTYYQSSLKSRVTISKDNSKNQVSLKLSSVTAADTAV |
| | | YYCAKHYYYGGSYAMDYWGQGTLVTVSSTTTPAPRPPTPAPTIASQPL |
| | | SLRPEACRPAAGGAVHTRGLDFACDIYIWAPLAGTCGVLLLSLVITLY |
| | | CKRGRKKLLYIFKQPFMRPVQTTQEEDGCSCRFPEEEEGGCELRVKES |
| | | RSADAPAYQQGQNQLYNELNLGRREEYDVLDKRRGRDPEMGGKPRRKN |
| | | PQEGLYNELQKDKMAEAYSEIGMKGERRRGKGHDGLYQGLSTATKDTY |
| | | DALHMQALPPR |
| 56 | Exemplary | MALPVTALLLPLALLLHAARPQVQLQQSGPELEKPGASVKISCKASGY |
| | mesothelin | SFTGYTMNWVKQSHGKSLEWIGLITPYNGASSYNQKFRGKATLTVDKS |
| | CAR | SSTAYMDLLSLTSEDSAVYFCARGGYDGRGFDYWGQGTTVTVSSGGGG |
| | | SGGGGSGGGGSDIELTQSPAIMSASPGEKVTMTCSASSSVSYMHWYQQ |
| | | KSGTSPKRWIYDTSKLASGVPGRFSGSGSGNSYSLTISSVEAEDDATY |
| | | YCQQWSGYPLTFGAGTKLEITTTPAPRPPTPAPTIASQPLSLRPEACR |
| | | PAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAY |
| | | QQGQNQLYNELNLGRREEYDVLDKRRGRDPEMGGKPRRKNPQEGLYNE |
| | | LQKDKMAEAYSEIGMKGERRRGKGHDGLYQGLSTATKDTYDALHMQAL |
| | | PPR |
| 43 | P2A self- | GSGATNFSLLKQAGDVEENPGP |
| | cleaving | |
| | sequence | |
| 44 | T2A self- | SGGGGEGRGSLLTCGDVEENPGPR |
| | cleaving | |
| | sequence | |
| 57 | mCherry | MVSKGEEDNMAIIKEFMRFKVHMEGSVNGHEFEIEGEGEGRPYEGTQT |
| | | AKLKVTKGGPLPFAWDILSPQFMYGSKAYVKHPADIPDYLKLSFPEGF |
| | | KWERVMNFEDGGVVTVTQDSSLQDGEFIYKVKLRGTNFPSDGPVMQKK |
| | | TMGWEASSERMYPEDGALKGEIKQRLKLKDGGHYDAEVKTTYKAKKPV |
| | | QLPGAYNVNIKLDITSHNEDYTIVEQYERAEGRHSTGGMDELYK |

### Antibody Reagents

In various embodiments, the CARs described herein include an antibody reagent or an antigen-binding domain thereof as an extracellular target-binding domain.

As used herein, the term “antibody reagent” refers to a polypeptide that includes at least one immunoglobulin variable domain or immunoglobulin variable domain sequence and which specifically binds a given antigen. In some embodiments, an antibody reagent can include an antibody or a polypeptide including an antigen-binding domain of an antibody. In some embodiments of any of the aspects, an antibody reagent can include a monoclonal antibody or a polypeptide including an antigen-binding domain of a monoclonal antibody. For example, an antibody can include a heavy (H) chain variable region (abbreviated herein as VH), and a light (L) chain variable region (abbreviated herein as VL). In some embodiments, an antibody includes two heavy (H) chain variable regions and two light (L) chain variable regions. In some embodiments, the antibody reagent is a bispecific antibody reagent.

The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g., de Wildt et al., Eur. J. Immunol. 26(3):629-639, 1996; which is incorporated by reference herein in its entirety)) as well as complete antibodies. An antibody can have the structural features of lgA, lgG, lgE, lgD, or lgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like. In some embodiments, the CAR comprises an antibody reagent. In some embodiments, the therapeutic agent comprises an antibody reagent.

Fully human antibody binding domains can be selected, for example, from phage display libraries using methods known to those of ordinary skill in the art. Furthermore, antibody reagents include single domain antibodies, such as camelid antibodies.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A. et al. (1991) Sequences of Proteins of Immunological Interest, Fifth Edition, U.S. Department of Health and Human Services, NIH Publication No. 91-3242, and Chothia et al., J. Mol. Biol. 196:901-917, 1987; each of which is incorporated by reference herein in its entirety). Each VH and VL is typically composed of three CDRs and four FRs, arranged from amino-terminus to carboxy-terminus in the following order: FR1, CDR1, FR2, CDR2, FR3, CDR3, FR4.

In some embodiments, the antibody or antibody reagent is not a human antibody or antibody reagent (i.e., the antibody or antibody reagent is mouse), but has been humanized. A “humanized antibody or antibody reagent” refers to a non-human antibody or antibody reagent that has been modified at the protein sequence level to increase its similarity to antibody or antibody reagent variants produced naturally in humans. One approach to humanizing antibodies employs the grafting of murine or other non-human CDRs onto human antibody frameworks.

In some embodiments, the extracellular target binding domain of a CAR includes or consists essentially of a single-chain Fv (scFv) fragment created by fusing the VH and VL domains of an antibody, generally a monoclonal antibody, via a flexible linker peptide. In various embodiments, the scFv is fused to a transmembrane domain and to a T cell receptor intracellular signaling domain, e.g., an engineered intracellular signaling domain as described herein. In another embodiment, the extracellular target binding domain of a CAR includes a camelid antibody.

In some embodiments, the antibody reagent binds to a tumor associated-antigen. Non-limiting examples of additional tumor antigens, tumor-associated antigens, or other antigen of interest include activated fibroblast marker, CD19, CD37, BCMA (tumor necrosis factor receptor superfamily member 17 (TNFRSF17); NCBI Gene ID: 608; NCBI Ref Seq: NP 001183.2 and mRNA (e.g., NCBI Ref Seq: NM_001192.2)), CEA, immature laminin receptor, TAG-72, HPV E6 and E7, BING-4, calcium-activated chloride channel 2, cyclin B1, 9D7, Ep-CAM, EphA3, 15 her2/neu, telomerase, EGFR, EGFRviii SAP-1, Survivin, BAGE family, CAGE family, GAGE family, MAGE family, SAGE family, XAGE family, NY-ESO-1/LAGE-1, PRAME, SSX-2, Melan-NMART-1, gp100/pmel17, tyrosinase, TRP-1/-2, MC1R, BRCA1/2, CDK4, MART-2, p53, Ras, MUC1, TGF-BetaRII, IL-15, IL-13Ra2, and CSF1 R. In some embodiments, the activated fibroblast marker comprises any one of αSMA (ACTA2), fibroblast activation protein (FAP), platelet derived growth factor receptor-α and -β (PDGFRA, PDGFRB), fibroblast specific protein 1 (FSP1/S100A4), endoglin (ENG), transgelin (TAGLN), tenascin C (TNC), periostin (POSTN), chondroitin sulphate proteoglycan 4 or neuron-glial antigen 2 (CSPG4/NG2), podoplanin (PDPN), or osteopontin (SPP1).

### Administration and Treatment

### Subject

As used herein, a “subject” means a human or animal. Usually the animal is a vertebrate such as a primate, rodent, domestic animal or game animal. Primates include, for example, chimpanzees, cynomolgus monkeys, spider monkeys, and macaques, e.g., rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient,” and “subject” are used interchangeably herein. Preferably, the subject is a mammal. The mammal can be a human, non-human primate, mouse, rat, dog, cat, horse, or cow, but is not limited to these examples. Mammals other than humans can be advantageously used as subjects that represent animal models of disease, e.g., cancer. A subject can be male or female.

A subject can be one who has been previously diagnosed with or identified as suffering from or having a condition in need of treatment (e.g., a pancreatic cancer, a lung cancer, an ovarian cancer, endometrial cancer, biliary cancer, gastric cancer, or mesothelioma or another type of cancer expressing mesothelin, among others) or one or more complications related to such a condition, and optionally, have already undergone treatment for the condition or the one or more complications related to the condition.

Alternatively, a subject can also be one who has not been previously diagnosed as having such condition or related complications. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

### Pharmaceutical Compositions

As used herein, the term “pharmaceutical composition” refers to the active agent in combination with a pharmaceutically acceptable carrier e.g. a carrier commonly used in the pharmaceutical industry.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier in which the active ingredient would not be found to occur in nature.

In one aspect of the technology, the technology described herein relates to a pharmaceutical composition including activated CART cells comprising a T cell factor as described herein, and optionally a pharmaceutically acceptable carrier. The active ingredients of the pharmaceutical composition at a minimum include activated CART cells comprising a T cell factor as described herein. In some embodiments, the active ingredients of the pharmaceutical composition consist essentially of activated CART cells comprising a T cell factor as described herein. In some embodiments, the active ingredients of the pharmaceutical composition consist of activated CAR T cells comprising a T cell factor as described herein. Pharmaceutically acceptable carriers for cell-based therapeutic formulation include saline and aqueous buffer solutions, Ringer's solution, and serum component, such as serum albumin, HDL and LDL. The terms such as “excipient,” “carrier,” “pharmaceutically acceptable carrier”, “pharmaceutically acceptable excipient” or the like are used interchangeably herein.

In some embodiments, the pharmaceutical composition including activated CAR T cells comprising a T cell factor as described herein can be a parenteral dose form. Since administration of parenteral dosage forms typically bypasses the patient's natural defenses against contaminants, the components apart from the CART cells themselves are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. Any of these can be added to the activated CART cells preparation prior to administration. Suitable vehicles that can be used to provide parenteral dosage forms of activated CAR T cells as disclosed within are well known to those skilled in the art. Examples include, without limitation: saline solution; glucose solution; aqueous vehicles including but not limited to, sodium chloride injection, Ringer's injection, dextrose injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

### Dosage

“Unit dosage form” as the term is used herein refers to a dosage for suitable one administration. By way of example, a unit dosage form can be an amount of therapeutic disposed in a delivery device, e.g., a syringe or intravenous drip bag. In some embodiments, a unit dosage form is administered in a single administration. In another, embodiment more than one unit dosage form can be administered simultaneously.

In some embodiments, the activated CAR T cells comprising a T cell factor described herein are administered as a monotherapy, i.e., another treatment for the condition is not concurrently administered to the subject. A pharmaceutical composition including the T cells described herein can generally be administered at a dosage of 10⁴ to 10⁹ cells/kg body weight, in some instances 10⁵ to 10⁶ cells/kg body weight, including all integer values within those ranges. If necessary, T cell compositions can also be administered multiple times at these dosages. The cells can be administered by using infusion techniques that are commonly known in immunotherapy (see, e.g., Rosenberg et al., New Eng. J. Med. 30 319:1676, 1988).

In certain aspects, it may be desired to administer activated CART cells comprising a T cell factor to a subject and then subsequently redraw blood (or have an apheresis performed), activate T cells therefrom as described herein, and reinfuse the patient with these activated and expanded T cells. This process can be carried out multiple times every few weeks. In certain aspects, T cells can be activated from blood draws of from 35 10 cc to 400 cc. In certain aspects, T cells are activated from blood draws of 20 cc, 30 cc, 40 cc, 50 cc, 60 cc, 70 cc, 80 cc, 90 cc, or 100 cc.

In certain aspects, it may be desired to administer a drug to a subject who has been administered CAR T cells comprising a T cell factor described herein, wherein the drug is recognized by the T cell factor and causes the T cell factor to be degraded.

### Administration

In some embodiments, the methods described herein relate to treating a subject having or diagnosed as having cancer, a plasma cell disease or disorder, or an autoimmune disease or disorder with a mammalian cell including any of the CAR polypeptides (and optional antibody reagents or cytokines) described herein, or a nucleic acid encoding any of the CAR polypeptides (and optional antibody reagents or cytokines) described herein. The CART cells comprising a T cell factor described herein include mammalian cells including any of the T cell factors described herein and any of the CAR polypeptides (and optional antibody reagents or cytokines) described herein or known in the art, or a nucleic acid encoding any of the T cell factors or CAR polypeptides (and optional antibody reagents or cytokines) described herein. Subjects having a condition can be identified by a physician using current methods of diagnosing the condition. Symptoms and/or complications of the condition, which characterize these conditions and aid in diagnosis are well known in the art and include but are not limited to, fatigue, persistent infections, and persistent bleeding. Tests that may aid in a diagnosis of, e.g., the condition, but are not limited to, blood screening and bone marrow testing, and are known in the art for a given condition. A family history for a condition, or exposure to risk factors for a condition can also aid in determining if a subject is likely to have the condition or in making a diagnosis of the condition.

The compositions described herein can be administered to a subject having or diagnosed as having a condition. In some embodiments, the methods described herein include administering an effective amount of activated CAR T cells comprising a T cell factor described herein to a subject in order to alleviate a symptom of the condition. As used herein, “alleviating a symptom of the condition” is ameliorating any condition or symptom associated with the condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. In some embodiments, the compositions described herein are administered systemically or locally. In a preferred embodiment, the compositions described herein are administered intravenously. In another embodiment, the compositions described herein are administered at the site of a tumor.

The term “effective amount” as used herein refers to the amount of activated CAR T cells comprising a T cell factor described herein needed to alleviate at least one or more symptom of the disease or disorder, and relates to a sufficient amount of the cell preparation or composition to provide the desired effect. The term “therapeutically effective amount” therefore refers to an amount of activated CART cells comprising a T cell factor described herein that is sufficient to provide a particular anti-condition effect when administered to a typical subject. An effective amount as used herein, in various contexts, would also include an amount sufficient to delay the development of a symptom of the disease, alter the course of a symptom disease (for example but not limited to, slowing the progression of a condition), or reverse a symptom of the condition. Thus, it is not generally practicable to specify an exact “effective amount.” However, for any given case, an appropriate “effective amount” can be determined by one of ordinary skill in the art using only routine experimentation.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of activated CART cells comprising a T cell factor described herein, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for bone marrow testing, among others. The dosage can be determined by a physician and adjusted, as necessary, to suit observed effects of the treatment.

### Modes of Administration

Modes of administration can include, for example intravenous (iv) injection or infusion. The compositions described herein can be administered to a patient transarterially, intratumorally, intranodally, intraperitoneally or intramedullary. In some embodiments, the compositions of T cells may be injected directly into a tumor, lymph node, or site of infection. In some embodiments, the compositions described herein are administered into a body cavity or body fluid (e.g., ascites, pleural fluid, peritoneal fluid, or cerebrospinal fluid).

In a particular exemplary aspect, subjects may undergo leukapheresis, wherein leukocytes are collected, enriched, or depleted ex vivo to select and/or isolate the cells of interest, e.g., T cells. These T cell isolates can be expanded by contact with an artificial APC, e.g., an aAPC expressing anti-CD28 and anti-CD3 CD Rs, and treated such that one or more CAR constructs of the technology may be introduced, thereby creating a CAR T cell. Subjects in need thereof can subsequently undergo standard treatment with high dose chemotherapy followed by peripheral blood stem cell transplantation. Following or concurrent with the transplant, subjects can receive an infusion of the expanded CAR T cells. In some embodiment, expanded cells are administered before or following surgery. In some embodiments, lymphodepletion is performed on a subject prior to administering one or more CART cell as described herein. In such embodiments, the lymphodepletion can include administering one or more of melphalan, cytoxan, cyclophosphamide, and fludarabine. The dosage of the above treatments to be administered to a patient will vary with the precise nature of the condition being treated and the recipient of the treatment. The scaling of dosages for human administration can be performed according to art-accepted practices.

In some embodiments, a single treatment regimen is required. In others, administration of one or more subsequent doses or treatment regimens can be performed. For example, after treatment biweekly for three months, treatment can be repeated once per month, for six months or a year or longer. In some embodiments, no additional treatments are administered following the initial treatment.

The dosage of a composition as described herein can be determined by a physician and

adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

### Combination Therapy

The activated CART cells comprising a T cell factor described herein can optionally be used in combination with each other and with other known agents and therapies, as can determined to be appropriate by those of skill in the art. In one example, two or more CART cells targeting different Treg markers (e.g., GARP, LAP, CD25, and cytotoxic T lymphocyte-associated antigen-4 (CTLA-4) etc.) can be administered in combination. In another example, two or more CART cells targeting different cancer antigens are administered in combination. In a further example, one or more CART cell targeting a Treg marker (e.g., GARP, LAP, etc.) and one or more CART cell targeting one or more tumor antigens are administered in combination.

Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The activated CART cells comprising a T cell factor described herein and the at least one additional therapeutic agent can be administered simultaneously, in the same or in separate compositions, or sequentially. For sequential administration, the CAR expressing cell described herein can be administered first, and the additional agent can be administered second, or the order of administration can be reversed. The CAR T therapy and/or other therapeutic agents, procedures or modalities can be administered during periods of active disorder, or during a period of remission or less active disease. The CART therapy can be administered before another treatment, concurrently with the treatment, post-treatment, or during remission of the disorder.

When administered in combination, the activated CART cells comprising a T cell factor described herein and the additional agent (e.g., second or third agent), or all, can be administered in an amount or dose that is higher, lower or the same as the amount or dosage of each agent used individually, e.g., as a monotherapy. In certain embodiments, the administered amount or dosage of the activated CART cells, the additional agent (e.g., second or third agent), or all, is lower (e.g., at least 20%, at least 30%, at least 40%, or at least 50%) than the amount or dosage of each agent used individually. In other embodiments, the amount or dosage of the activated CART cells, the additional agent (e.g., second or third agent), or all, that results in a desired effect (e.g., treatment of cancer) is lower (e.g., at least 20%, at least 30%, at least 40%, or at least 50% lower) than the amount or dosage of each agent individually required to achieve the same therapeutic effect. In further embodiments, the activated CART cells described herein can be used in a treatment regimen in combination with surgery, chemotherapy, radiation, an mTOR pathway inhibitor, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, rapamycin, mycophenolic acid, steroids, FR901228, cytokines, or a peptide vaccine, such as that described in Izumoto et al., J. Neurosurg. 108:963-971, 2008.

In some embodiments, the activated CART cells comprising a T cell factor described herein can be used in combination with a checkpoint inhibitor. Exemplary checkpoint inhibitors include anti-PD-1 inhibitors (Nivolumab, MK-3475, Pembrolizumab, Pidilizumab, AMP-224, AMP-514), anti-CTLA4 inhibitors (Ipilimumab and Tremelimumab), anti-POL 1 inhibitors (Atezolizumab, Avelomab, MSB0010718C, MED14736, and MPDL3280A), and anti-TIM3 inhibitors.

In some embodiments, the activated CART cells described herein can be used in combination with a chemotherapeutic agent. Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide). General chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®). Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU@), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AO); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®). Exemplary mTOR inhibitors include, e.g., temsirolimus; ridaforolimus (formally known as deferolimus, (IR,2R,45)-4-[(2R)-2 [(1R,95, 125, 15R, 16E, 18R, 19R,21 R,235,24E,26E,28Z,305,325,35R)-I, 18-dihydroxy-19,30-dimethoxy-15, 17,21,23, 29,35-hexamethyl-2,3, 10, 14,20-pentaoxo-11,36-dioxa-4-azatricyclo[30.3.1.04′9]hexatriaconta-16,24,26,28-tetraen-12-yl]propyl]-2-methoxycyclohexyl dimethylphosphinate, also known AP23573 and MK8669, and described in PCT Publication No. WO 03/064383); everolimus (Afinitor® or RADOOI); rapamycin (AY22989, Sirolimus®); simapimod (CAS 164301-51-3); emsirolimus, (5-{2,4-Bis[(35,)-3-methylmorpholin-4-yl]pyrido[2,3-(i]pyrimidin-7-yl}-2-methoxyphenyl)methanol (AZD8055); 2-Amino-8-[iraw5, -4-(2-hydroxyethoxy)cyclohexyl]-6-(6-methoxy-3-pyridinyl)-4-methyl-pyrido[2,3-Jjpyrimidin-7(8H)-one (PF04691502, CAS 1013101-36-4); and N2-[1,4-dioxo-4-[[4-(4-oxo-8-phenyl-4H-lbenzopyran-2-yl)morpholin ium-4-yl]methoxy]butyl]-L-arginylglycyl-L-a-aspartyl L-serine-, inner salt (SF1126, CAS 936487-67-1), and XL765. Exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon y, CAS 951209-71-5, available from IRX Therapeutics). Exemplary anthracyclines include, e.g., doxorubicin (Adriamycin® and Rubex®); bleomycin (lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (ldamycin®, ldamycin PFS®); mitomycin C (Mutamycin®); geldanamycin; herbimycin; ravidomycin; and desacetylravidomycin. Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®). Exemplary proteosome inhibitors include bortezomib (Velcade®); carfilzomib (PX-171-007, (5)-4-Methyl-N-((5)-1-(((5)-4-methyl-1-(I-2-methyloxiran-2-yl)-1-oxopentan-2-yl)amino)-1-oxo-3-phenylpropan-2-yl)-2-((5,)-2-(2-morphol inoacetamido)-4-phenylbutanamido)-pentanamide); marizom ib(NPT0052); ixazomib citrate (MLN-9708); delanzomib (CEP-18770); and O-Methyl-N-[(2-methyl-5-thiazolyl)carbonyl]-L-seryl-O-methyl-N-[(I IS′)-2-[(2 R)-2-methyl-2-oxi ranyl]-2-oxo-1-(phenyl methyl) ethyl]-L-serinamide (ONX-0912).

One of skill in the art can readily identify a chemotherapeutic agent of use (e.g., see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18^{th} edition; Therapeutic Targeting of Cancer Cells: Era of Molecularly Targeted Agents and Cancer Pharmacology, Chapters 28-29 in Abeloff's Clinical Oncology, 2013 Elsevier; and Fischer D. S. (ed.): The Cancer Chemotherapy Handbook, 4^{th} ed. St. Louis, Mosby-Year Book, 2003).

In an embodiment, activated CAR T cells comprising a T cell factor described herein are administered to a subject in combination with a molecule that decreases the level and/or activity of a molecule targeting GITR and/or modulating GITR functions, a molecule that decreases the Treg cell population, an mTOR inhibitor, a GITR agonist, a kinase inhibitor, a non-receptor tyrosine kinase inhibitor, a CDK4 inhibitor, and/or a BTK inhibitor.

### Efficacy

The efficacy of activated CART cells comprising a T cell factor described herein in, e.g., the treatment of a condition described herein, or to induce a response as described herein (e.g., a reduction in cancer cells) can be determined by the skilled clinician. However, a treatment is considered “effective treatment,” as the term is used herein, if one or more of the signs or symptoms of a condition described herein is altered in a beneficial manner, other clinically accepted symptoms are improved, or even ameliorated, or a desired response is induced, e.g., by at least 10% following treatment according to the methods described herein. Efficacy can be assessed, for example, by measuring a marker, indicator, symptom, and/or the incidence of a condition treated according to the methods described herein or any other measurable parameter appropriate.

Treatment according to the methods described herein can reduce levels of a marker or symptom of a condition, e.g. by at least 10%, at least 15%, at least 20%, at least 25%, at least 30%, at least 40%, at least 50%, at least 60%, at least 70%, at least 80% or at least 90% or more.

Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g., pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy of a given approach can be assessed in animal models of a condition described herein. When using an experimental animal model, efficacy of treatment is evidenced when a statistically significant change in a marker is observed.

### CAR T Cell Therapy

One aspect of the technology described herein relates to a method of treating cancer, a plasma cell disorder, or an autoimmune disease in a subject in need thereof, the method including: engineering a T cell to include any of the T cell factors described herein and include any CAR polypeptides (and optional antibody reagents or cytokines) described herein or known in the art on the T cell surface; and administering the engineered T cell to the subject. In the case of cancer, the method can be for treating diagnosed cancer, preventing recurrence of cancer, or for use in an adjuvant or neoadjuvant setting. In some embodiments, the method comprises providing a T cell engineered to include any CAR polypeptides (and optional antibody reagents or cytokines) described herein or known in the art on the T cell surface; engineering a T cell to include any of the T cell factors described herein (e.g., on the T cell surface); and administering the engineered T cell to the subject. In some embodiments, such methods provide drug-responsive CAR T cells that can, in response to administration of a (e.g., sub-therapeutic) dose of a drug, have their properties (e.g., persistence and/or expansion tendencies, activation state, or phenotypic properties) altered. One aspect of the technology described herein relates to a method of treating cancer, a plasma cell disorder, or an autoimmune disease in a subject in need thereof, the method including: administering the cell of any of the mammalian cells including any of the T cell factors described herein, and any of the CAR polypeptides (and optional antibody reagents or cytokines) described herein or known in the art. In some embodiments of any of aspect, the engineered CAR-T cell is stimulated and/or activated prior to administration to the subject.

In some embodiments, a method of treating cancer, a plasma cell disorder, or an autoimmune disease in a subject in need thereof further comprises administering a drug to the subject, wherein the drug is capable of binding to the T cell factor and inducing degradation of the T cell factor. In some embodiments, the drug binds to a degron domain of the T cell factor, e.g., to a CRBN polypeptide substrate domain. In some embodiments, the drug is a drug described herein, e.g., a thalidomide analog. In some embodiments, the drug is administered in a sub-therapeutic dose. In some embodiments, the drug is administered responsive to the subject experiencing an adverse effect of CAR T therapy (e.g., due to administering the engineered T cell). In some embodiments, the drug is administered responsive to the subject being diagnosed as at risk of experiencing an adverse effect of CAR T therapy (e.g., due to administering the engineered T cell). In some embodiments, the drug is administered as part of a treatment regimen, e.g., a treatment regimen providing for a first period of CAR T therapy (e.g., wherein the CAR T cells are provided expansion and persistence signaling, e.g., the cytokine of a T cell factor) and a second period of CAR T therapy (e.g., wherein the expansion and persistence signaling (e.g., the cytokine of a T cell factor) is not provided).

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior technology or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples, which in no way should be construed as being further limiting.

### EXAMPLES

### Example 1

### Introduction

A general challenge in CAR T cell therapy is inducing CAR T cell activation long enough to effectively treat a cancer in a patient, but also having an ability to decrease activation when the patient show signs of unwanted side effects (e.g. CAR T cell cytotoxicity). One strategy for addressing this problem is developing tools that allow conditional deactivation of CAR T cells in vivo (e.g. in human patients). The disclosure provides examples of T cell factors comprising a degron domain and a cytokine domain. In this Example, an exemplary T cell factor is described that uses a lenalidomide degron tag (a CRBN polypeptide substrate domain) linked to a cytokine domain comprising IL-7 (a T cell activating protein) to conditionally (e.g., drug-inducible) and temporally induce degradation of the T cell factor and its IL-7 (e.g. when a patient show symptoms of cytotoxicity). This exemplary T cell factor is referred to herein as membrane-bound degradable IL-7 (mbdIL7). Without being bound to theory, lenalidomide, at sub-therapeutic concentrations, may binds to the degron domain of mbdIL7, resulting in ubiquitination and subsequent proteasome degradation of mbdIL7. Thus, IL-7 signaling to the CAR T cell can be controlled by introducing mbdIL7 into the CAR T Cells prior to treatment then during treatment administering a sub-therapeutic amount of lenalidomide.

### Results

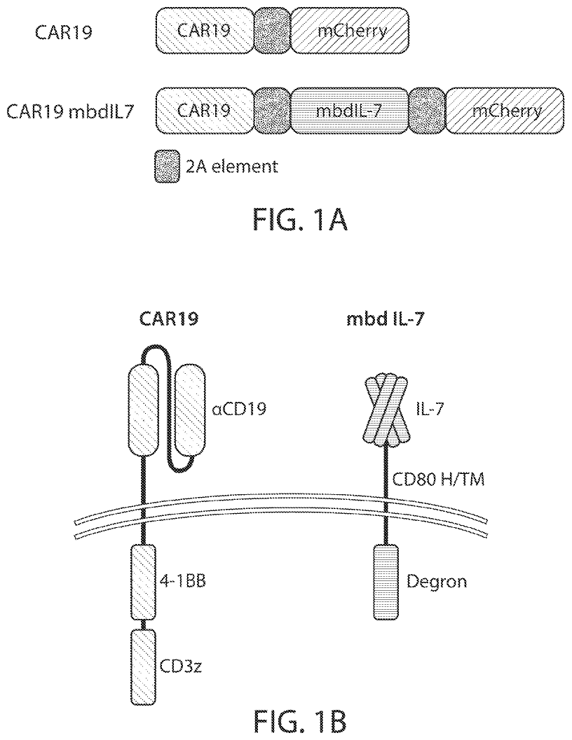

To engineer CAR T cells to deliver a spatially and temporally controllable IL-7 signal, mbdIL7 was designed that could be efficiently degraded with sub-therapeutic doses of FDA-approved lenalidomide. mbdIL7 is encoded by a codon-optimized nucleic acid molecule comprising a sequence encoding human IL-7, a CD80 hinge and transmembrane domain, and a lenalidomide-responsive zinc finger degron domain (FIGS. 1A-1B).

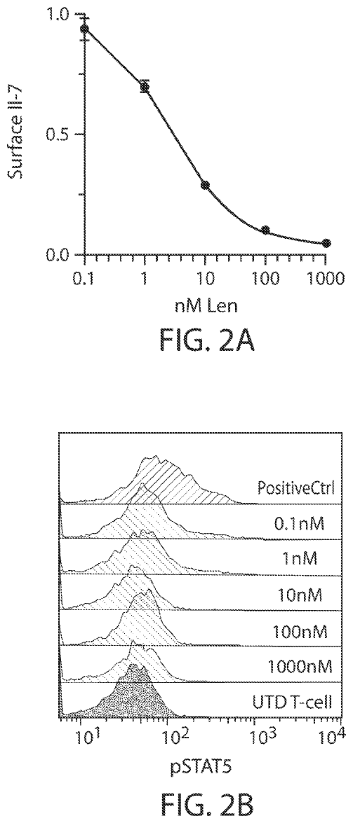

Next it was determined whether expression of the mbdIL7 expression construct could be controlled in vitro by administering increasing amounts of lenalidomide (Len) (FIG. 2A). Results showed that increasing lenalidomide concentration decreases surface expression of mdbIL7 on CD19 CAR-T (CAR19) cells as evidenced by a decrease in surface IL-7 expression and a decrease in pSTAT5 expression. These results indicated that lenalidomide effectively decreases the presence and signaling of mbdIL7 and accordingly is an effective strategy for controlling mdbIL7 expression in a CAR-T cell.

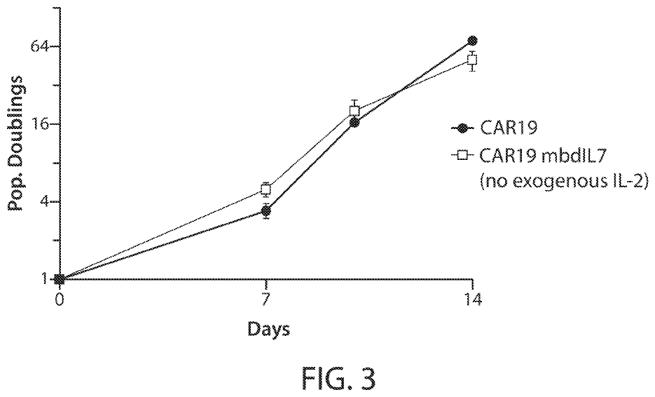

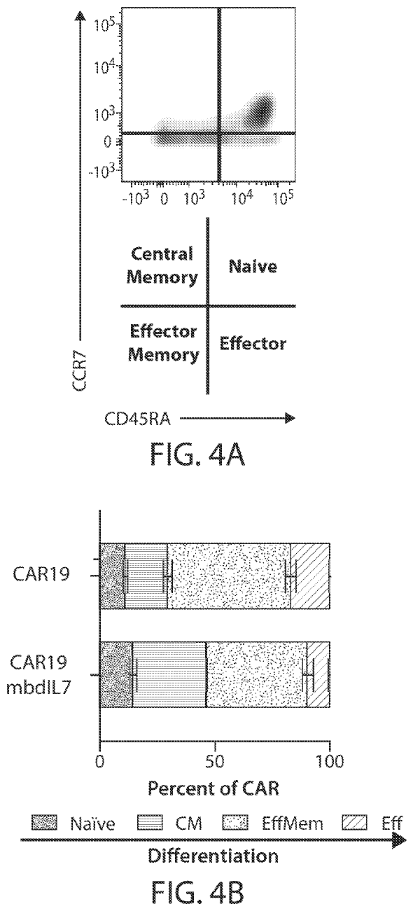

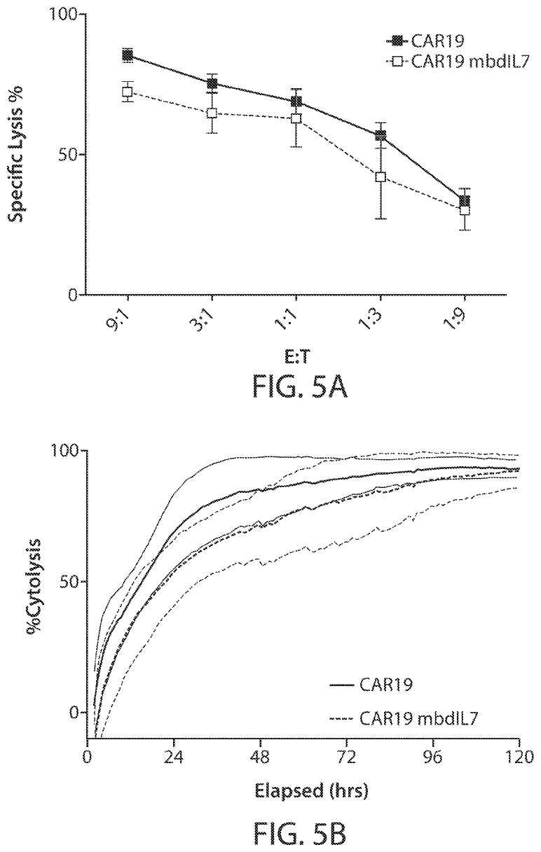

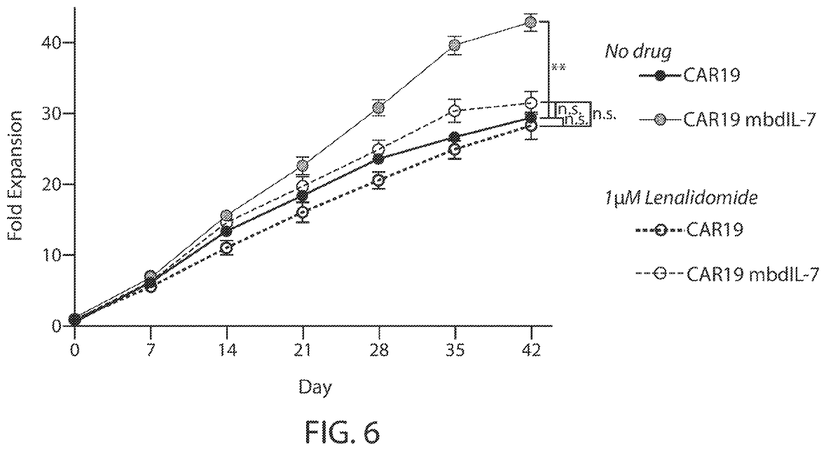

Further studies were performed in vitro to determine how CAR19 T cell properties (e.g. doublings over time, activation, and specific lysis of cancer cells) were affected by mdbIL7. Results showed that CAR19 cell doubling time is not significantly affected by mdbIL7 when CAR19 cells without mbdIL7 are induced with IL-2 (FIG. 3). CAR19 T cell differentiation was determined based on CCR7 and CD45RA expression (FIG. 4A). Results showed that CAR19 mdbIL7 slightly increased the number of central memory cells and decreased the number of effector memory cells and effector cells (FIG. 4B). Results also showed that specific lysis of an exemplary cancer cell line (NALM6 cells) was only slightly decreased by CAR19 cells with mdbIL7 (FIGS. 5A-5B). Results further showed that expansion of CAR19 cells with mdbIL7 was enhanced compared to CAR19 cells without mdbIL7 (FIG. 6) after repeated exposure to cancer cells. Additionally, lenalidomide decreased expansion of CAR19 cells with mdbIL7 to the same level as CAR19 cells without mdbIL7 (FIG. 6C). These results showed that mdbIL7 enhanced long-term proliferation of CAR T cells and that the effects of the exemplary T cell factor can be terminated using lenalidomide.

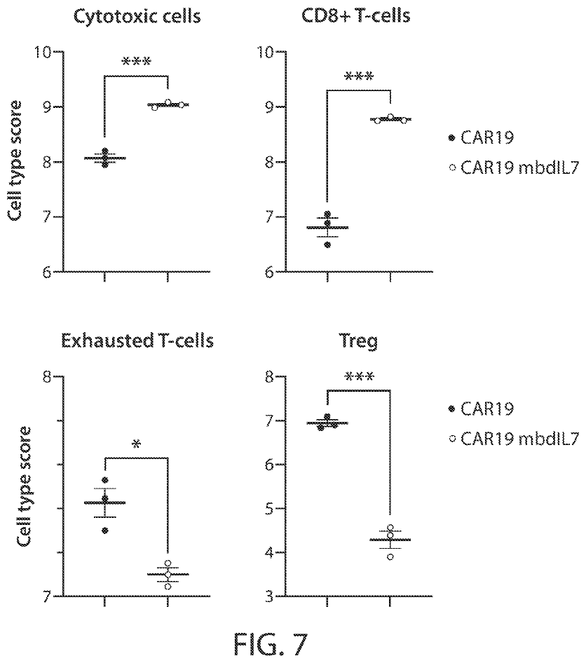

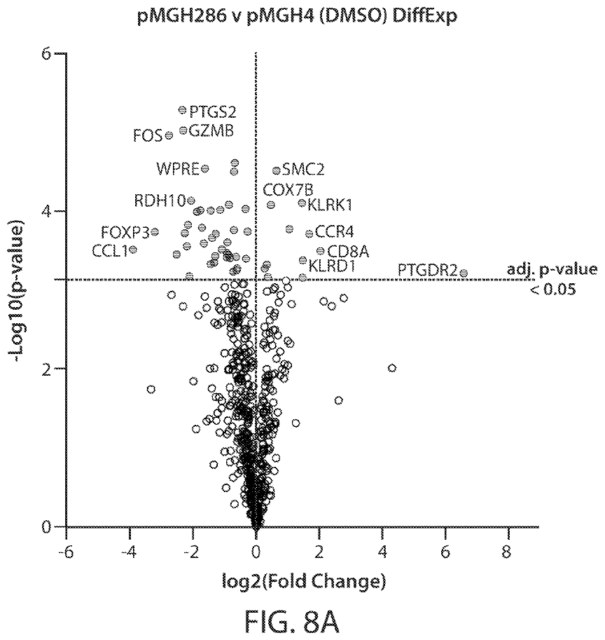

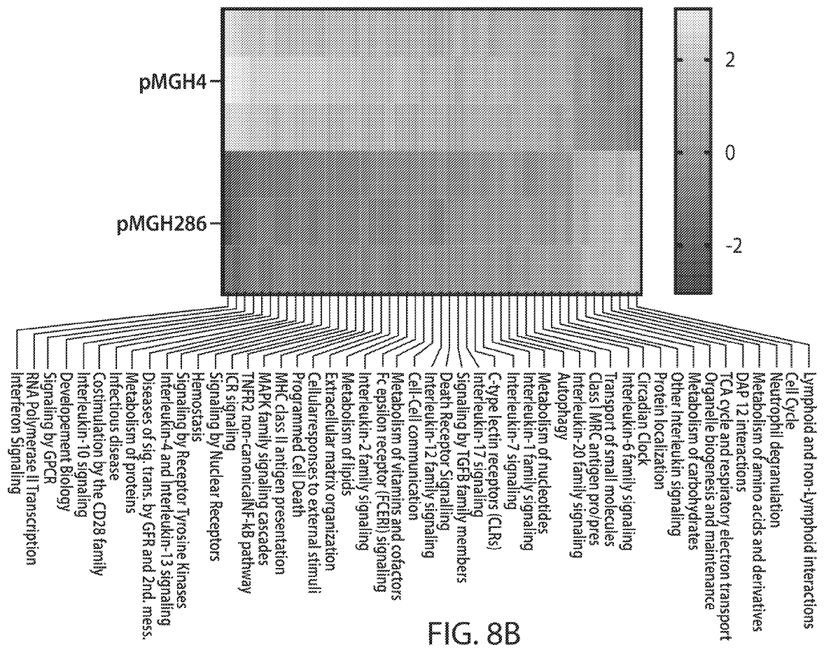

Further, results showed that CAR19 cells with mdbIL7 increase the number of cytotoxic cells and CD8+ T cells and decreased the number of Exhausted T cells, Th1, and Tregs compared to CAR19 cells without mdbTL7 (FIG. 7). Cytotoxicity was based on markers KLRD1, GZMA, NKG7, KLRK1, KLRB1, CTSW, PERF, and GZMB. Exhaustion was based on markers LAG3 and PTGER4. Th1 was based on marker TBX21. Treg was based on marker FOXP3. The results showed that mdbTL7 can alter T cell phenotype, enhancing the effectiveness of CAR T cell therapeutics by increasing the number of cells capable of acting upon a target cell population. Further experiments also detected genes for which expression changed and quantified the change in expression between CAR19 T cells with and without mdbIL7 (FIGS. 8A-8B). Genes that exhibited increased expression included SMC2, COX7B, KLRK1, CCR4, CD8A, KLRD1, and PTGDR2, whereas genes that exhibited decreased expression included PTGS2, GZMB, FOS, WPRE, RDH10, FOXP3, and CCL1.

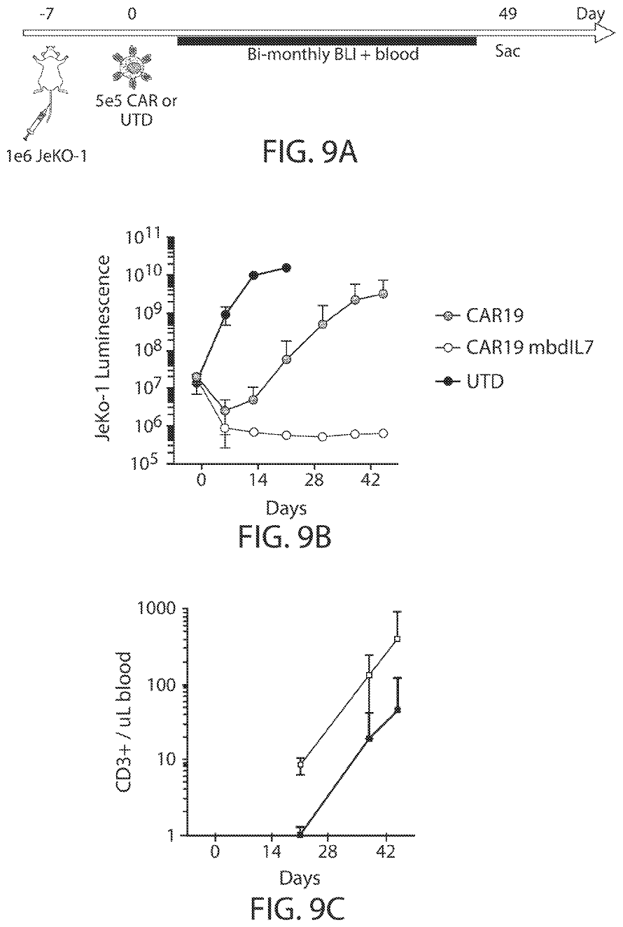

Additional studies were performed to determine the efficacy of CAR19 cells with mdbIL7 in vivo. Mice were injected with JeKO-1 cells (mantle cell lymphoma) and then 7 days later CAR19 cells with or without mdbIL7 were administered (FIG. 9A). Results showed that CAR19 cells with mdbIL7 reduced Jeko-1 cell expression (as measured by luciferase luminescence) by more than 1000-fold by 42 days after treatment (FIG. 9B), whereas the CAR19 cells without mdbTL7 reduced Jeko-1 cell expression for only 7 days before Jeko-1 cell expression increased again. Additionally, there was nearly an order of magnitude more CD3+ cells in the blood of mice treated with CAR19 cells with mdbIL7 compared to mice treated with CAR19 cells without mdbIL7 (FIG. 9C), showing that CAR19 cells with mdbIL7 proliferated and expanded.

### Conclusions

Overall, these results showed that an exemplary T cell factor, membrane-bound degradable IL-7, can be degraded via degron system at sub-therapeutic doses of lenalidomide, mbdIL7 CAR19 have a less differentiated “infusion product”, mbdIL7 CAR19 have superior proliferative capacity upon repeat tumor stimulation (e.g., more cytotoxic, less exhausted, less Tregs), and mbdIL7 CAR19 demonstrate superior in vivo tumor control and CAR-T cell engraftment in xenograft models

### Example 2

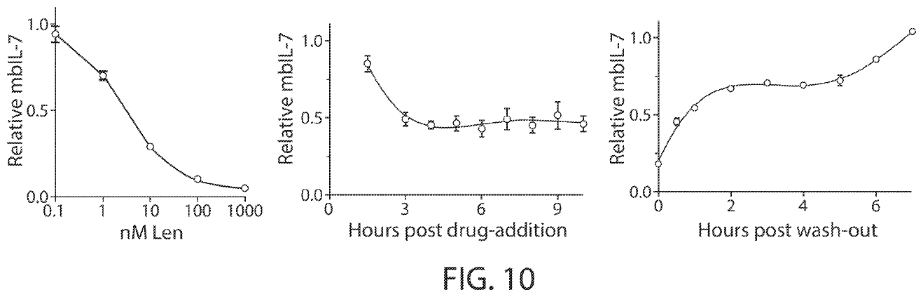

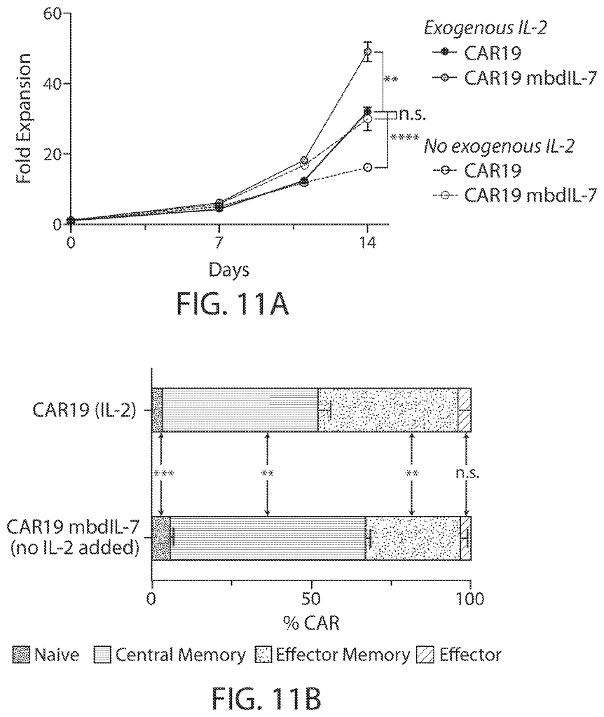

Additional experiments were performed to further determine the characteristics of mbdIL7 CAR19. In vitro studies show that mbl17 is quickly, effectively and reversibly degraded in response to lenalidomide. Specifically, mbdIL7 CAR19 was cultured in increasing concentrations of lenalidomide. Increasing the lenalidomide concentration decreased mbIL17 expression in a dose dependent manner (FIG. 10, left panel). The decrease in mbIL17 concentration took a little bit more than three hours (after administration) to reach a stably decreased level (FIG. 10, middle panel). After lenalidomide was washed out of the cell culture, mdblIL-7 expression reversibly returned to near original expression levels after about 7 hours (FIG. 10, right panel). Overall, these results show that mdbIL17 has dose dependent, time dependent, and reversible effects on mbIL7 expression, which indicates that lenalidomide administration is an effective way to control T cell activation in a reversible manner.

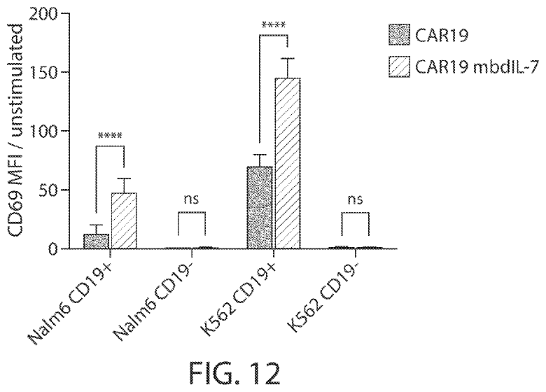

Experiments also showed that mbdIL7 CAR19 T cells do not require exogenous addition of IL-2 for mbdIL7 CAR19 T cell activation and improved phenotype. CAR19 T cells and mbdTL7 CAR19 T cells were cultured with and without exogenous IL-2. Results show that mdbIL7 CAR19 T cells cultured without exogenous IL-2 have significantly more activation that CAR19 T cells cultured without IL2, and about the same level of activation as CAR19 T cells cultured with IL-2. Additionally, CAR19 mdIL7 T cells had more naïve and central memory phenotype T cells that CAR19 T cells with IL-2. Overall, these results indicate that IL-2 cells can be activated without exogenous IL-2, which is advantageous because this suggests that mbdIL17 CAR19 T cells are expected to stay active longer in patients. mbdIL17 CAR19 T cells are expected to stay active longer in patients because they do not require exogenous IL-2 to reach the same level of activation as CAR19 T cells exposed to exogenous IL-2. In agreement with this, FIG. 12 shows that mdbIL7 CAR19 T cells were significantly more active than CAR19 T cells. This was shown for two different types of cancer cells, Nalm6 and K562.

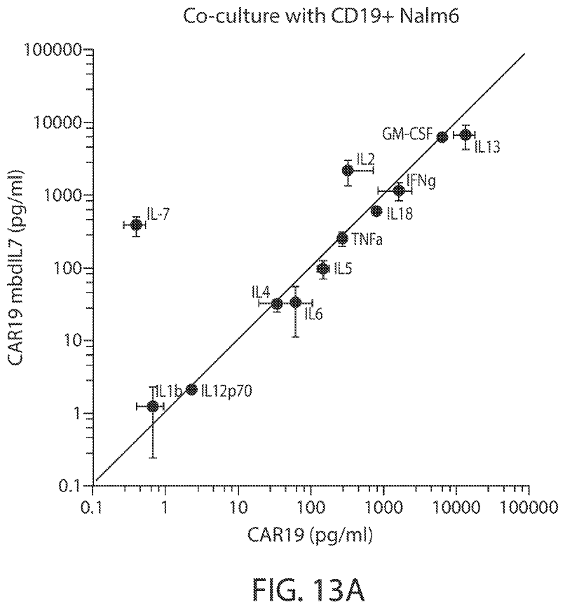

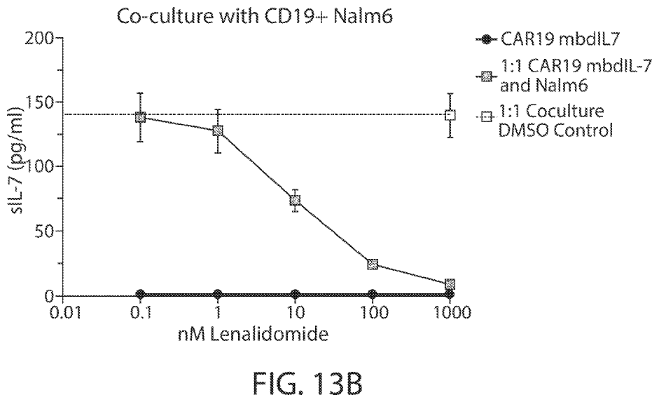

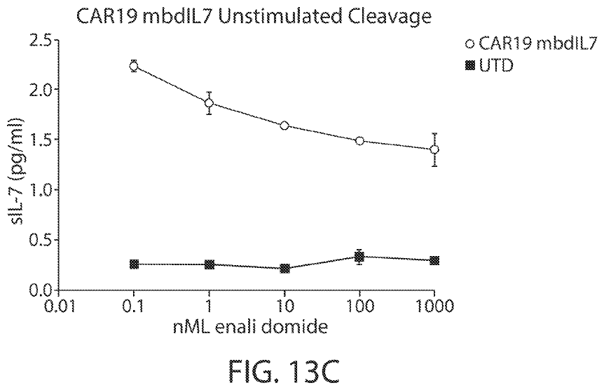

Additional experiments showed that mdbIL7 CAR19 T cells produced more IL7 than CD19 T cells (FIG. 13A). Additionally, cleavage of IL7 was effectively controlled in a dose dependent manner by lenalidomide (FIGS. 13A-13B).

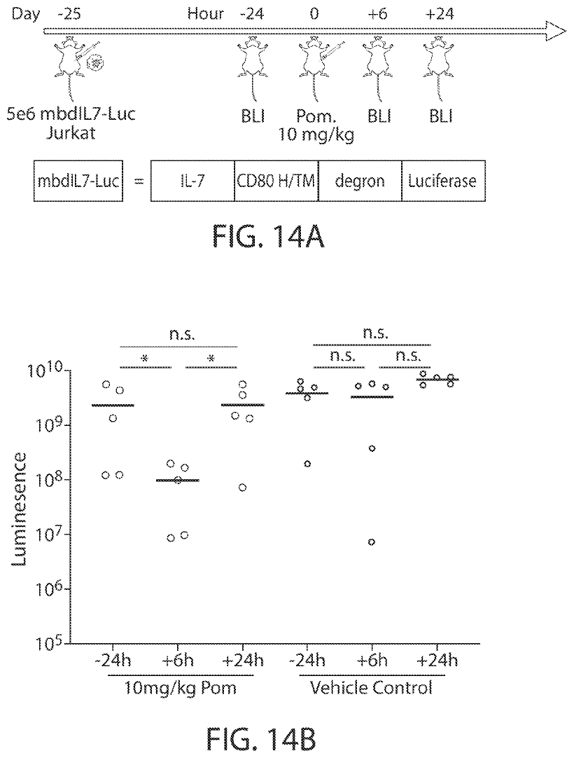

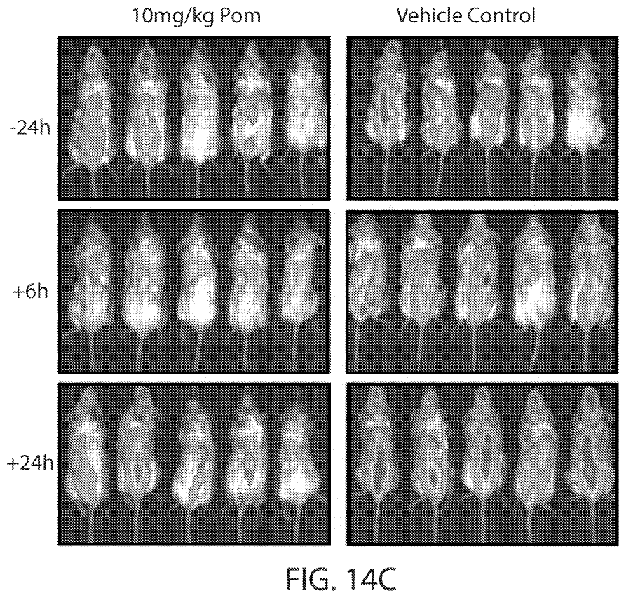

Experiments also showed that pomalidomide (Pom) could reversibly decrease mdbIL7 expression in mice (FIGS. 14A-14C). The mdbIL7 was tagged with luciferase (mdbTL7-Luc), so mdbTL7 expression could be monitored in vivo. Mice were injected with Jurkat cells that were transduced with mdbIL7-Luc. The Jurkat mdbTL7-Luc cells were allowed to engraft for 25 days and then pomalidomide was administered to the mice (FIG. 14A). Results shows that 6 hours after administration of pomalidomide, mdbIL7-luc expression had decreased by more than 10-fold (FIGS. 14B-14C). By 24 hours after pomalidomide administration, mdbIL7-luc expression had returned to levels similar to pre-administration (FIGS. 14B-14C). Overall, these results show that mdbTL7 can be reversibly degraded in vivo, which suggests that mdbIL7 can be used to control CAR T cell activation and proliferation in vivo by administration of thalidomide analogs like pomalidomide and lenalidomide.

1. A drug-responsive T cell factor comprising:
a cytokine domain; and
a degron domain.

2. The T cell factor of claim 1, wherein the cytokine domain comprises a cytokine or portion thereof that promotes proliferation of a T cell.

3. The T cell factor of either of claim 1 or 2, wherein the cytokine domain 10 comprises a cytokine or portion thereof that promotes homeostasis of a T cell.

4. The T cell factor of any one of claims 1-3, wherein the cytokine domain comprises a cytokine or portion thereof that acts as an anti-apoptotic signal for a T cell.

5. The T cell factor of any one of claims 1-4, wherein the cytokine domain comprises a cytokine or portion thereof that promotes homeostatic expansion of a T cell.

6. The T cell factor of any one of claims 1-4, wherein the cytokine domain comprises a cytokine or portion thereof that promotes Th1, Th2, or Th17 differentiation.

7. The T cell factor of any one of claims 1-5, wherein the cytokine domain comprises a cytokine or portion thereof that activates phosphoinositide 3-kinase (PI3K) signaling and/or Janus kinase (JAK)-signal transducer and activator of transcription (STAT) pathway signaling.

8. The T cell factor of any one of claims 1-6, wherein the cytokine domain comprises a cytokine or portion thereof that activates STAT5.

9. The T cell factor of any one of claims 1-7, wherein the cytokine domain comprises an IL-2 family cytokine or a portion thereof.

10. The T cell factor of any one of claims 1-8, wherein the cytokine domain comprises IL-2, IL-4, IL-7, IL-9, IL-15, IL-21, or a portion of any thereof.

11. The T cell factor of any one of claims 1-9, wherein the cytokine domain comprises IL-7 or a portion thereof.

12. The T cell factor of any one of claims 8-10, wherein the cytokine or portion thereof is a human cytokine or a portion of a human cytokine.

13. The T cell factor of any one of claims 1-11, wherein the cytokine domain comprises an amino acid sequence of any one of SEQ ID NOs: 2-5, or a variant thereof.

14. The T cell factor of claim 1, further comprising a transmembrane or hinge domain.

15. The T cell factor of claim 14, wherein the transmembrane or hinge domain is situated between the cytokine domain and the CRBN polypeptide substrate domain.

16. The T cell factor of either one of claims 14-15, wherein the transmembrane or hinge domain comprises a CD80 transmembrane/hinge domain.

17. The T cell factor of claim 16, wherein the transmembrane or hinge domain comprises an amino acid sequence of any one of SEQ ID NOs: 1 or 47-48, or a variant thereof.

18. The T cell factor of any one of claims 1-17, wherein the degron domain comprises a cereblon (CRBN) polypeptide substrate domain capable of binding CRBN in response to the drug.

19. The T cell factor of claim 18, wherein the CRBN polypeptide substrate domain binding to CRBN promotes degradation of the T cell factor.

20. The T cell factor of claim 19, wherein degradation is mediated by the ubiquitin-pathway.

21. The T cell factor of any one of claims 1-17, further comprising the drug.

22. The T cell factor of any one of claims 1-17, wherein the drug is a small molecule drug.

23. The T cell factor of any one of claims 1-22, wherein the drug is an FDA-approved drug.

24. The T cell factor of any one of claims 1-23, wherein the drug can be administered to a human subject in a clinical setting.

25. The T cell factor of any one of claims 1-24, wherein the drug is a thalidomide analog.

26. The T cell factor of any one of claims 1-25, wherein the drug is an immunomodulatory imide drug (IMiD).

27. The T cell factor of any one of claims 1-26, wherein the drug is selected from the group consisting of thalidomide, lenalidomide and pomalidomide.

28. The T cell factor of any one of claims 1-18, wherein the CRBN polypeptide substrate domain is selected from the group consisting of IKZF1, IKZF3, CK1α, ZFP91, GSPT1, MEIS2, GSS, E4F1, ZN276, ZN517, ZN582, ZN653, ZN654, ZN692, ZN787, ZN827, or a fragment thereof that is capable of drug-inducible binding a CRBN polypeptide.

29. The T cell factor of claim 28, wherein the CRBN polypeptide domain comprises IKZF3 or a fragment thereof that is capable of drug-inducible binding a CRBN polypeptide.

30. The T cell factor of claim 29, wherein the CRBN polypeptide domain comprises amino acids 130-145, amino acids 169-189, or amino acids 130-189 of IKZF3 (SEQ ID NO: 21).

31. The T cell factor of any one of claims 1-27, wherein the CRBN polypeptide substrate domain comprises a hybrid fusion polypeptide comprised of ten or more residues of a non-IKZF3 C2H2 zinc finger degron sequence flanked by an N-terminal IKZF3 degron sequence and a C-terminal IKZF3 degron sequence.

32. The T cell factor of claim 31, wherein the N-terminal IKZF3 degron sequence comprises or is amino acids 130-145 (SEQ ID NO: 23) of IKZF3 and/or wherein the C-terminal IKZF3 degron sequence comprises or is amino acids 169-189 (SEQ ID NO: 25) of IKZF3.

33. The T cell factor of either one of claim 31 or 32, wherein the non-IKZF3 C2H2 zinc finger degron sequence is a ZFP91 sequence.

34. The T cell factor of any one of claims 1-33, wherein the CRBN polypeptide 15 substrate domain comprises the amino acid sequence of SEQ ID NO: 22.

35. The T cell factor of any one of claims 1-33, wherein the CRBN polypeptide substrate domain comprises the amino acid sequence of SEQ ID NO: 27.

36. The T cell factor of any one of claims 1-35, comprising an amino acid sequence of SEQ ID NO: 29 or a variant thereof.

37. A nucleic acid molecule comprising a sequence encoding the T cell factor of any one of claims 1-36.

38. The nucleic acid molecule of claim 37, comprising a first polynucleotide encoding the T cell factor and a second polynucleotide encoding a chimeric antigen receptor (CAR).

39. The nucleic acid molecule of claim 38, wherein the CAR comprises an extracellular antigen-binding domain, a transmembrane domain (TMD), a co-stimulatory domain, and a signaling domain.

40. The nucleic acid molecule of any one of claims 37-39, wherein the nucleic acid molecule is a vector.

41. The nucleic acid molecule of claim 40, wherein the vector is a plasmid or a viral vector.

42. The nucleic acid molecule of claim 41, wherein the viral vector is a lentiviral or adeno-associated viral vector.

43. A mammalian cell comprising the T cell factor of any one of claims 1-36 or the nucleic acid molecule of any one of claims 37-42.

44. The mammalian cell of claim 43, wherein the mammalian cell overexpresses a CRBN polypeptide.

45. The mammalian cell of claim 44, wherein the overexpressed CRBN polypeptide is targeted to the plasma membrane with a targeting sequence derived from LAT, PAG, LCK, FYN, LAX, CD2, CD3, CD4, CDS, CD7, CD8a, PDl, SRC, or LYN.

46. The mammalian cell of either one of claim 44 or 45, wherein the local concentration of the ubiquitin ligase CRL4^{CRBN} is increased at the plasma membrane, as compared to an appropriate control.

47. The mammalian cell of any one of claims 43-46, wherein the mammalian cell is a T cell.

48. The mammalian cell of any one of claims 43-46, wherein the cell is selected from the group consisting of a B cell, plasma cell, NK cell, NKT cell, innate lymphoid cell, macrophage, dendritic cell, monocyte, neutrophil, basophil, eosinophil, mast cell, hematopoietic progenitor cell, hematopoietic stem cell, other adult stem cell such as neural, cornea, muscle, skin, small intestine, colon, bone, mesenchyme, embryonic stem cell and an induced pluripotent stem cell.

49. The mammalian cell of claim 47, wherein the cell is a chimeric antigen receptor (CAR) T cell.

50. The mammalian cell of claim 49, wherein the cell comprises a CAR comprising: an extracellular antigen-binding domain, a transmembrane domain (TMD), a co-stimulatory domain, and a signaling domain.

51. The mammalian cell of claim 50, wherein the cell comprises a first polynucleotide encoding the T cell factor and a second polynucleotide encoding the CAR.

52. The mammalian cell of claim 51, wherein the first polynucleotide and second polynucleotide are on the same nucleic acid molecule.

53. The mammalian cell of claim 51, wherein the first polynucleotide and second polynucleotide are on different nucleic acid molecules.

54. The mammalian cell of claim 52 or nucleic acid of any one of claims 38-42, wherein the CAR and the T cell factor are produced in the form of a single polypeptide, which is cleaved to generate separate CAR and therapeutic agent molecules.

55. The mammalian cell or nucleic acid of claim 54, wherein the single polypeptide comprises a cleavable moiety between the CAR and the T cell factor.

56. The mammalian cell or nucleic acid of claim 55, wherein the cleavable moiety comprises: a 2A peptide, a 2A ribosomal skip sequence, or an IRES.

57. The mammalian cell or nucleic acid of claim 56, wherein the 2A peptide comprises P2A or T2A.

58. The mammalian cell of any one of claims 50-57 or nucleic acid of any one of claims 38-42, wherein the CAR and the T cell factor are each constitutively expressed.

59. The mammalian cell of any one of claims 50-58 or nucleic acid of any one of claims 38-42, wherein expression of the CAR and the T cell factor is driven by an elongation factor-1 alpha (EF1α) promoter.

60. The mammalian cell of any one of claims 43-53 or nucleic acid of any one of claims 38-42, wherein the T cell factor is expressed under the control of an inducible promoter, which is optionally inducible by T cell receptor or CAR signaling.

61. The mammalian cell or nucleic acid of claim 60, wherein the inducible promoter comprises the NFAT promoter.

62. The mammalian cell of any one of claims 50-61 or nucleic acid of any one of claims 38-42, wherein the CAR is expressed under the control of a constitutive promoter and the T cell factor is expressed under the control of an inducible promoter, which is optionally inducible by T cell receptor or CAR signaling.

63. The mammalian cell of any one of claims 50-62 or nucleic acid of any one of claims 39-42, wherein the extracellular antigen-binding domain of the CAR comprises an antibody, a single chain antibody, a single domain antibody, or a ligand.

64. The mammalian cell of any one of claims 50-63 or nucleic acid of any one of claims 39-42, wherein the extracellular antigen-binding domain binds to a tumor-associated antigen.

65. The mammalian cell or nucleic acid of claim 64, wherein the tumor-associated antigen is selected from the group consisting of EGFR, EGFRvIII, CD19, CD37, BCMA, CEA, immature laminin receptor, TAG-72, HPV E6 and E7, BING-4, calcium-activated chloride channel 2, cyclin B1, 9D7, Ep-CAM, EphA3, her2/neu, telomerase, mesothelin, SAP-1, Survivin, BAGE family, CAGE family, GAGE family, MAGE family, SAGE family, XAGE family, NY-ESO-1/LAGE-1, PRAME, SSX-2, Melan-A/MART-1, gp100/pmel17, tyrosinase, TRP-1/-2, MC1R, BRCA1/2, CDK4, MART-2, p53, Ras, MUC1, TGF-βRII, IL-15, IL13Ra2, or CSF1R.

66. The mammalian cell of any one of claims 43-65 or nucleic acid of any one of claims 39-42, wherein the transmembrane domain of the CAR comprises a CD8 hinge/transmembrane domain, which optionally comprises the sequence of SEQ ID NO: 47, or a variant thereof.

67. The mammalian cell of any one of claims 43-66 or nucleic acid of any one of claims 39-42, wherein the signaling domain comprises a CD3ζ domain, a CD3 gamma domain, a CD3 delta domain, a CD3 epsilon domain, a FcR gamma domain, a FcR beta domain, a CDS domain, a CD79a domain, a CD79b domain, a CD66d domain, a CD4 domain, a CD8 domain, a Dap10 domain, and a Dap-12 domain, wherein the signaling domain optionally comprises the sequence of SEQ ID NO: 50, or a variant thereof.

68. The mammalian cell of any one of claims 43-67 or nucleic acid of any one of claims 39-42, wherein the co-stimulatory domain comprises a CD28 co-stimulatory domain or a 4-1BB co-stimulatory domain, wherein the co-stimulatory domain optionally comprises the sequence of SEQ ID NO: 49 or a variant thereof.

69. A pharmaceutical composition comprising the mammalian cell of any one of claims 43-68 or nucleic acid of any one of claims 39-42.

70. A method for treating a subject with a chimeric antigen receptor (CAR) cellular therapy, the method comprising administering to the subject a drug-responsive CAR T cell, wherein the CAR T cell is a CAR T cell of claim 49.

71. The method of claim 70, further comprising administering the drug.

72. The method of either one of claim 70 or 71, further comprising identifying a CAR cellular therapy side effect in the subject.

73. The method of claim 72, further comprising administering the drug after the CAR cellular therapy side effect is identified in the subject.

74. The method of any one of claims 70-73, wherein the drug is a small molecule drug.

75. The method of any one of claims 70-74, wherein the drug is an FDA-approved drug.

76. The method of any one of claims 70-75, wherein the drug can be administered to a human subject in a clinical setting.

77. The method of any one of claims 72-76, wherein the drug is a thalidomide analog.

78. The method of any one of claims 72-77, wherein the drug is an immunomodulatory imide drug (IMiD).

79. The method of any one of claims 72-78, wherein the drug is selected from the group consisting of thalidomide, lenalidomide and pomalidomide.

80. The method of any one of claims 72-79, wherein the subject has or is at risk of developing cancer.

81. The method of claim 80, wherein the cancer is glioblastoma, glioma, leukemia, lymphoma, multiple myeloma, or a solid tumor.

82. The method of claim 81, wherein the leukemia is acute myeloid leukemia (AML), chronic myeloid leukemia (CML), acute lymphocytic leukemia (ALL), or chronic lymphocytic leukemia (CLL);
wherein the lymphoma is diffuse large B-cell lymphoma (DLBCL), follicular lymphoma, chronic lymphocytic leukemia (CLL), small lymphocytic lymphoma (SLL), mantle cell lymphoma (MCL), marginal zone lymphomas, Burkitt lymphoma, hairy cell leukemia (HCL), and T cell lymphoma (e.g., peripheral T cell lymphoma (PTCL), including cutaneous T cell lymphoma (CTCL) and anaplastic large cell lymphoma (ALCL)); or
wherein the solid tumor is adrenocortical tumor, alveolar soft part sarcoma, carcinoma, chondrosarcoma, colorectal carcinoma, desmoid tumors, desmoplastic small round cell tumor, endocrine tumors, endodermal sinus tumor, epithelioid hemangioendothelioma, Ewing sarcoma, germ cell tumors (solid tumor), giant cell tumor of bone and soft tissue, hepatoblastoma, hepatocellular carcinoma, melanoma, nephroma, neuroblastoma, non-rhabdomyosarcoma soft tissue sarcoma (NRSTS), osteosarcoma, paraspinal sarcoma, renal cell carcinoma, retinoblastoma, rhabdomyosarcoma, synovial sarcoma, or Wilms tumor.

83. A method of producing a drug-responsive chimeric antigen receptor (CAR) T cell, comprising:
contacting a CAR T cell with a nucleic acid molecule of any one of claims 37-42, thereby producing a drug-responsive CAR T cell.

84. The method of claim 83, wherein contacting comprises transfection, transduction, and/or electroporation of the nucleic acid molecule.

85. The method of either one of claim 83 or 84, wherein the nucleic acid molecule is integrated into the genome of the CAR T cell.

86. The method of any one of claims 83-85, wherein the T cell factor encoded by the nucleic acid molecule is expressed in the drug-responsive CAR T cell.

87. The method of any one of claims 83-86, further comprising providing the CAR T cell.

88. The method of claim 87, wherein providing the CAR T cell comprises contacting a T cell with a nucleic acid molecule encoding the CAR.

89. The method of claim 88, wherein the nucleic acid molecule encoding the CAR is a vector.

90. The method of claim 89, wherein the vector is a plasmid or a viral vector.

91. The method of claim 90, wherein the viral vector is a lentiviral or adeno-associated viral vector.

92. The method of any one of claims 88-91, wherein the CAR is encoded upon the same nucleic acid molecule that encodes the T cell factor.

93. The method of any one of claims 88-91, wherein the nucleic acid molecule encoding the CAR and the nucleic acid molecule encoding the T cell factor are different nucleic acid molecules.

94. The method of any one of claims 83-93, further comprising selecting for a drug-responsive CAR T cell.

95. The method of claim 94, wherein selecting comprises using cell sorting, e.g., Fluorescence Activated Cell Sorting (FACS).

96. The method of either one of claim 94 or 95, wherein selecting selects for cells comprising a nucleic acid molecule encoding the T cell factor.

97. The method of any one of claims 94-96, wherein selecting selects for cells comprising a nucleic acid molecule encoding the CAR.

98. The method of any one of claims 83-97, further comprising contacting a T cell with a nucleic acid molecule comprising a sequence encoding a CRBN polypeptide.

99. The method of claim 98, wherein the sequence encoding a CRBN polypeptide is operably linked to a promoter configured to overexpress the CRBN polypeptide in the T cell.

100. The method of either one of claim 98 or 99, wherein the CRBN polypeptide encoded by the nucleic acid molecule is targeted to the plasma membrane with a targeting sequence derived from LAT, PAG, LCK, FYN, LAX, CD2, CD3, CD4, CDS, CD7, CD8a, PDl, SRC, or LYN.

101. The method of any one of claims 98-100, wherein the local concentration of the ubiquitin ligase CRL4^{CRBN} is increased at the plasma membrane, as compared to an appropriate control.

102. The method of any one of claims 98-101, wherein the nucleic acid molecule encoding the CRBN polypeptide is a vector.

103. The method of claim 102, wherein the vector is a plasmid or a viral vector.

104. The method of claim 103, wherein the viral vector is a lentiviral or adeno-associated viral vector.

105. The method of any one of claims 98-104, wherein the CRBN polypeptide is encoded upon the same nucleic acid molecule that encodes the T cell factor.

106. The method of any one of claims 98-104, wherein the CRBN polypeptide is encoded upon the same nucleic acid molecule that encodes the CAR.

107. The method of any one of claims 98-106, wherein the CRBN polypeptide is encoded upon the same nucleic acid molecule that encodes the T cell factor and the CAR.

108. The method of any one of claims 98-104, wherein the nucleic acid molecule encoding the CRBN polypeptide, the nucleic acid encoding the CAR, and the nucleic acid molecule encoding the T cell factor are different nucleic acid molecules.

109. The method of any one of claims 94-108, selecting selects for cells comprising a nucleic acid molecule encoding the CRBN polypeptide.


SEQUENCE LISTING (57 sequences)

SEQ ID NO: 1 - protein, 58 aa, synthetic construct
WNTTKQEHFPDNLLPSWAITLISVNGIFVICCLTYCFAPRCRERRRNERLRRESVRPV

SEQ ID NO: 2 - protein, 177 aa, synthetic construct
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 3 - protein, 152 aa, synthetic construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 4 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 5 - protein, 562 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 6 - protein, 519 aa, synthetic construct
MDADEGQDMSQVSGKESPPVSDTPDEGDEPMPIPEDLSTTSGGQQSSKSDRVVASNVKVETQSDEENGRACEMNGEECAEDLRMLDASGEKMNGSHRDQGSSALSGVGGIRLPNGKLKCDICGIICIGPNVLMVHKRSHTGERPFQCNQCGASFTQKGNLLRHIKLHSGEKPFKCHLCNYACRRRDALTGHLRTHSVGKPHKCGYCGRSYKQRSSLEEHKERCHNYLESMGLPGTLYPVIKEETNHSEMAEDLCKIGSERSLVLDRLASNVAKRKSSMPQKFLGDKGLSDTPYDSSASYEKENEMMKSHVMDQAINNAINYLGAESLRPLVQTPPGGSEVVPVISPMYQLHKPLAEGTPRSNHSAQDSAVENLLLLSKAKLVPSEREASPSNSCQDSTDTESNNEEQRSGLIYLTNHIAPHARNGLSLKEEHRAYDLLRAASENSQDALRVVSTSGEQMKVYKCEHCRVLFLDHVMYTIHMGCHGFRDPFECNMCGYHSQDRYEFSSHITRGEHRFHMS

SEQ ID NO: 7 - protein, 337 aa, synthetic construct
MASSSGSKAEFIVGGKYKLVRKIGSGSFGDIYLAINITNGEEVAVKLESQKARHPQLLYESKLYKILQGGVGIPHIRWYGQEKDYNVLVMDLLGPSLEDLFNFCSRRFTMKTVLMLADQMISRIEYVHTKNFIHRDIKPDNFLMGIGRHCNKLFLIDFGLAKKYRDNRTRQHIPYREDKNLTGTARYASINAHLGIEQSRRDDMESLGYVLMYFNRTSLPWQGLKAATKKQKYEKISEKKMSTPVEVLCKGFPAEFAMYLNYCRGLRFEEAPDYMYLRQLFRILFRTLNHQYDYTFDWTMLKQKAAQQAASSSGQGQQAQTPTGKQTDKTKSNMKGF

SEQ ID NO: 8 - protein, 567 aa, synthetic construct
MPGETEEPRPPEQQDQEGGEAAKAAPEEPQQRPPEAVAAAPAGTTSSRVLRGGRDRGRAAAAAAAAAVSRRRKAEYPRRRRSSPSARPPDVPGQQPQAAKSPSPVQGKKSPRLLCIEKVTTDKDPKEEKEEEDDSALPQEVSIAASRPSRGWRSSRTSVSRHRDTENTRSSRSKTGSLQLICKSEPNTDQLDYDVGEEHQSPGGISSEEEEEEEEEMLISEEEIPFKDDPRDETYKPHLERETPKPRRKSGKVKEEKEKKEIKVEVEVEVKEEENEIREDEEPPRKRGRRRJDDKSPRLPKRRKKPPIQYVRCEMEGCGTVLAHPRYLQHHIKYQHLLKKKYVCPHPSCGRLFRLQKQLLRHAKHHTDQRDYICEYCARAFKSSHNLAVHRMIHTGEKPLQCEICGFTCRQKASLNWHMKKHDADSFYQFSCCGKKFEKKDSVVAHKAKSHPEVLIAEALAANAGALITSTDILGTPESLTQPSDGQGLPLLPEPLGNSTSGECLLLEAEGMSKSYCSGTERVSLMADGKIFVGSGSSGGTEGLVMNSDILGATTEVLIEDSDSAGP

SEQ ID NO: 9 - protein, 633 aa, synthetic construct
MDPGSGGGGGGGGSSSGSSSSDSAPDCWDQADMEAPGPGPCGGGGSLAAAAEAQRENLSAAFSRQLNVNAKPFVPNVHAAEFVPSFLRCPAAPPPPAGGAANNHGAGSGAGGRAAPVESSQEEQSLCEGSNSAVSMELSEPIENGETEMSPEESWEHKEEISEAEPGGGSLGDGRPPEESAFIEMMEEEEEIPKPKSVVAPPGAPKKEHVNVVFIGHVDAGKSTIGGQFYLTGMVDKRTLEKYEREAKEKNRETWYLSWALDTNQEERDKGKTVEVGRAYFETEKKHFTILDAPGHKSFVPNMIGGASQADLAVLVISARKGEFETGFEKGGQTREHAMLAKTAGVKHLIVLINKMDDPTVNWSNERYEECKEKLVPFLKKVGFNPKKDIHFMPCSGLTGANLKEQSDFCPWYIGLPFIPYLDNLPNFNRSVDGPIRLPIVDKYKDMGTVVLGKLESGSICKGQQLVMMPNKHNVEVLGILSDDVETDTVAPGENLKIRLKGIEEEEILPGFILCDPNNLCHSGRTFDAQIVIIEHKSIICPGYNAVLHIHTCIEEVEITALICLVDKKSGEKSKTRPRFVKQDQVCIARLRTAGTICLETFKDFPQMGRFTLRDEGKTIAIGKVLKLVPEKD

SEQ ID NO: 10 - protein, 394 aa, synthetic construct
MAQRYDELPHYGGMDGVGVPASMYGDPHAPRPIPPVHHLNHGPPLHATQHYGAHAPHPNVMPASMGSAVDALKRDKDAIYGHPLFPLLALVFEKCELATCTPREPGVAGGDVCSSDSFEDIAVFAKQVRAEKPLFSSPELDMMIQAIQVLRFHLLELEKVHELCDFCHRYISCLKGKMPIDLVIDERDGSSKSDHEELSGSSTNLADHNPSSWRDHDDATSTHSAGTPGPSSGGHASQSGDNSSEQGDGLDNSVASPGTGDDDDPDKDKKRQKKRGIFPKVATNFRAWLFQHLTHPYPSEEQKKQLAQDTGLTILQVNNWFINARRRIVQPMIDQSRAGFLLDPSVSQGAAYSPEGQPMGSFVLDGQQHMGIRPAGPMSGMGMNMGMDGQWHYM

SEQ ID NO: 11 - protein, 473 aa, synthetic construct
MATNWGSLLQDKQQLEELARQAVDRALAEGVLLRTSQEPTSSEWSYAPFTLFPSLVPSALLEQAYAVQMDFNLLVDAVSQNAAFLEQTLSSTIKQDDFTARLFDIHKQVLKEGIAQTVFLGLNRSDYMFQRSADGSPALKQIEINTISASFGGLASRTPAVHRHVLSVLSKTKEAGKILSNNPSKGLALGIAKAWELYGSPNALVLLIAQEKERNIFDQRAIENELLARNIHVIRRTFEDISEKGSLDQDRRLFVDGQEIAVVYFRDGYMPRQYSLQNWEARLLLERSHAAKCPDIATQLAGTKKVQQELSRPGMLEMLLPGQPEAVARLRATFAGLYSLDVGEEGDQAIAEALAAPSRFVLKPQREGGGNNLYGEEMVQALKQLKDSEERASYILMEKIEPEPFENCLLRPGSPARVVQCISELGIFGVYVRQEKTLVMNKHVGHLLRTKAIEHADGGVAAGVAVLDNPYPV

SEQ ID NO: 12 - protein, 23 aa, synthetic construct
HECKLCGASFRTKGSLIRHHRRH

SEQ ID NO: 13 - protein, 23 aa, synthetic construct
LQCEVCGFQCRQRASLKYHMTKH

SEQ ID NO: 14 - protein, 23 aa, synthetic construct
YRCRACGRACSRLSTLIQHQKVH

SEQ ID NO: 15 - protein, 23 aa, synthetic construct
YQCKVCGRAFKRVSHLTVHYRIH

SEQ ID NO: 16 - protein, 23 aa, synthetic construct
LQCEICGYQCRQRASLNWHMKKH

SEQ ID NO: 17 - protein, 23 aa, synthetic construct
FACVICGRKFRNRGLMQKHLKNH

SEQ ID NO: 18 - protein, 23 aa, synthetic construct
LQCEICGFTCRQKASLNWHQRKH

SEQ ID NO: 19 - protein, 23 aa, synthetic construct
FVCPRCGRGFSQPKSLARHLRLH

SEQ ID NO: 20 - protein, 23 aa, synthetic construct
FQCPICGLVIKRKSYWKRHMVIH

SEQ ID NO: 21 - protein, 509 aa, synthetic construct
MEDIQTNAELKSTQEQSVPAESAAVLNDYSLTKSHEMENVDSGEGPANEDEDIGDDSMKVKDEYSERDENVLKSEPMGNAEEPEIPYSYSREYNEYENIKLERHVVSFDSSRPTSGKMNCDVCGLSCISFNVLMVHKRSHTGERPFQCNQCGASFTQKGNLLRHIKLHTGEKPFKCHLCNYACQRRDALTGHLRTHSVEKPYKCEFCGRSYKQRSSLEEHKERCRTFLQSTDPGDTASAEARHIKAEMGSERALVLDRLASNVAKRKSSMPQKFIGEKRHCFDVNYNSSYMYEKESELIQTRMMDQAINNAISYLGAEALRPLVQTPPAPTSEMVPVISSMYPIALTRAEMSNGAPQELEKKSIHLPEKSVPSERGLSPNNSGHDSTDTDSNHEERQNHIYQQNHMVLSRARNGMPLLKEVPRSYELLKPPPICPRDSVKVINKEGEVMDVYRCDHCRVLFLDYVMFTIHMGCHGFRDPFECNMCGYRSHDRYEFSSHIARGEHRALLK

SEQ ID NO: 22 - protein, 60 aa, synthetic construct
FNVLMVHKRSHTGERPLQCEICGFTCRQKGNLLRHIKLHTGEKPFKCHLCNYACQRRDAL

SEQ ID NO: 23 - protein, 16 aa, synthetic construct
FNVLMVHKRSHTGERP

SEQ ID NO: 24 - protein, 16 aa, synthetic construct
FNVLMVHRRSHTGERP

SEQ ID NO: 25 - protein, 21 aa, synthetic construct
TGEKPFKCHLCNYACQRRDAL

SEQ ID NO: 26 - protein, 21 aa, synthetic construct
TGERPFRCHLCNYACQRRDAL

SEQ ID NO: 27 - protein, 60 aa, synthetic construct
FNVLMVHKRSHTGERPFQCNQCGASFTQKGNLLRHIKLHTGEKPFKCHLCNYACQRRDAL

SEQ ID NO: 28 - protein, 70 aa, synthetic construct
GSGSGSGSGGFNVLMVHKRSHTGERPLQCEICGFTCRQKGNLLRHIKLHTGEKPFKCHLCNYACQRRDAL

SEQ ID NO: 29 - protein, 312 aa, synthetic construct
MLLLVTSLLLCELPHPAFLLIPEQKLISEEDLDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHWNTTKQEHFPDNLLPSWAITLISVNGIFVICCLTYCFAPRCRERRRNERLRRESVRPVGSGSGSGSGGFNVLMVHKRSHTGERPLQCEICGFTCRQKGNLLRHIKLHTGEKPFKCHLCNYACQRRDAL

SEQ ID NO: 30 - protein, 22 aa, synthetic construct
MLLLVTSLLLCELPHPAFLLIP

SEQ ID NO: 31 - protein, 10 aa, synthetic construct
EQKLISEEDL

SEQ ID NO: 32 - protein, 825 aa, synthetic construct
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMLLLVTSLLLCELPHPAFLLIPEQKLISEEDLDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHWNTTKQEHFPDNLLPSWAITLISVNGIFVICCLTYCFAPRCRERRRNERLRRESVRPVGSGSGSGSGGFNVLMVHKRSHTGERPLQCEICGFTCRQKGNLLRHIKLHTGEKPFKCHLCNYACQRRDAL

SEQ ID NO: 33 - protein, 825 aa, synthetic construct
MLLLVTSLLLCELPHPAFLLIPEQKLISEEDLDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHWNTTKQEHFPDNLLPSWAITLISVNGIFVICCLTYCFAPRCRERRRNERLRRESVRPVGSGSGSGSGGFNVLMVHKRSHTGERPLQCEICGFTCRQKGNLLRHIKLHTGEKPFKCHLCNYACQRRDALGSGATNFSLLKQAGDVEENPGPMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 1085 aa, synthetic construct
MLLLVTSLLLCELPHPAFLLIPEQKLISEEDLDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHWNTTKQEHFPDNLLPSWAITLISVNGIFVICCLTYCFAPRCRERRRNERLRRESVRPVGSGSGSGSGGFNVLMVHKRSHTGERPLQCEICGFTCRQKGNLLRHIKLHTGEKPFKCHLCNYACQRRDALGSGATNFSLLKQAGDVEENPGPMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGGGGEGRGSLLTCGDVEENPGPRMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 35 - protein, 1085 aa, synthetic construct
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMLLLVTSLLLCELPHPAFLLIPEQKLISEEDLDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHWNTTKQEHFPDNLLPSWAITLISVNGIFVICCLTYCFAPRCRERRRNERLRRESVRPVGSGSGSGSGGFNVLMVHKRSHTGERPLQCEICGFTCRQKGNLLRHIKLHTGEKPFKCHLCNYACQRRDALSGGGGEGRGSLLTCGDVEENPGPRMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 36 - protein, 1077 aa, synthetic construct
MALPVTALLLPLALLLHAARPQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEITTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMLLLVTSLLLCELPHPAFLLIPEQKLISEEDLDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHWNTTKQEHFPDNLLPSWAITLISVNGIFVICCLTYCFAPRCRERRRNERLRRESVRPVGSGSGSGSGGFNVLMVHKRSHTGERPLQCEICGFTCRQKGNLLRHIKLHTGEKPFKCHLCNYACQRRDALSGGGGEGRGSLLTCGDVEENPGPRMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 37 - protein, 1082 aa, synthetic construct
MALPVTALLLPLALLLHAARPQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEITTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMLLLVTSLLLCELPHPAFLLIPEQKLISEEDLYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNEDWNTTKQEHFPDNLLPSWAITLISVNGIFVICCLTYCFAPRCRERRRNERLRRESVRPVGSGSGSGSGGFNVLMVHKRSHTGERPLQCEICGFTCRQKGNLLRHIKLHTGEKPFKCHLCNYACQRRDALSGGGGEGRGSLLTCGDVEENPGPRMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 38 - protein, 12 aa, synthetic construct
GGGSGGGSGGGS

SEQ ID NO: 39 - protein, 15 aa, synthetic construct
GGGGSGGGGSGGGGS

SEQ ID NO: 40 - protein, 20 aa, synthetic construct
GGGGSGGGGSGGGGSGGGGS

SEQ ID NO: 41 - protein, 18 aa, synthetic construct
GSTSGSGKPGSGEGSTKG

SEQ ID NO: 42 - protein, 18 aa, synthetic construct
GGSSRSSSSGGGGSGGGG

SEQ ID NO: 43 - protein, 22 aa, synthetic construct
GSGATNFSLLKQAGDVEENPGP

SEQ ID NO: 44 - protein, 24 aa, synthetic construct
SGGGGEGRGSLLTCGDVEENPGPR

SEQ ID NO: 45 - protein, 6 aa, synthetic construct
HHHHHH

SEQ ID NO: 46 - protein, 247 aa, synthetic construct
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 47 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 48 - protein, 69 aa, synthetic construct; identical to SEQ ID NO: 47

SEQ ID NO: 49 - protein, 42 aa, synthetic construct
KRGRKKLLYIFKQPFMRPVQTTQEEDGCSCRFPEEEEGGCEL

SEQ ID NO: 50 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 41 aa, synthetic construct
RSKRSRLLHSDYMNMTPRRPGPTRKHYQPYAPPRDFAAYRS

SEQ ID NO: 52 - protein, 21 aa, synthetic construct
MALPVTALLLPLALLLHAARP

SEQ ID NO: 53 - protein, 21 aa, synthetic construct
METDTLLLWVLLLWVPGSTGD

SEQ ID NO: 54 - protein, 470 aa, synthetic construct
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 491 aa, synthetic construct
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 483 aa, synthetic construct
MALPVTALLLPLALLLHAARPQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEITTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 236 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK